(19) United States
(12) Patent Application Publication
(10) Pub. No.: US 2025/0166729 A1
(43) Pub. Date: May 22, 2025

(54) ANALYSIS OF HLA ALLELES TRANSCRIPTIONAL DEREGULATION

(71) Applicants: The Francis Crick Institute Limited, London (GB); University College London, London (GB)

(72) Inventors: Clare Puttick, London (GB); Charles Swanton, London (GB); Nicholas McGranahan, London (GB)

(21) Appl. No.: 18/843,121

(22) PCT Filed: Apr. 5, 2023
(86) PCT No.: PCT/EP2023/059039

§ 371 Date: Aug. 30, 2024

(30) Foreign Application Priority Data
Apr. 7, 2022 (GB) 2205135.3
Dec. 29, 2022 (GB) 2219807.1

(51) Int. Cl. G16B 25/10; G16B 15/30; G16B 20/10; G16B 20/20; G16B 30/10; G16H 20/17; G16H 50/70

(52) U.S. Cl. CPC G16B 25/10; G16B 15/30; G16B 20/10; G16B 20/20; G16B 30/10; G16H 20/17; G16H 50/70

(57) ABSTRACT

Methods for determining whether an HLA gene has deregulated expression in a sample from a subject. The methods comprise obtaining RNA sequence data from the sample; obtaining a reference sequence that is specific to one or more HLA alleles identified to be present in the subject; and aligning the RNA sequence data from the sample to the genomic reference sequence and determining whether one or more HLA alleles in the reference sequence have deregulated expression based on one or more metrics derived from the aligned RNA sequence data at mismatch positions between homologous alleles and/or at one or more non-canonical splice junctions in the reference sequence, and one or more corresponding reference values. Methods of providing or identifying a therapy, as well as prognostic methods are also described, as well as related systems and products.

### FIELD OF THE DISCLOSURE

The present disclosure relates to methods of determining whether the expression of an HLA allele is deregulated in a sample, for example a tumour sample from a subject. Systems and related products for implementing such methods are also provided. Also described are methods of providing a therapy for a subject, the therapy targeting an antigen predicted to be presented by an HLA allele which has been determined to not have deregulated expression in a tumour of the subject, and methods of predicting response to immunotherapy.

### BACKGROUND

Emerging data has highlighted the importance of considering cancer development and evolution in the context of the immune microenvironment {Rooney, 2015; McGranahan, 2016}. Indeed, it is now clear that immune evasion represents a defining feature of cancer development, even in early stage tumours in the absence of prior systemic therapy {Hanahan & Weinberg, 2011}. Cancer immunotherapies, including immune checkpoint blockade therapy, have revolutionized cancer treatment strategies. These treatments effectively work by counteracting immune evasion and shifting the balance to immune activation, thereby facilitating T cell-mediated cancer cell elimination. However, only a subset of patients benefit from immunotherapies, emphasizing the need to identify the genomic and molecular determinants underpinning immune evasion to improve outcomes in this disease.

A key source of cytotoxic T cell response and immune activation in cancer is somatic mutations and their associated neoantigens, cancer cell specific mutations resulting in mutant peptides that elicit a T cell mediated immune response {Rooney, 2015; McGranahan, 2016}. A high tumour mutation burden (TMB) has been found to be associated with improved response to immune checkpoint blockade {Rizvi, 2015; Snyder 2014}. Importantly, however, a mutation can only engender a neoantigen and a T cell response if the associated mutant peptide is presented to T cells by human leukocyte antigen (HLA) molecules. As such, understanding the extent of HLA disruption during cancer development may have important implications for immune evasion and cancer evolution, and the development of novel treatment strategies.

Supporting the importance of HLA disruption in cancer, loss and/or down regulation of the HLA class I and II genes has been shown to occur across many cancer types {Schaafsma et al., 2021, McGranahan et al., 2017, Momburg et al., 1986}. Previous work has revealed that HLA loss of heterozygosity (LOH), whereby one of the parental alleles is lost during cancer evolution, occurs in 40% of non small cell lung cancers (NSCLCs) {McGranahan, 2017}, and recent work suggests this is one of the most pervasive mechanisms of immune evasion across cancers {Hartwig, 2022}. It has further been demonstrated that cancer subclones exhibiting HLA LOH harbour an elevated burden of non-synonymous mutations compared to their sister subclones without HLA LOH {McGranahan, 2017} and that disruption to antigen presenting machinery represents a key feature of lung cancer development {Rosenthal, 2019}. However, although the importance of HLA disruption during cancer evolution is becoming increasingly clear, the extent and importance of allele specific HLA expression in both cancer and normal tissue remains unclear.

### SUMMARY OF THE DISCLOSURE

Broadly, the present inventors recognised that although down-regulation of class I and II HLA genes, and allele specific class I HLA loss has been documented, the extent and importance of allele specific HLA expression in both cancer and normal tissue remains unclear. As the HLA locus is extremely polymorphic, the inventors previously demonstrated that a patient specific approach (taking into account germline HLA haplotypes) was advantageous to evaluate HLA allele specific copy number in cancer samples {McGranahan, 2017}. Others have built upon this work since, extending the concept to HLA expression by using a patient specific transcriptomic reference to analyse expression in tumour samples (see e.g. Aguiar et al., Filip et al.). In the present work, the inventors recognised that transcriptional deregulation investigation would benefit from the use of a patient specific genomic reference instead of a patient specific transcriptomic reference. Indeed, the inventors showed that this enables the determination of both transcriptional repression and alternative splicing, both of which are demonstrated herein to significantly contribute to HLA dysfunction in tumours. The inventors further showed that highly variable expression of HLA alleles is observed in tumour adjacent normal samples, and thus that previous approaches that assess HLA repression without reference to a comparative normal level of expression may fail to capture the relevance of any level of expression in tumour samples. In other words, contrary to what has been assumed in the prior art, the levels of HLA allele expression in tumour samples cannot provide an accurate indication of HLA repression in the tumour. Thus, the inventors developed a new method to enable analysis of allele specific HLA expression at both the class I and class II loci, taking RNA and DNA data as input (from tumour and matched normal) and predicting the fractional allele specific copy number, the expression of each HLA allele, including both class I and class II alleles within individual tumour samples, as well as alternative splicing (e.g. exon skipping) by identifying high-quality split sequencing reads. They demonstrate by using this method to analyse a TRACERx (TRAcking Cancer Evolution through therapy (Rx)) lung cancer data-set work that to fully capture the extent of HLA disruption in cancer it is imperative to consider both the DNA and RNA (Example 1). They further show that this improved assessment of HLA regulation and disruption in cancer and normal tissue and can be harnessed to help improve our ability to predict survival, study tumour evolution (Example 3) and predict response to immune checkpoint blockade therapy (Example 4). They further demonstrate that HLA alternative splicing (e.g. exon skipping) can be identified in both tumour and tumour-adjacent normal tissue, and can help further elucidate mechanisms of immune evasion in cancer (Example 2).

Accordingly, in a first aspect the present disclosure provides a method for determining whether an HLA gene has deregulated expression in a tumour sample from a subject, the method comprising: (a) obtaining RNA sequence data from the sample; (b) obtaining a reference sequence that is specific to one or more HLA alleles identified to be present in the subject; (c) aligning the RNA sequence data from the sample to the reference sequence and (d) determining whether one or more HLA alleles in the reference sequence have deregulated expression based on: (i) one or more metrics derived from the aligned RNA sequence data at one or more mismatch positions between homologous alleles and/or at one or more non-canonical splice junctions in the reference sequence, and (ii) one or more corresponding reference values.

Also described according to a second aspect is a method for determining whether an HLA gene has deregulated expression in a sample from a subject, the method comprising: obtaining RNA sequence data from the sample; obtaining a genomic reference sequence that is specific to HLA alleles identified to be present in the subject; aligning the RNA sequence data from the sample to the genomic reference sequence and determining whether one or more HLA alleles in the reference sequence have deregulated expression based on the RNA sequence data at mismatch positions between homologous alleles and/or at splice junctions in the reference sequence.

The methods of the first and second aspects may have any one or more of the following optional features.

The deregulated expression may be an altered level of expression of a specific HLA allele compared to a control level. The deregulated expression may be the presence of an alternative splicing event, such as an exon skipping event or intron retention event, in the HLA allele. An alternative splicing event may be an exon skipping event, an intron retention event, a partial exon skipping event, a complete exon skipping event, a partial intron retention event or complete intron retention event.

According to the first aspect, the deregulated expression may comprise an altered level of expression of a specific HLA allele compared to a control level defined by the one or more corresponding reference values. The one or more corresponding reference values may be expression levels in one or more normal samples. The deregulated expression may comprise the presence of an alternative splicing event in the HLA allele. The deregulated expression may be the presence of an alternative splicing event that is not present in one or more normal samples. Determining whether one or more HLA alleles in the reference sequence have deregulated expression may comprise determining the number of reads in the aligned RNA sequence data that map, optionally uniquely, to a non-canonical splice junction, wherein the one or more corresponding reference values comprise a predetermined threshold and an alternative splicing event is determined to be present in an HLA allele in the sample if the number of reads that map to a non-canonical splice junction is above a predetermined threshold. The threshold may be 20 uniquely mapping reads.

According to any aspect, the RNA sequence data may be next generation sequencing data, short reads sequence data, and/or whole transcriptome sequencing data. The RNA sequence data may be bulk RNA sequence data or single cell RNA sequencing data. The reference sequence may be a genomic reference sequence or a transcriptomic reference sequence. Obtaining the reference sequence may comprise combining reference sequences for a plurality of HLA allele previously identified to be present in the subject. Obtaining the reference sequence may comprise identifying one or more HLA alleles present in the subject and combining reference sequences for the one or more alleles identified to be present in the subject. The HLA alleles present in the subject may be identified or have been identified using DNA sequence data from the sample or from a matched sample. A matched sample may be a sample that has been obtained from the same subject. For example, when the sample may be a tumour sample from a subject, the matched sample may be a normal sample from the same subject. The HLA alleles present in the subject may be identified or may have been identified to a level of resolution that specifies at least the allele group and the specific HLA protein. The sample may be a tumour sample and the deregulated expression may be an altered level of expression of a specific HLA allele compared to a control level corresponding to one or more normal samples.

According to the first aspect, the method may comprise obtaining one or more corresponding reference values by: obtaining RNA sequence data from one or more normal samples, and for each normal sample: aligning the aligning the RNA sequence data from the normal sample to the reference sequence, and obtaining one or more metrics derived from the aligned RNA sequence data from the normal sample at one or more mismatch positions between homologous alleles and/or at one or more non-canonical splice junctions in the reference sequence. The deregulated expression may comprise an altered level of expression of a specific HLA allele, wherein one or more corresponding reference values are metrics derived from the aligned RNA sequence data from the normal sample(s) at one or more mismatch positions between homologous alleles. The deregulated expression may comprise the presence of an alternative splicing event, and one or more corresponding reference values are derived from the aligned RNA sequence data from the normal sample(s) at one or more non-canonical splice junctions in the reference sequence. The one or more metrics derived from the aligned sequence data from the tumour may comprise read depths at a plurality of mismatch positions between homologous alleles and the one or more corresponding reference values may comprise read depths at the plurality of mismatch positions between homologous alleles derived from the aligned sequence data from the one or more normal samples. Determining whether one or more HLA alleles in the reference sequence have deregulated expression may comprise comparing the read depths at mismatch positions in the tumour sample and the normal sample. The comparing may be performed using a statistical test to assess the difference between two sets of observations. The statistical test may be a paired t-test or a Wilcoxon test. The one or more metrics derived from the aligned sequence data from the tumour may comprise the number of reads that include a non-canonical splice junction and the one or more corresponding reference values may comprise the number of reads that include the non-canonical splice junction in the aligned sequence data from the one or more normal samples.

Determining whether one or more HLA alleles in the reference sequence have deregulated expression may comprise comparing the number of reads that include the non-canonical splice junction in the tumour sample and the one or more normal samples or information derived therefrom. The information derived from said number of reads may comprises an indication of whether the respective numbers of reads indicates the presence of an alternative splicing event in the tumour sample and in the normal sample(s), respectively. The number of reads that include the non-canonical splice junction in the normal or tumour sample may be considered to indicate the presence of an alternative splicing event if it is above a predetermined threshold, such as e.g. 5, 10, 15, 20 or 25 reads. For example, the number of reads that include the non-canonical splice junctions in the normal sample may be considered to indicate the presence of an alternative splicing event if it is above 20 reads. Similarly, the number of reads that include the non-canonical splice junction in the tumour sample may be considered to indicate the presence of an alternative splicing event if it is above 20 reads. Comparing the number of reads that include the non-canonical splice junction may comprise determining that the number of reads that include the non-canonical splice junction is above the predetermined threshold in the tumour sample and below the predetermined threshold in the normal sample(s). In such cases, the method may comprise determining that the HLA allele comprising the non-canonical splice junction is deregulated in the tumour. Comparing the number of reads that include the non-canonical splice junction may comprise determining that the number of reads that include the non-canonical splice junction is above the predetermined threshold in the tumour sample and above the predetermined threshold in the normal sample. In such cases, the method may comprise determining that the HLA allele comprising the non-canonical splice junction is not deregulated in the tumour or not deregulated in a tumour specific manner.

The method may comprise obtaining RNA sequence data from a matched normal sample, and aligning the aligning the RNA sequence data from the matched normal sample to the genomic reference sequence. Determining whether one or more HLA alleles in the reference sequence have deregulated expression may comprise comparing the RNA sequence data from the matched normal sample and from the tumour sample at mismatch positions between homologous alleles. Comparing the RNA sequence data from the matched normal sample and from the tumour sample at mismatch positions between homologous alleles may comprise comparing the read depths at mismatch positions in the tumour sample and the normal sample using a statistical test to assess the difference between two sets of observations. The statistical test may be a paired t test or a Wilcoxon test. For example, the distribution of reads depths at mismatch positions in the normal sample and the tumour sample may be compared using a Wilcoxon test and if the test may be associated with a p-value below a threshold e.g. 0.001, 0.005, 0.01, 0.05, or 0.1 the allele may be considered to be deregulated in the tumour compared to the normal sample. The read depths may be normalised prior to comparison, for example based on the total number of reads obtained from the sample.

The deregulated expression may be an altered level of expression of a specific HLA allele compared to a control level corresponding to a plurality of normal samples with the same HLA allele, or to a control level corresponding to a matched normal sample. Thus, the method may comprise comparing the read depths at mismatch positions in the tumour sample and a plurality of normal samples with the same HLA allele using a statistical test to assess the difference between two sets of observations. For example, a paired t test or a Wilcoxon test may be used. The read depth may be a summarised and/or normalised read depth. The control level corresponding to a plurality of normal samples with the same allele may be provided as a summarised value or as a plurality of values. Thus, according to the first aspect, the one or more reference values may be derived from one or more normal samples, where the one or more normal samples may be matched normal samples or normal samples with the same HLA allele as the tumour sample. In other words, the one or more corresponding reference values may be obtained by analysing one or more matched normal samples or by analysing a plurality of normal samples with the same allele, that may comprise exclusively matched normal samples or may not comprise any matched normal sample.

According to any aspect, the RNA sequence data may comprise RNA sequencing reads. The method may comprise obtaining a normalised read depth for one or more HLA alleles, wherein the normalised reads depth may be normalised for total coverage and/or allele length. The method may further comprise calculating an adjusted read number for one or more HLA alleles, wherein the adjusted read number for an allele takes into account the number of reads that map uniquely to the allele, the number of reads that map uniquely to the homologous allele, and the number of reads that map to both the allele and the homologous allele. A normalised read depth may be obtained by dividing the number of reads that map uniquely to an allele by the length of the allele and/or by dividing: (i) the number of reads that map uniquely to an allele, optionally normalised to the length of the allele, by (ii) the sum of the numbers in (i) for all alleles of the same gene, all alleles of genes of the same class of HLA genes, or all alleles of all HLA genes assessed. Thus, the method may comprise calculating a read depth using reads that map uniquely to an allele. Reads that do not map uniquely to an allele may be added to the unique allelic read count using the ratio of uniquely mapped reads between homologous alleles. A normalised read depth may be obtained by dividing the (optionally adjusted) number of reads that map to an allele by the length of the allele and/or by dividing: (i) the number of reads that map to an allele, optionally normalised to the total coverage in the sample, by (ii) the number of reads in the RNA sequence data for the sample. An adjusted read depth may be obtained by adding (i) the number of reads that map uniquely to the allele, and (ii) the number of reads that map to both the allele and the homologous allele multiplied by a correction factor, wherein the correction factor is the ratio of the number of reads that map uniquely to the allele and the number of reads that map uniquely to either the allele or the homologous allele. Normalising by the total coverage in the sample may comprise dividing by the total number of reads in the RNA sequence data from the sample. Normalising by the total number of reads in the sample may enable a more accurate comparison of read depths between samples that may have been sequenced at different depths (such as e.g. a tumour sample and one or more normal samples). For example, an adjusted read number for a first allele of a pair of homologous alleles may be obtained as: R₁=r₁+f₁×r₁₂ where r1 is the number of reads that map uniquely to the first allele, r12 is the number of reads that map to both the allele and the homologous allele, and f1 is a correction factor calculated as f₁=r₁/r₁+r₂. Similarly, an adjusted read number for the second allele of the pair of homologous alleles may be obtained as: R₂=r₂+(1−f₁)×r₁₂, where r2 is the number of reads that map uniquely to the second allele. A normalised read depth may be obtained as:

$${RPKM}_{i} = \frac{{R}_{i} / l}{{a}_{i}}$$

where i is the allele (1 or 2), r1 is a read count or adjusted read count for the allele, I is the total coverage of the sample (total number of reads in the sequence data for the sample) and ai is the length of the allele.

The deregulated expression may be an altered level of expression of a specific HLA allele compared to a control level. The method may comprise excluding any allele for which the numbers of reads that map uniquely to one of the two homologous alleles may be below a predetermined threshold. The predetermined threshold may be 30%, 40%, 50% or 60%. The percentage of reads may refer to the total number of reads that map to an allele. Thus, an allele may not be determined to be specifically deregulated if, from the total number of reads mapping to said allele, less that 30%, 40%, 50% or 60% map to mismatch positions between homologous alleles (i.e. are unique to said allele). The deregulated expression may be an altered level of expression of a specific HLA gene compared to a control level. The method may comprise determining a gene level read depth based on the number of reads that map to one or both homologous alleles. The gene level read depth may be normalised for total coverage and/or allele length, such as average allele length, of the homologous alleles. In embodiments, a specific HLA gene deregulated expression may be determined for an HLA allele. This is common to a pair of homologous alleles. This may be particularly useful in cases where the numbers of reads that map uniquely to one of the two homologous alleles is below a predetermined threshold. In such embodiments, a gene level read depth may be determined instead of (or in addition to) an allele-specific read depth. A gene level read depth may be obtained using the number of reads that map uniquely to an allele of the gene (r₁), the number of reads that map uniquely to the homologous allele of the gene (r₂), and the number of reads that map to both the allele and the homologous allele of the gene (r₁₂). The gene level read depth may be normalised based on the total coverage in the sample (1) and/or the length of each of the alleles of the gene (a₁, a₂). For example, a gene level read depth may be calculated as

$$RPKM = \frac{( {r}_{1} + {r}_{2} + {r}_{12} ) / l}{( {a}_{1} + {a}_{2} ) / 2} .$$

The method may comprise determining the allelic imbalance between two homologous alleles in the sample as the ratio of the, optionally normalised, read depths at mismatch positions between the two alleles. Thus, the method may comprise determining whether there is allelic imbalance between two homologous alleles in the sample by comparing the read depths at mismatch positions between the two alleles. The comparing may use a statistical test, optionally a Wilcoxon test. The read depths may be adjusted such that each sequence read in the RNA sequence data that maps to a mismatch position is counted only once.

The HLA allele may be a class I HLA allele. The HLA allele may be a class II HLA allele.

The method may comprise excluding any HLA gene for which the numbers of, optionally selected, reads that map to more than one HLA gene is above a predetermined threshold. The predetermined threshold may be 5%, 10%, 15% or 20%.

The HLA gene may be a class I gene, and the non-canonical splice junction may involve one or more of exons 2, 3, 4, 5 and introns 2, 3, 4, 5. The non-canonical splice junction may result in partial or complete exon skipping of exon 5, partial or complete intron retention of intron 5, partial or complete exon skipping of exons 2, 3 and/or 4, partial intron retention of introns 3 and/or 4. The non-canonical splice junction may involve exon skipping of exon 3 and/or exon 5, intron retention of intron 5, complete exon skipping of exon 5, complete exon skipping of exon 3, partial intron retention of intron 5, partial skipping of exons 2, 3 and/or 4, and/or partial intron retention of introns 3 and/or 4. Determining whether one or more HLA alleles in the reference sequence have deregulated expression may comprise determining the number of reads that support the presence of an exon skipping event. An HLA allele may be determined to be deregulated in the sample if the number of reads that support the presence of an exon skipping event is above a predetermined threshold. For example, the predetermined threshold may be 10, 15, 20, 25 or 30 reads. A read that supports the presence of an exon skipping event may be a read that comprises a sequence that matches a target sequence in a set of sequences comprising all sequences of a predetermined length in the genomic reference. A read that supports the presence of an exon skipping event may be a read that comprises sequence from two exons that surround a candidate exon and do not comprise sequence from the candidate exon. The HLA gene may be a class I gene, and the exon skipping event or candidate exon may be selected from exons 3, 5 and 6. The exon skipping event or candidate exon may be exon 3 and/or exon 5. The sample may be a tumour sample. The HLA gene may be HLA-A or HLA-B, and the exon may be exon 5. The HLA gene may be HLA-C. The HLA gene may be a class II gene. The exon skipping event or candidate exon may be selected from exons 2, 3, 4 or 5 of the beta chain of a class II gene. The exon skipping event or candidate exon may be selected from exons 2, 3, or 4 of the alpha chain of a class II gene.

Obtaining the reference sequence may comprise identifying one or more non-canonical splice junctions in the reference sequence by aligning the RNA sequence data from the sample to reference sequences for one or more HLA alleles identified to be present in the subject. The method may further comprise determining the fraction of cancer cells in the sample that comprise an alternative splicing event in an HLA allele by determining the number of reads that include a non-canonical splice junction in the sample, the number of reads that include a corresponding canonical splice junction, obtaining the ratio of said numbers and dividing the ratio by an estimated cancer cell fraction for the sample. The number of reads may be reads that uniquely map to a region containing the non-canonical splice junction or to a region containing the corresponding canonical splice junction. A corresponding canonical splice junction may be a splice junction that does not contain an exon skipping event or intron retention event, and that is at the junction between: (i) the two exons involved in the non-canonical splice junction, (ii) an exon involved in the non-canonical splice junction and a skipped exon, or (iii) an exon involved in the non-canonical splice junction and a subsequent or preceding exon separated from said exon by a retained intron. The number of reads that include a corresponding canonical splice junction may be obtained as a summarised value (e.g. average) derived from the number of reads that are at the junction of each of two exons that are involved in the non-canonical splice junction and a skipped exon or plurality of skipped exons that are between the two exons involved in the non-canonical splice junction. The fraction of cancer cells in the sample that comprise an alternative splicing event in an HLA allele may be influenced by the (typically unknown) allele expression in the normal cells and tumour cells in the sample. Nevertheless, the value may represent a useful estimate of the fraction of cancer cells in the sample that comprise an alternative splicing event in an HLA allele.

A read that includes a non-canonical splice junction may be a read that comprises sequence from two exons that surround a candidate exon and do not comprise sequence from the candidate exon, a read that comprises sequence from a first exon and a subsequent exon wherein the junction between the first and subsequent exon is within the sequence of the first exon, a read that comprises sequence from a first exon and a preceding exon wherein the junction between the first and preceding exon is within the sequence of the first exon, a read that comprises sequence from an exon and at least a part of the subsequent intron, or a read that comprises sequence from an exon and at least a part of the preceding intron. A read that includes a non-canonical splice junction may be a read that comprises sequence from two exons that surround a candidate exon and do not comprise sequence from the candidate exon (skipped exon), a read that comprises sequence from a first exon and a subsequent exon wherein the junction between the first and subsequent exon is within the sequence of the first exon (partial skipping of the first exon), a read that comprises sequence from a first exon and a preceding exon wherein the junction between the first and preceding exon is within the sequence of the first exon (partial skipping of the first exon), a read that comprises sequence from an exon and at least a part of the subsequent intron (retained intron), or a read that comprises sequence from an exon and at least a part of the preceding intron (retained intron).

The sequence data may comprise sequencing reads. Aligning RNA sequence data to the reference may comprise selecting reads that align to a region comprising the HLA locus in a standard genome or transcriptome reference sequence. Aligning RNA sequence data to the reference sequence may comprise selecting reads that contain a sequence that matches to a sequence from a set of target sequences. Aligning RNA sequence data to the reference sequence may comprise selecting reads that do not align to any regions of a standard reference sequence. The match may be an exact match. The set of target sequences may be a set of k-mers created from the subject-specific reference sequence. The set of target sequences may comprise all sequences of a predetermined length, such as e.g. 30 bases, in the genomic sequences of a set of possible alleles in the subject. The region comprising the HLA locus may be selected as a chromosome or portion of chromosome. For example, chromosome 6 may be used for human samples. The set of possible alleles in the subject may comprise the alleles determined to be present in the subject. The set of possible alleles in the subject may comprise all possible alleles in the class(es) of HLA genes under investigation. The set of possible alleles in the subject may comprise all possible HLA gene alleles in the species of the subject. For example, the set of possible alleles in the subject may comprise all human alleles available in the IPD-IMGT/HLA database. For the identification of deregulated expression, aligning RNA sequence data to the reference sequence may comprise selecting reads that align to a region comprising the HLA locus in a standard reference sequence and selecting reads that do not align to any regions of a standard reference sequence. Aligning RNA sequence data to the reference sequence may further comprise selecting reads that contain a sequence that matches to a sequence from a set of target reference sequences. The target set of reference sequences may be selected as k-mers generated from the set of possible alleles in the subject. For the identification of alternative splicing events, aligning RNA sequence data to the reference sequence may comprise selecting reads that contain a sequence that matches to a sequence from a set of target reference sequences. The target set of reference sequences may be selected as k-mers generated from the set of possible alleles in the subject. This may result in a tightly constrained set of reads that provide high quality evidence for exon skipping events.

The reference sequence may be a genomic reference sequence. Aligning the RNA sequence data from the sample to the genomic reference sequence may comprise aligning the RNA sequence data or selected reads from the RNA sequence data to the genomic reference provided as a genomic sequence and an indication of the locations of introns and exons in the genomic reference sequence. The locations of introns and exons in the genomic sequence may comprise the known (canonical) locations of introns and exons. The method may comprise identifying using the aligned RNA sequence data the location of one or more candidate non-canonical splice junctions. The method may further comprise excluding candidate non-canonical splice junctions that are supported by fewer than a predetermined number of reads (such as e.g. 2, 3 or 5 reads). Aligning the RNA sequence data from the sample to the genomic reference sequence may comprise re-aligning the RNA sequence data or selected reads from the RNA sequence data to the genomic reference provided as a genomic sequence and an updated indication of the locations of introns and exons in the genomic sequence. The updated indication may comprise the known locations of introns and exons and the one or more candidate non-canonical splice junctions. The locations of introns and exons in the genomic sequence may be in the form of an indication of the location of one or more splice junctions. Conversely, the locations of one or more splice junctions in the genomic sequence may be in the form of an indication of the locations of introns and exons in the sequence (and optionally untranslated regions).

A genomic sequence can be provided as a fasta file. An indication of the locations of introns and exons in a genomic sequence can be provided as a GTF file. An updated indication of the locations of introns and exons in the genomic sequence may be obtained as the result of a first step of aligning the RNA sequence data from the sample to the genomic reference sequence for one or more samples. For example, a first step of alignment may be performed for a plurality of samples such as a cohort of patients with a similar disease as the subject. The first alignment may be performed using an indication of the locations of known introns and exons, for example locations from a sequence database such as IMGT. The location of one or more additional introns/exons and/or corresponding splice junctions may be obtained as a result of the first alignment step. One or more additional introns/exons and/or corresponding splice junctions may be identified in one or more of the plurality of samples and/or in one or more of the plurality of alleles identified for one or more of the plurality of samples. All of the additional introns/exons and/or corresponding splice junctions may be combined and included in an updated indication of the locations of introns and exons in the genomic sequence. The locations of introns and exons in the genomic sequence may comprise the locations of introns and exons that are supported by a predetermined number of reads in a sample or plurality of samples. For example, new splice junctions may only be included in an indication of the location of introns and exons if they are supported by at least e.g. 20 reads.

An alignment step may be performed using the same sequence data and/or the same reference sequence for one or more samples that are obtained from a single tumour region (such as e.g. from a tumour biopsy).

The method may comprise identifying one or more somatic mutations in the sequence of one or more HLA alleles present in the subject. Identifying one or more somatic mutations in the sequence of one or more HLA alleles present in the subject may comprise aligning DNA sequence from the sample to the genomic reference. Determining whether one or more HLA alleles in the reference sequence have deregulated expression may comprise determining the number of reads that comprise the one or more somatic mutations. The presence or absence of reads comprising somatic mutations identified in the sample provides further evidence as to whether the HLA allele may be deregulated. Indeed, the absence of reads comprising a somatic mutation in the sample indicates that the HLA allele may be likely to be repressed in the sample. The method may comprise obtaining said sample from said subject. The method may comprise obtaining RNA and/or DNA sequence data from a sample that has been previously obtained from the subject, such as e.g. by sequencing. Alternatively, the sequence data may be obtained from a user, computing device or data store.

The method may comprise providing to a user, optionally through a user interface, one or more of: one or more values quantifying the expression of one or more respective HLA alleles identified in the subject, such as a normalised read depth, one or more values quantifying the relative expression of a pair of homologous alleles identified in the subject, an indication of whether an HLA allele is determined to be deregulated in the sample, an indication of whether an HLA allele is determined to be transcriptionally repressed in the sample, an indication of whether an HLA allele is determined to have an exon skipping event in the sample, a number of reads supporting the presence of an exon skipping event in the sample, an indication of whether an HLA allele is determined to be transcriptionally repressed in a tumour sample compared to a matched normal sample, a statistical metric (e.g. p value) associated with an indication of whether an HLA allele is differentially expressed in a tumour sample compared to a control level of expression, or a value derived therefrom. The method may comprise providing to a user one or more of: one or more of the metrics derived from the aligned RNA sequence data at one or more mismatch positions between homologous alleles, or a metric derived therefrom, such as a read depth for one or both alleles of a pair of homologous alleles in the sample and/or one or more control samples, a normalised read depth for one or both alleles of a pair of homologous alleles in the sample and/or one or more control samples, one or more values quantifying the relative expression of a pair of homologous alleles identified in the subject, an indication of whether an HLA allele is determined to be deregulated in the sample, an indication of whether expression of an HLA allele is determined to be repressed in the sample compared to a control, one or more of the metrics derived from the aligned RNA sequence data at one or more non-canonical splice junctions or a metric derived therefrom, one or more metrics derived from aligned RNA sequence data from one or more normal samples at one or more non-canonical splice junctions or a metric derived therefrom, an indication of whether an HLA allele is determined to have an alternative splicing event in the sample, a number of reads that include a non-canonical splice junction in the sample, an indication of whether an HLA allele is determined to have an alternative splicing event in one or more normal samples, a number of reads that include a non-canonical splice junction in one or more normal samples, a statistical metric (e.g. p value) associated with an indication of whether an HLA allele is differentially expressed in a tumour sample compared to a control level of expression, or a value derived therefrom. A value derived from any of the above may comprise a diagnostic or prognostic indication.

As the skilled person understands, the complexity of the operations described herein (due at least to the amount of data that may be typically generated by RNA sequencing) are such that they are beyond the reach of a mental activity. Thus, unless context indicates otherwise (e.g. where sample preparation or acquisition steps are described), all steps of the methods described herein are computer implemented.

According to a further aspect, there is provided a method of providing an immunotherapy for a subject, the method comprising: (i) identifying one or more neoantigens that are present in the subject; (ii) determining whether the one or more neoantigens are predicted to be presented by an HLA molecule encoded by an HLA allele that may be deregulated in the subject using the method of any embodiment of the first or second aspect; and (iii) providing an immunotherapy that targets a neoantigen of the one or more neoantigens that may be predicted to be presented by an HLA molecule encoded by an HLA allele that may be not deregulated in the subject.

According to a further aspect, there is provided a method of identifying a therapy for a subject that has been diagnosed as having cancer, the method comprising: (i) identifying one or more neoantigens in the subject to obtain a first neoantigen burden for the subject; (ii) carrying out the method of the first or second aspect on one or more samples (e.g. tumour samples) from the subject to determine whether one or more HLA alleles are deregulated in the subject; (iii) adjusting the first neoantigen burden for the subject to exclude neoantigens predicted to bind to an HLA allele that has been determined at step (ii) to be deregulated in a tumour from the subject; and (iv) classifying the subject between a plurality of groups associated with a different responses to CPI therapy based on the adjusted neoantigen burden obtained at step (iii). Step (iii) may comprise excluding any neoantigen that may be predicted to bind only to alleles that are determined to be deregulated in a tumour from the subject. Step (iii) may comprise excluding any neoantigen that may be determined to be lost and/or repressed and/or with skipping of exon 3 in a tumour from the subject. Step (i) may comprise identifying a plurality of somatic mutations present in the subject. Step (i) may further comprise identifying neoantigens as mutations of the plurality of somatic mutations that lead to a protein or peptide that may be not expressed in normal cells. Step (i) may further comprise determining a first tumour mutational burden as the number of somatic mutations present in the subject. Step (iii) may comprise adjusting the first TMB for the subject by excluding any mutations that leads to a neoantigen that may be predicted to bind to an HLA allele that has been determined at step (ii) to be deregulated in a tumour from the subject. Thus, the present aspects also relates to a method of identifying a therapy for a subject that has been diagnosed as having cancer, the method comprising determining a first TMB for the subject and adjusting the first TMB to exclude mutations that lead to neoantigens predicted to bind to an HLA allele that has been determined at step (ii) to be deregulated in a tumour from the subject. The plurality of groups may comprise a first group associated with a higher adjusted TMB than a second group, wherein the first group has a better response to CPI therapy than the second group. The method may further comprise selecting the subject for treatment with CPI therapy if the subject may be classified in the first group. The method may further comprise selecting the subject for treatment with an alternative therapy if the subject may be not classified in the first group.

Also described herein is a method of providing a prognosis for a subject, the method comprising: (i) determining whether an HLA gene has deregulated expression in a tumour sample from said subject using the method of any embodiment of the first aspect, and (ii) classifying the subject between at least first category and a second category based on the one or more metrics obtained at step (ii) or information derived therefrom, such as e.g. a metric quantifying the total expression of all HLA alleles identified to be present in the subject (e.g. obtained as a sum or average of the read depths associated with each of the HLA alleles identified to be present in the subject, optionally summarised across a plurality of samples from the subject, such as by taking the minimum or average across the samples), wherein the first category has a higher overall survival and/or a lower likelihood of metastasis than the second category.

The method of any preceding aspect may further comprise determining whether one or more HLA alleles are lost in the subject, for example using a method as described herein or in WO 2019/012296. Providing an immunotherapy may comprise providing an immunotherapy that targets a neoantigen of the one or more neoantigens that is predicted to be presented by an HLA molecule encoded by an HLA allele that is not deregulated in the subject and that is not lost in the subject. Adjusting the first neoantigen burden for the subject may further comprise excluding neoantigens predicted to bind to an HLA allele that has been determined to be lost in a tumour from the subject.

According to a further aspect, the present disclosure provides a system comprising: at least one processor; and at least one non-transitory computer readable medium containing instructions that, when executed by the at least one processor, cause the at least one processor to perform the steps of any embodiment of any preceding aspect.

The system according to the present aspect may be configured to implement the method of any embodiment of the preceding aspects. In particular, the at least one non-transitory computer readable medium may contain instructions that, when executed by the at least one processor, cause the at least one processor to perform operations comprising any of the operations described in relation to the first or second aspects. The system may further comprise, in operable connection with the processor, one or more of: a user interface, wherein the instructions further cause the processor to provide, to the user interface for outputting to a user, one or more of: an expression value (e.g. transcripts per thousand) for one or more HLA alleles, an indication of whether an HLA allele is differentially expressed in a tumour sample compared to a normal level of expression, a statistical metric (e.g. p value) associated with an indication of whether an HLA allele is differentially expressed in a tumour sample compared to a normal level of expression, an indication of whether an HLA allele is subject to exon skipping, a number of reads supporting the presence of an exon skipping event in an HLA allele, or a value derived therefrom; one or more sequence data acquisition device (such as e.g. a next generation sequencer); one or more data stores, such as e.g. a sequence data store, reference sequence data store, etc.

According to a further aspect, there is provided a non-transitory computer readable medium or media comprising instructions that, when executed by at least one processor, cause the at least one processor to perform the method of any embodiment of any aspect described herein.

According to a further aspect, there is provided a computer program comprising code which, when the code is executed on a computer, causes the computer to perform the method of any embodiment of any aspect described herein.

The present invention includes the combination of the aspects and preferred features described except where such a combination is clearly impermissible or is stated to be expressly avoided. These and further aspects and embodiments of the invention are described in further detail below and with reference to the accompanying examples and figures.

### BRIEF DESCRIPTION OF THE FIGURES

FIG. 1 is a flowchart illustrating schematically a method of determining whether the expression of an HLA allele is deregulated in a sample.

FIG. 2 shows an embodiment of a system for determining whether the expression of an HLA allele is deregulated in a sample.

FIG. 3 illustrates schematically: A. a method of determining whether the expression of an HLA allele is deregulate in a sample, comprising mapping reads to a subject-specific genomic reference and identifying reads aligning to mismatch positions between two HLA alleles; and B. A tool for determining HLA DNA and RNA disruption.

FIG. 4 shows the results of an analysis of HLA allelic expression in normal tissue. A. The total gene level expression in the tumour adjacent normal samples for HLA-A, HLA-B, and HLA-C. C. The HLA allele expression grouper allele type in the tumour adjacent normal samples. B. The fraction of tumour adjacent normal samples that have allelic imbalance (AIB) (upper panel), the allelic imbalance (AIB) ratio (lower panel).

FIG. 5 shows the results of an analysis of HLA expression tumour regions and tumour adjacent normal samples. A. HLA class I alleles. B. HLA class II alleles. C. Fraction of tumour and tumour adjacent normal samples that have allelic imbalance (AIB) (upper panel) and AIB ratio (lower panel) in tumour and normal samples.

FIG. 6 shows schematically an analysis of HLA repression. HLA repression in the tumour regions is called relative to the matched tumour adjacent normal samples. An HLA allele is defined as repressed if there is a significant difference in the normalised read counts at the mismatch positions, using a paired Wilcoxon test. In this example allele 2 is repressed in the tumour region compared to the tumour adjacent normal sample.

FIG. 7 shows the HLA LOH and repression in the TRACERx cohort. A Each column represents a patient/tumour, each box represents a region from that tumour. Above the x axis the boxes are coloured by the HLA LOH status of the region (blue=LOH) Below the x-axis the boxes are coloured by whether the region has transcriptional repression of the same allele that is lost in the DNA (blue, if there is LOH in the DNA), the alternate allele (red) or both alleles (purple). B. The number of patients with HLA LOH and/or repression. The data shows that without an exploration of RNA expression, the extent of HLA disruption is significantly underestimated. C. The ratio of allelic expression in the tumour region compared to the patient-matched normal sample when the allele has LOH or repression. D. The frequency of monoallelic and biallelic repression events. E. In genes where there is biallelic repression, the ratio of tumour to normal allelic expression is shown. F. The total number of intact alleles when accounting for alleles disrupted by LOH and repression. G. For each tumour region, the fraction of putative neoantigens predicted to bind exclusively to HLA alleles subject to LOH or repression. H. Effective neoantigen count that takes into account if a neoantigen is no longer presented to T cells due to HLA dysregulation, in the TRACERx cohort.

FIG. 8 shows number of intact HLA class I alleles, taking into account HLA LOH and repression, in the CPI1000+ cohort. Using the effective neoantigen metric (or a corresponding tumour mutational burden, TMB metric) it is possible to better predict which patients will respond to immunotherapy in the CPI1000+ cohort.

FIG. 9 shows how many tumour regions have zero one or two of their alleles downregulated, in the TRACERx data analysed.

FIG. 10 shows the variation in HLA allelic expression in tumour adjacent normal tissue. A For each gene the expression of the higher and lower allele. B. The gene level expression. C. The fraction of tumour adjacent normal samples that have allelic imbalance (AIB) (upper panel), the AIB ratio (lower panel), for class II alleles. E. HLA allele expression by allele group, for the class II alleles. F. Relationship between the allele repertoire size and allele expression. As the repertoire size increases, i.e. the allele can bind more peptides, the expression decreases.

FIG. 11 shows that in tumour regions that have LOH, there is a negative correlation between the purity (fraction of cells that are cancer cells) and the expression of the lost allele, as would be expected. As the allele is lost in the DNA in the cancer regions, all the expression that measured must be coming from the non cancer cells, the number of which increases as purity decreases. Each plot shows this relationship for the respective HLA-A, -B and -C genes (with a point for each sample), and the right bottom plot shows the data for the A-C genes (top and bottom left plots) together on the same plot.

FIG. 12 shows an analysis of the number of clonal neoantigens predicted to bind to each allele in tumour samples. A. Difference in the number of clonal neoantigens that are predicted to bind to each allele. B. Ratio of the maximum number of neoantigens to the minimum number of neoantigens. C. Allele expression for the alleles in A. This plot shows that the number of neoantigens predicted to bind to each allele in a pair differs slightly. At least for alleles that are lost (where LOH is identified), deregulated alleles tend to be associated with higher number of predicted neoantigens.

FIG. 13 shows an analysis of the number of clonal and subclonal neoantigens predicted to bind to haplotypes and alleles in tumour samples. A. Number of clonal neoantigens predicted to bind to a haplotype. Haplotypes were defined using the results of an analysis of LOH from WES data as described in MCGranahan et al., 2017. If there was AIB in the DNA, the major allele was defined as the one from the allele with the higher copy number. The upper plot shows the difference in the number of clonal neoantigens that are predicted to bind to each allele, the—middle plot shows the ratio of the maximum number of neoantigens to the minimum number of neoantigens, and the lower plot shows the expression of the haplotypes. B. Shows the same as A but for subclonal neoantigens. C. Same as A but for clonal neoantigens considered to be expressed. The upper plot shows the difference in the number of expressed clonal neoantigens that are predicted to bind to each allele. The middle plot shows the ratio of the maximum number of expressed neoantigens to the minimum number of expressed neoantigens. The bottom lot shows the allele expression. A neoantigen is defined as expressed if it is found in at least 4 reads in the RNA. D. Same as C but for subclonal neoantigens.

FIG. 14 shows tumour samples data clustered based on the HLA allele expression (‘expression group’) and HLA allele expression ratio (‘expression ratio group2, expression of the highest expressed allele to the lowest for each gene). A1 is the expression of the HLA-A allele with the highest expression for that sample, A2 is the expression of the HLA-A allele with the lowest expression. Same for B1, B2, C1 and C2. The plot also shows the total HLA expression, and the ratio of expression of alleles A1/A2, B1/B2 and C1/C2, the expression of key marker genes, the immune group (Rosenthal et al., 2019), the TCRA score and the Danaher scores for CD8 and NK cells (Danaher et al., 2018), the CD4 T cell score from the Davoli method (Davoli et al., 2017), the sample type, cancer stage, smoking status, and the purity of the samples. The bottom plot shoes a dot for each tumour region in a patient, with lines connecting the multiple regions in a patient. This shows that different tumour regions from the same patient can have very variable expression. Looking at the Immune group, the TCRA score from Bentham et al. (2021) and the Danaher scores for immune cells, it looks like regions with higher HLA allele expression are more likely to have higher HLA expression. A for LUAD B for LUSC. The legend on panel B applies to both A and B.

FIG. 15 shows the relationship between the total HLA gene expression (A, B and C together) and the CD8 T cell score from the Danaher method (A), the NK score from the Danaher method (B) the CD4 T cell score from the Davoli method (C) and The TCRA score. This shows that the HLA expression estimates obtained using the methods described herein are consistent with expected patterns for immune metrics.

FIG. 16 shows a comparison between results for HLA expression obtained using methods described herein and using RSEM.

FIG. 17 illustrates schematically the rationale behind analysis of HLA exon skipping. A. A cartoon outlining the structure of the HLA molecule. The protein domains are coloured by the HLA exons that encode them, as shown in FIG. 18B. B. Schematic representation of an HLA receptor protein sequence with exons and protein domains (P=peptide binding grove, formed at interface between α1 and α2, TM=transmembrane region, CYT=cytoplasmic tail, S=signal peptide). Exon skipping of exon 3 could lead to less HLA alleles on the cell surface, or decoy HLA alleles on the cell surface. Exon skipping of exon 5 could lead to soluble HLA molecules. Soluble HLA molecules from the cancer cells could cause immune inhibition. For example, they could engage with T cells and NK cells in the tumour microenvironment.

FIG. 18 shows results of an analysis of HLA exon skipping in the TRACERx cohort. A. The data shows exon skipping in HLA exons 3 and 5 in tumour regions and tumour adjacent normal samples. For HLA-A/B/C skipped exons identified by mapping split reads are shown. Each point represents a tumour sample (blue) or a normal sample (red). Only samples with any exon skipping are shown. Notably, for HLA-A and HLA-B exon skipping is almost exclusively observed in tumour samples. B. The fraction of LUAD and LUSC tumours with cancer cell specific HLA alternative splicing events. The exons are coloured by the protein domains they encode as shown in FIG. 17A. C. The number of somatic alternatively spliced events observed across the tumours with tumour-adjacent normal samples. D. The consequences of the alternative splicing events. E. The purity scaled novel transcript ratio for the somatic alternative spliced events. F. The fraction of tumours that have alternatively spliced events across 130 oncogenes and tumour suppressor genes. G. The fraction of tumour regions that do/do not have repression and do/do not have somatic alternatively spliced events. H. The number of neoantigens predicted to bind to either the alternatively spliced allele or non-alternatively spliced allele. The alleles are split by whether there is no somatic alternative splicing or somatic alternative splicing of exons or introns 2, 3 or 4.

FIG. 19 shows the results of an analysis of exon skipping in class I HLA alleles in the TRACERx cohort. A-C. Exon skipping events by HLA allele type Exon 5 skipping in HLA-C occurs more frequently in certain allele types, contrasted with exon 5 skipping in HLA-A and exon 3 skipping in HLA-B. D. The number of exon skipping events split by whether they were detected in alleles that have been lost in the DNA. Notably, only skipping of exon 5 in HLA-C is detected in lost alleles, suggesting that these events are occurring in the non-cancer cells of the tumour.

FIG. 20 shows the results of an analysis of LOH in class I HLA alleles in tumour samples in the TRACERx cohort. A. Loss of heterozygosity rates in the primary tumours of the TRACERx421 cohort. The 6 different NSCLC histological subtypes are shown on the x-axis. B. Proportion of LOH that was clonal or subclonal, by sample type.

FIG. 21 shows an analysis of the role of HLA disruption in tumour evolution, in the TRACERx cohort. A The heterogeneity of the HLA LOH, repression and somatic alternatively spliced events. B, C Overview (B) and examples (C) of parallel evolution, where the same allele is disrupted via different mechanisms in different regions of the same tumour. D The relationship between the presence of LOH or repression in a tumour region and the amount of CD8 T cell infiltration. E The relationship between the presence of somatic alternative splicing in a tumour region and the amount of CD8 T cell infiltration. F Tumour regions with and without HLA loss of heterozygosity have similar levels of HLA expression. G Survival curves where patients are split by the minimum total HLA expression across all regions. H LUAD tumours that have HLA LOH are more likely to metastasise.

FIG. 22 illustrates a process for filtering full exon skipping partial exon skipping and partial intron retention events. Each novel splice junction with less than 20 uniquely mapping supporting reads is filtered and removed from further analysis.

FIG. 23 is a consort diagram illustrating the number of samples used for some of the analyses described herein.

FIG. 24 shows results of an analysis confirming HLA repression calls made using a method as described herein with HLA LOH calls made using a method as described herein. A The fraction of tumour regions predicted to have HLA LOH using the WES data, coloured by the HLA repression status. B The purity of the tumour regions that are predicted to have HLA LOH from the WES data, coloured by the HLA repression status.

FIG. 25 illustrates schematically types of alternative splicing that may be detected according to embodiments of the methods described herein.

FIG. 26 shows results of an embodiment of a process of defining cancer cell specific alternative splicing events as described herein. A The number of alternatively spliced events occurring in alleles with and without genomic loss. B The heterogeneity of non-cancer cell specific alternatively spliced events.

FIG. 27 shows results of an embodiment of a method as described for detecting full intro retention.

FIG. 28 shows that alleles with alternatively spliced events introducing a premature termination codon (PTC) are not enriched for repression.

FIG. 29 shows results of analyses of non-cancer cell specific alternatively spliced events using methods as described herein. A The number of non-cancer cell specific events per tumour with a tumour-adjacent normal sample. B, C, D The number of events by allele type, for HLA-A, (B), HLA-B (C) and HLA-C (D). E The consequence of the non-cancer cell specific alternatively spliced events. F The novel transcript ratio of the non-cancer cell specific alternatively spliced events.

FIG. 30 shows results of an analysis of tumour region CD8 T cell infiltrate levels. A,B There is a positive correlation between total HLA expression and CD8 T cells, regardless of the HLA LOH status of the tumour region.

### DETAILED DESCRIPTION

The present inventors developed the first patient specific approach to evaluate HLA allele specific copy number in cancer samples, called LOHHLA (Loss of Heterogyzosity in Human Leukocyte Antigen), {McGranahan, 2017}. This is described in WO2019/012296. The approach comprises aligning HLA genomic sequence reads from a tumour sample from a subject with an HLA allele reference sequence which is based on the subject's HLA type, determining mismatch positions in homologous HLA alleles, determining mismatch coverage for each HLA allele, determining the log R (tumour/normal coverage ratio) from the mismatch coverage, determining the B allele fraction (BAF) at each polymorphic site, and determining the major and minor allele copy number at each polymorphic site using the log R value from the region in which the polymorphic site was found and the BAF of the polymorphic site (also taking into account estimated tumour purity and ploidy obtained using e.g. ASCAT {van Loo et al., 2010}). This approach enabled the first wide scale evaluation of HLA loss of heterozygosity (LOH) in cancer, whereby one of the parental alleles is lost during cancer evolution, revealing that this occurs in 40% of non-small cell lung cancers (NSCLCs) {McGranahan, 2017}. Since then, the approach has been extended by others to investigate HLA deregulation at the transcriptomic level, using patient specific transcriptomic references. For example, Aguiar et al. {2019} described a method called HLApers to measure HLA expression from whole-transcriptome RNA-seq data. The method comprises aligning reads to reference sequences comprising the coding regions of all known HLA alleles, identifying those reference sequences which maximise the read counts at each locus to infer the genotype which is present, and using this inferred HLA genotype to create a personalised index which is used to quantify expression. Thus, this approach uses the same RNA sequence data from a single sample for both the HLA typing and the expression quantification step, using a 2-step alignment process for that sequence data. Thus, only transcriptomic data and transcriptomic reference sequences are used, together with RNAseq data from a single sample. By contrast, the present inventors have identified that the use of a genomic reference sequence (instead or in addition to a transcriptomic reference sequence) advantageously enables to map both RNA and DNA sequencing reads (for example by providing intron/exon information together with a genomic reference sequence as an input to the alignment software used), and enables the quantification of more complex deregulation of expression than is possible using a transcriptome reference sequence alone. The present inventors have further identified that the use of a comparative normal level of expression for the quantification of allele specific expression (and optionally also allele-specific splicing) in a tumour sample enabled a much more accurate assessment of HLA repression in tumour samples due to large variability in expression levels between alleles in normal tissues. Thus, approaches that do not make use of a normal sample or corresponding level of expression (of normal but also sometimes alternatively spliced transcripts) to obtain an allele specific reference level of expression are likely to provide misleading information about HLA repression in tumours. Further, approaches that do not map RNA sequence data to a genomic reference sequence may fail to capture deregulation events that profoundly impact the function of HLA alleles. More recently, Filip et al. {2020} also attempted to quantify HLA-1 allele specific expression in tumours. They proposed a method called “arcasHLA-quant” which uses WES data from a normal sample to perform HLA class I genotyping using Polysolver {Shukla et al., 2015}. This is then used to create a customised transcriptome reference by replacing default HLA transcripts from the human chromosome 6 reference with patient specific HLA-1 allelic cDNA references obtained from the IMGT/HLA database. RNAseq data from a tumour sample is mapped to this reference and used to obtain an allele-specific expression quantification. This is corrected for tumour purity and ploidy inferred from paired tumor and normal samples. Thus, no reference level of expression in a normal sample is taken into account, and instead the DNA copy numbers and purity inferred from DNA sequence data from matched normal and tumour samples are used to correct the RNA expression estimates in the tumour sample. The present inventors have identified that this approach may lead to misleading results as it fails to capture high variability in normal levels of expression between alleles, which cannot be accounted for by correcting for purity and ploidy as these fail to capture these allele-specific differences that occur at the level of expression rather than at the genomic level. Thus, the present inventors recognised that existing methods to analyse HLA deregulation failed to accurately capture the full picture of HLA deregulation in cancer, and set out to provide an improved method.

The present disclosure provides a method for determining whether an HLA gene has deregulated expression in a sample from a subject, the method comprising analysing RNA sequencing data from the sample using a genomic reference sequence that is specific to HLA alleles identified to be present in the subject.

In describing the present invention, the following terms will be employed, and are intended to be defined as indicated below.

The terms “deregulated expression” or “transcriptional deregulation” refer to an altered level of expression of a specific HLA allele compared to a control level, and/or the presence of an exon skipping event in the HLA allele.

The human leukocyte antigen (HLA) system is a gene complex encoding the major histocompatibility complex (MHC) proteins in humans. These cell-surface proteins regulate the immune system in humans. The HLA gene complex resides on a 3 Mbp stretch within chromosome 6p21. HLA genes are highly polymorphic, which means that they have many different alleles, allowing them to fine-tune the adaptive immune system. At each HLA locus there may be thousands of possible alleles, for example as described in Shiina et al. Journal of Human Genetics (2009) 54, 15-39. As used herein, the term “HLA allele” is intended to refer to any allele at the HLA locus. HLA proteins corresponding to MHC class I (A, B, and C) present peptides from inside the cell. These peptides are produced from digested proteins that are broken down in the proteasomes. In general, the peptides are small polymers, about 8-11 amino acids in length. Foreign antigens presented by MHC class I attract killer T-cells that destroy cells. HLAs corresponding to MHC class II (DP, OM, DOA, DOB, DQ, and DR) present antigens from outside of the cell to T-lymphocytes. MHC class II molecules are normally found on antigen-presenting cells such as dendritic cells, mononuclear phagocytes, some endothelial cells, thymic epithelial cells, and B cells. These cells are important in initiating immune responses. These molecules may also be induced on other cells by interferon y. In the present disclosure, an HLA allele or gene may be a class I or class I HLA allele or gene. For example, an HLA allele may be an allele of HLA class I gene HLA-A, HLA-B or HLA-C. As another example, an HLA allele may be an allele of HLA class II gene HLA-DP, HLA-OM, HLA-DOA, HLA-DOB, HLA-DG or HLA-DR.

HLA alleles present in a subject or sample may be identified through a process called “HLA typing”, resulting in an “HLA profile”. In the methods described herein, an HLA profile for a subject is typically obtained by analysing sequencing data from a sample from the subject. The sequence data may be DNA sequence data. The sample may be the same sample in which HLA deregulation is being assessed, or another sample. For example, in the context of analysing a tumour sample from a subject, HLA typing may be performed using sequence data from a normal sample from the subject. Methods to do this are known in the art and include Optitype (Szolek et al., 2014), Polysolver (Shukla et al., 2015), and HLA-HD (Kawaguchi et al.). Alternatively, the HLA profile of an individual may be determined by HLA serotyping, HLA-phenotyping with single specific primer PCR (SSP-PCR) and/or HLA gene sequencing. Preferably, HLA typing comprises determining the identity of HLA alleles present in a subject including the allele group and specific HLA protein for each HLA allele. Advantageously, the HLA typing is performed using genomic sequence data (e.g. WES or WGS data) rather than expression (RNA) sequence data. For example, genomic sequence data (e.g. WES) from a normal sample from the subject investigated may be used for HLA typing. HLA typing may further comprise determining the presence of known nonsynonymous DNA substitutions in the coding region of a specific HLA protein identified, and/or the presence of known mutations in the noncoding region of a specific HLA protein identified.

A “patient-specific genomic reference” (also referred to herein as “patient specific reference sequence”, “patient-specific reference”, “subject-specific genomic reference”, “sample specific reference sequence” or simply “specific reference sequence”) refers to a genomic reference sequence that is specific to HLA alleles identified to be present in the patient. Such a reference may be obtained by identifying the HLA profile associated with a patient, subject or sample, and combining the genomic sequences corresponding to the alleles identified in the HLA profile. A “patient-specific transcriptomic reference” (also referred to herein as “subject-specific transcriptomic reference”, “sample specific transcriptomic reference sequence” or simply “specific transcriptomic reference sequence”) refers to a transcriptomic reference sequence that is specific to HLA alleles identified to be present in the patient. Such a reference may be obtained by identifying the HLA profile associated with a patient, subject or sample, and combining the transcriptomic sequences corresponding to the alleles identified in the HLA profile. The genomic and/or transcriptomic sequences of individual HLA alleles may be obtained from a database, such as e.g. the IPD-IMGT/HLA database. In the context of the present disclosure, a patient-specific reference sequence may be used to obtain an improved alignment of sequencing reads (whether DNA sequencing reads or RNA sequencing reads) from a sample, subject or patient. An alignment of sequencing reads to a patient specific reference sequence may be obtained by extracting reads that map or may map to the HLA locus, from a pre-aligned sequencing data set (in the form of e.g. a BAM file). The pre-aligned sequencing data may have been aligned to a standard reference, such as e.g. a standard reference genome (e.g. GRCh38 or GRCh37). An alignment of sequencing reads to a patient specific reference sequence may be obtained by extracting from sequencing data (which may be in the form of pre-aligned reads e.g. a BAM file), one or more of: (i) reads that contain a sequence that matches to a sequence from a set of target sequences, (ii) reads that map to chromosome 6 in the pre-aligned sequences, (iii) reads that are unmapped in the pre-aligned sequences, and (iv) reads that map to any contig in the pre-aligned sequences. The match may be an exact match. The set of target sequences may be a set of k-mers created from a reference sequence, such as a patient-specific reference sequence. The parameter k may be selected depending on the desired specificity of the selection step. Selection of sequences that have shorter matches will be more permissive (i.e. selecting more reads, some of which may not truly map to a patient-specific reference sequence), and conversely selection of sequences that have longer matches will be more restrictive (i.e. selecting fewer reads, potentially missing genuine reads that have mismatches due to e.g. mutations, sequencing errors or unknown splicing site). The present inventors have found k-mers of 30 nt to strike a good balance in this regard, for the purpose of the methods described herein. The extracted reads may be aligned (or re-aligned, if extracted from a pre-aligned sequence file) to the patient-specific reference sequence. This may produce a new aligned file, e.g. a new BAM file.

Antigens that are presented by the HLA system may be neoantigens. A neoantigen is a tumour-specific antigen which arises as a consequence of a mutation within a cancer cell. Thus, a neoantigen is not expressed by healthy (i.e. non-tumour cells). A neoantigen may be processed to generate distinct peptides which can be recognised by T cells when presented in the context of MHC molecules. As described herein, neoantigens may be used as the basis for cancer therapies, for example immunotherapies that target the neoantigen. An immunotherapy may be considered to target a neoantigen if it functions through recognition of the neoantigen by a component of the immune system. For example, a vaccine that is based on recognition of the neoantigen (e.g. including the neoantigen or a part thereof, or material enabling the expression of the neoantigen or part thereof) may be considered to be an immunotherapy that targets the neoantigen. Similarly, cell therapies that use cells that present or recognise the neoantigen may be considered to be immunotherapies that target the neoantigen. Methods for identifying or predicting neoantigens are known in the art, for example as described in McGranahan et al. 2016.

The binding of a neoantigen to a particular MHC molecule (encoded by a particular HLA allele) may be predicted using methods which are known in the art. Examples of methods for predicting MHC binding include those described by Lundegaard et al. (Nucleic Acids Res. 2008:W509-12.2008 & Bioinformatics. 2008 Jun. 1; 24(11):1397-8) and Shen et al. (Proteome Sci. 2013 Nov. 7; 11(Suppl 1):S15). For example, MHC binding of neoantigens may be predicted using the netMHC (Andreatta & Nielsen, Bioinformatics February 15; 32(4):511-7, 2016), netMHCpan (Jurtz et al, Journal of Immunology, ji1700893, 2017) or MHCflurry (O'Donnell et al., 2020) algorithms. A neoantigen may be considered to bind to a particular MHC molecule if a peptide sequence from said neoantigen is predicted to bind to said MHC molecule with a binding affinity that is below a suitable threshold. For example, the predicted binding affinity of the MHC molecule to a neoantigen peptide sequence may be below 500 nM. By “high affinity” may mean 0 to 50 nM binding affinity. A neoantigen peptide may be predicted to bind the MHC molecule with an intermediate affinity of 50 to 150 nM binding affinity, or low affinity of 150 to 500 nM binding affinity.

A “sample” as used herein may be a cell or tissue sample (e.g. a biopsy), a biological fluid, an extract (e.g. a DNA and/or RNA extract obtained from the subject), from which genomic material can be obtained for genomic analysis, such as genomic sequencing (whole genome sequencing, whole exome sequencing, targeted (also referred to as “panel” sequencing), transcriptomic sequencing (e.g. RNAseq, such as e.g. by targeted capture, polyA selection, etc.) or copy number array profiling. The sample may be a cell, tissue or biological fluid sample obtained from a subject (e.g. a biopsy). Such samples may be referred to as “subject samples”. In particular, the sample may be a blood sample, a tumour sample, a tumour-adjacent sample or a sample derived therefrom. The sample may be one which has been freshly obtained from a subject or may be one which has been processed and/or stored prior to genomic analysis (e.g. frozen, fixed or subjected to one or more purification, enrichment or extractions steps). In particular, the sample may be a cell or tissue culture sample. As such, a sample as described herein may refer to any type of sample comprising cells or genomic material derived therefrom, whether from a biological sample obtained from a subject, or from a sample obtained from e.g. a cell line. Unless context indicates otherwise, the term “genomic material” is used broadly to refer to both strictly speaking genomic material and transcriptomic material, i.e. any nucleic acid from which information about the genomic sequence present and/or expressed in the sample can be identified. The sample is preferably from a vertebrate (such as e.g. a vertebrate cell sample or a sample from a vertebrate subject), suitably from a mammalian (such as e.g. a mammalian cell sample or a sample from a mammalian subject, including in particular a model animal such as mouse, rat, etc.), preferably from a human (such as e.g. a human cell sample or a sample from a human subject). In embodiments, the sample is a sample obtained from a subject, such as a human subject. The terms “subject” and “patient” are used interchangeably herein. Further, the sample may be transported ad/or stored, and collection may take place at a location remote from the genomic sequence data acquisition (e.g. sequencing) location, and/or the computer-implemented method steps may take place at a location remote from the sample collection location and/or remote from the genomic data acquisition (e.g. sequencing) location (e.g. the computer-implemented method steps may be performed by means of a networked computer, such as by means of a “cloud” provider).

A “normal sample” or “germline sample” refers to a non-tumour sample, such as a blood sample, tissue sample (e.g. tumour adjacent normal tissue) or peripheral blood mononuclear cells from the subject. A “tumour sample” refers to a sample derived from or obtained from a tumour. The tumour may be a solid tumour or a non-solid or haematological tumour. The tumour sample may be a primary tumour sample, tumour-associated lymph node sample or sample from a metastatic site from the subject, or a tumour DNA containing bodily fluid (e.g. circulating tumour DNA). Such samples may comprise tumour cells, immune cells (such as e.g. lymphocytes), and other normal (non-tumour) cells. In the context of a tumour sample, the term “purity” refers to the proportion of cells in the sample that are tumour cells (also sometimes referred to as “cancer cell fraction” or “tumour fraction”), or to the equivalent proportion of cells in the case of a sample comprising genetic material derived from cells. In the context of samples comprising genetic material, a tumour fraction may be estimated using sequence analysis processes that attempt to deconvolute tumour and germline genomes such as e.g. ASCAT (Van Loo et al., 2010), ABSOLUTE (Carter et al., 2012), ichorCNA (Adalsteinsson et al., 2017), etc. A tumour sample may be a primary tumour sample, tumour-associated lymph node sample, or a sample from a metastatic site from the subject. A sample comprising tumour cells or genetic material derived from tumour cells may be a bodily fluid sample. Thus, the genetic material derived from tumour cells may be circulating tumour DNA or tumour DNA in exosomes. Instead or in addition to this, the sample may comprise circulating tumour cells. A sample may be a sample of cells, tissue or bodily fluid that has been processed to extract genetic material. Methods for extracting genetic material from biological samples are known in the art.

The term “sequence data” refers to information that is indicative of the presence and preferably also the amount of genomic material in a sample that has a particular sequence. Such information may be obtained using sequencing technologies, such as e.g. next generation sequencing (NGS, such as e.g. whole exome sequencing (WES), whole genome sequencing (WGS), whole transcriptome sequencing (also referred to as “RNAseq”), or sequencing of captured genomic loci (targeted or panel sequencing)), or using array technologies, such as e.g. copy number variation arrays, expression arrays, or other molecular counting assays. When NGS technologies are used, the sequence data may comprise a count of the number of sequencing reads that have a particular sequence. Sequence data may be mapped to a reference sequence, for example a reference genome, using methods known in the art (such as e.g. Bowtie (Langmead et al., 2009), NovoAlign (NovoCraftTechnologies), or STAR (Dobin et al., 2013)). Thus, counts of sequencing reads or equivalent non-digital signals may be associated with a particular location or locus (where the “location” refers to a location in the reference sequence to which the sequence data was mapped). The term “read depth” refers to a signal that is indicative of the amount of material in a sample that maps to a particular location. Such a signal may be obtained using sequencing technologies, such as e.g. next generation sequencing (NGS, such as e.g. WES, WGS, or panel sequencing), or using array technologies, such as e.g. copy number variation arrays, expression arrays. When NGS technologies are used, a read depth may be a read depth within the common sense of the word, i.e. a count of the number of sequencing reads mapping to a location. When array technologies are used, a read depth may be an intensity value associated with a particular location, which can be compared to a control to provide an indication of the amount of genomic material that maps to the particular location. The term “read depth profile” refers to a collection of read depth values relating to a plurality of genomic locations. For example, a read depth at a particular genomic location i may refer to the read depth at the base at position i in a reference sequence (genome or transcriptome), and a read depth profile may refer to the read depth for a plurality of positions i within one or more regions of interest. A reference sequence may be a genomic reference sequence or a transcriptomic reference sequence. A genomic reference sequence may include the complete genomic sequence of an organism or subject, or a part thereof, such as e.g. a chromosome or region of a chromosome (e.g. HLA locus). Thus, a genomic reference sequence may include coding and non coding sequences, including in particular introns and exons. A transcriptomic reference sequence may include the complete transcriptomic sequence of an organism or subject, including all known and/or predicted transcripts originating from the genome of the organism or subject. Thus, a transcriptomic reference sequence may not include non coding sequence, and in particular may not include introns.

As used herein “treatment” refers to reducing, alleviating or eliminating one or more symptoms of the disease which is being treated, relative to the symptoms prior to treatment. “Prevention” (or prophylaxis) refers to delaying or preventing the onset of the symptoms of the disease. Prevention may be absolute (such that no disease occurs) or may be effective only in some individuals or for a limited amount of time.

As used herein, the terms “computer system” includes the hardware, software and data storage devices for embodying a system or carrying out a method according to the above described embodiments. For example, a computer system may comprise a processing unit (such as a central processing unit (CPU) and/or graphical processing unit (GPU)), input means, output means and data storage, which may be embodied as one or more connected computing devices. Preferably the computer system has a display or comprises a computing device that has a display to provide a visual output display (for example in the design of the business process). The data storage may comprise RAM, disk drives or other computer readable media. The computer system may include a plurality of computing devices connected by a network and able to communicate with each other over that network. It is explicitly envisaged that computer system may consist of or comprise a cloud computer.

As used herein, the term “computer readable media” includes, without limitation, any non-transitory medium or media which can be read and accessed directly by a computer or computer system. The media can include, but are not limited to, magnetic storage media such as floppy discs, hard disc storage media and magnetic tape; optical storage media such as optical discs or CD-ROMs; electrical storage media such as memory, including RAM, ROM and flash memory; and hybrids and combinations of the above such as magnetic/optical storage media.

### Determining HLA Allele-Specific Transcriptional Deregulation in a Sample

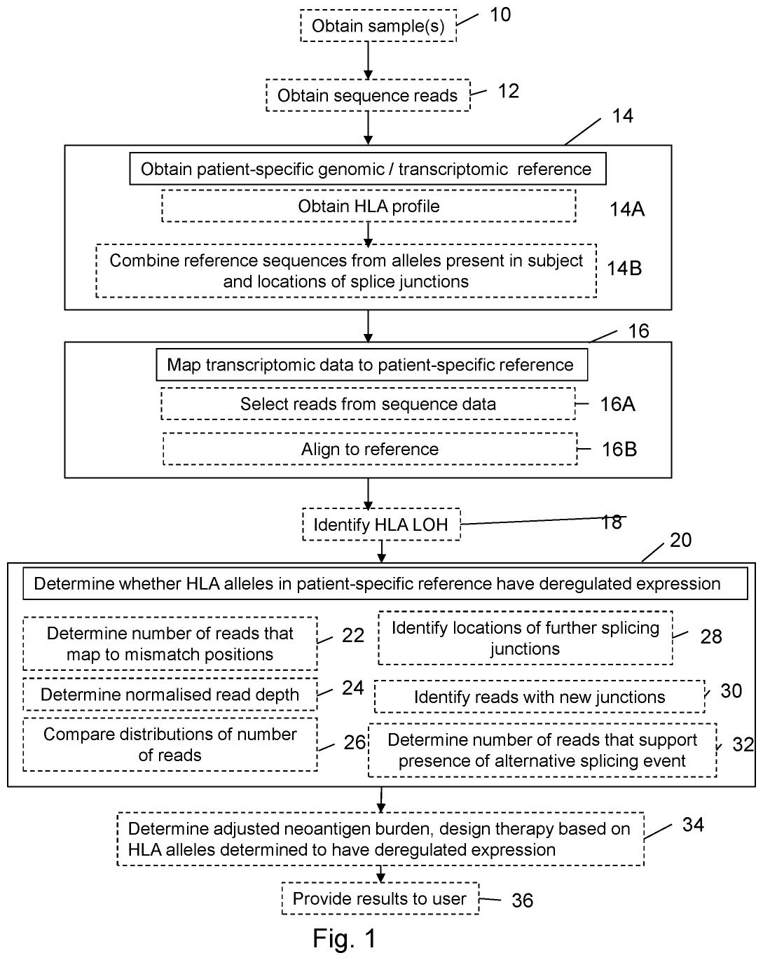

The present disclosure provides method for determining whether an HLA allele is transcriptionally deregulated in a sample. An illustrative method will be described by reference to FIG. 1. At optional step 10, one or more samples (such as e.g. a tumour sample and a normal sample) may be obtained from a subject. At optional step 12, sequence reads/read depth data may be obtained from the sample(s), for example by sequencing the genomic material in the sample(s) using one or more of whole exome sequencing, whole genome sequencing, whole transcriptome sequencing or panel sequencing. At least transcriptome sequence data is obtained from the sample for which HLA expression deregulation is to be determined (e.g. a tumour sample). Advantageously, transcriptome sequence data may also be obtained for a comparative sample, e.g. a matched normal sample. Further, genomic data may also be obtained from the sample for which HLA expression deregulation is to be determined, and/or from a normal/comparative sample (e.g. matched normal sample). At step 14, a patient-specific genomic and/or transcriptomic reference sequence is obtained. This may comprise step 14A of obtaining an HLA profile for the subject (e.g. using HLA-HD or Polysolver and the genomic data from the tumour or normal sample) specifying the HLA alleles present in the subject (e.g. based on DNA sequence data from a tumour and/or normal sample from the subject) and step 14B of combining reference sequences for the alleles determined to be present in the subject. Combining reference sequences for the alleles determined to be present in the subject may comprise obtaining the reference sequences from one or more databases. Combining reference sequences for the alleles determined to be present in the subject may comprise obtaining one or more partial reference sequences (e.g. from one or more databases) and completing partial reference sequences using reference sequences of an allele that has a high sequence similarity to a partial sequence. Optionally, a patient-specific genomic reference sequence and a patient-specific transcriptomic reference sequence are obtained. The genomic reference sequence may be used to detect alternative splicing events in one or more HLA alleles, as will be described further below. The genomic reference or the transcriptomic reference sequence may be used to detect transcriptional repression in one or more HLA alleles, as will be described further below. At step 16, the transcriptomic data (e.g. RNA sequence reads) from the sample for which deregulation is analysed is mapped to the patient-specific reference sequence (or to each of a patient-specific genomic reference sequence and a patient-specific transcriptomic reference sequence). This may comprise optional step 16A of selecting sequence reads from the sequence data. This may comprise extracting sequencing reads that map to the HLA region (e.g. by alignment to a standard reference genome or transcriptome) or to a region comprising the HLA region (e.g. a whole chromosome or a part thereof) in an alignment to a standard reference (e.g. a BMA file from a standard alignment process). This may instead or in addition comprise extracting sequencing reads that contain a sequence that matches a sequence in a list of target sequences comprising all k-mers in the patient-specific reference sequence. This may instead or in addition comprise extracting sequencing reads that do not map to any sequence of a standard reference sequence (i.e. not a patient-specific reference sequence), and/or extracting sequencing reads that do not map to a sequence that is not part of a chromosome in a standard reference sequence (e.g. one or more contigs of a standard reference sequence). Optionally, transcriptomic data from a comparative sample (e.g. matched normal sample) may also be aligned to the patient-specific reference using a similar process. A patient-specific genomic reference may be provided as a genomic sequence (e.g. a fasta file), and an indication of the locations of introns, exons, and/or corresponding splice junctions (e.g. a GTF file or other file comprising locations of introns, exons and optionally UTRs, from which locations of splice junctions can be inferred). In embodiments, aligning the transcriptomic data to a genomic reference sequence may comprise a first step of aligning the transcriptomic data to a patient-specific genomic reference sequence comprising a genomic sequence and an indication of the locations of a first set of introns, exons, and/or splice junctions (e.g. known locations from a reference sequence such as e.g. from a database) to identify a further set of splice junctions (which can be defined by the location of the splice junction of the location of the exon and intron between which the splice junction is located), and a second step of aligning the transcriptomic data to a patient-specific genomic reference sequence comprising the genomic sequence and an indication of the locations of the first and further sets of introns, exons, and/or splice junctions. At optional step 18, HLA loss of heterozygosity may be identified by quantifying allele specific copy numbers as described in McGranahan et al. (2017) and further below. At step 20, the aligned data is analysed to determine whether one or more HLA alleles in the subject has deregulated expression. This may comprise step 22 of determining the number of reads that map to mismatch positions between homologous alleles (allele-specific reads). Therefore, step 22 may comprise determining the number of reads that map uniquely to an allele that is analysed. This may be performed for each allele that is analysed. Optionally, a read depth for one or more alleles, which may be an optional read depth, may also be obtained at step 24. This may be obtained based on the number of reads that map uniquely to an allele and the number of reads that map to multiple alleles. Reads that map to multiple alleles, such as two alleles of a homologous allele pairs may be redistributed to the individual alleles. For example a final count for an allele may be obtained as the sum of the number of reads that map uniquely to the allele and a number of reads that map to both alleles of a homologous allele pair multiplied by the proportion of reads that map to each of the alleles in the homologous allele pair (e.g. reads that map uniquely to allele 1 divided by sum of reads that map uniquely to allele 1 and reads that map uniquely to allele 2). Identifying reads that map to a single allele may be performed based on the mismatch positions identified between homologous alleles. These are positions where the two homologous alleles have a different sequence (i.e. a different base). Reads that include such mismatch positions are expected to be allele specific. This step may be implicit in that it is also possible to simply check whether each read aligns to one or both alleles of a pair of homologous alleles. Step 20 may in embodiments comprise step 24 of determining a normalised read depth. In other embodiments, a normalised read depth for an allele is not obtained and instead the number of reads that map uniquely to an allele are compared between the sample and a control. The normalised read depth may be normalised by allele length and/or by total coverage. Normalisation for allele length may be performed by dividing the read count (also referred to as read depth) for an allele by the total length of the allele sequence. Normalisation for total allele coverage may be performed by dividing by the read count (optionally normalised by allele length) by a scaling factor that sums the total read count (optionally normalised by allele length) for multiple alleles, such as all alleles analysed (e.g. all class I alleles, all class II alleles, or all HLA alleles regardless of class). Normalisation for total allele coverage may be performed by dividing by the read count (optionally normalised by allele length) by a scaling factor that sums the total read count in the original sequence data (pre-aligned data prior to filtering). Step 20 may further comprise step 26 of comparing the numbers of reads that map to a respective one of a plurality of mismatch positions in the sample and the numbers of reads that map to a respective one of a plurality of mismatch positions in one or more control samples. Thus, a distribution of numbers of reads that map to mismatch positions (allele specific reads) for an allele may be compared to a corresponding control distribution. Instead or in addition to this, the normalised read depth for the sample obtained at step 24 may be compared to a control level. The control level may be the normalised read depth for the same allele in a normal sample such as a normal sample obtained from the same subject (matched normal sample). The control level may be the normalised read depth for the same allele in a plurality of normal samples such as a normal samples obtained from a plurality of subjects that have the allele. The comparison may involve any statistical test known in the art to compare point estimates and/or distributions of observations. Instead or in addition to steps 22-26, step 20 may comprise determining whether HLA alleles in the patient specific reference have deregulated expression, where the deregulated expression is an alternative splicing event. This may comprise step 28 of identifying the locations of further splicing junctions using the results of the alignment step 16, where further splicing junctions are splice junctions that were not included in an indication of the locations of introns/exons/splice junctions that was provided as an input to the alignment. This may be performed as described in Veeneman et al., 2015. may comprise identifying reads that support the existence of an alternative splicing event, such as e.g. exon skipping events, at step 30. These may be reads that map to non-adjacent exons (full exon skipping). For example reads that align to both exon 4 and exon 6 may support the skipping of exon 5. Alternatively, these may be reads that map to part of an exon, and the subsequent exon (exon end skipping). Alternatively, these may be reads that map to an exon, and part of the subsequent exon (exon start skipping). Alternatively, these may be reads that may to the end of an exon and the start of a subsequent intron (partial intron retention, intron start retained). Alternatively, these may be reads that may to an intron and the subsequent exon (partial intron retention, intron end retained). All such reads are reads that map to non-canonical combinations of exons/intron sequences. These may also be referred to as reads that contain a novel splice junction. The method may optionally further comprise step 32 of determining a number of reads that support the presence of a candidate alternative splicing (e.g. exon skipping) event. This may be the number of uniquely mapping reads that support the existence of an alternative splicing event as explained above. The method may further comprise comparing the number of reads that support the presence of a candidate alternative splicing event to a control value to determine whether an alternative splicing event is present. An alternative splicing event may be considered to be present if the number of such reads is above a predetermined threshold (e.g. 50 reads). Optionally, a ratio of novel-to-canonical transcripts may be determined for one or more alternative splicing events. This may be the ratio of the number of uniquely mapping reads containing a novel splice junction (reads that map to non-canonical combinations of exons/introns sequences as explained above) divided by the number of uniquely mapping reads containing the corresponding known splice junction. This may additionally optionally be scaled by diving by the purity of the tumour sample (tumour cell fraction) from which this was obtained. Such a scaled value may represent the fraction of cancer cells that carry the somatic alternative splicing event. It may be capped at 1. At optional step 34, the information in relation to altered expression obtained through optional steps 22-26 (transcriptional repression) or through steps 28-32 (presence of an alternative splicing event) may be used to determine an adjusted (or effective) neoantigen burden. This may be obtained as the number of mutations that give rise to a neoantigen that is predicted to bind to at least one allele that is not repressed, lost (i.e. subject to LOH) and/or with a relevant alternative splicing event (e.g. skipping of exon 3, 5 or 6 in the case of a class I allele). Instead or in addition to this, the information in relation to altered expression (obtained through optional steps 22-26) or the presence of an exon skipping event (obtained through optional steps 28-32) may be used to design a therapy that targets one or more neoantigens that are predicted to bind to at least one allele that is not repressed, lost (i.e. subject to LOH) and/or with a relevant exon skipping event (e.g. skipping of exon 3, 5 or 6 in the case of a class I allele). Allele-specific LOH may be identified as described in McGranahan et al. 2017 and WO2019/012296. At optional step 36, the determined normalised read depth, number of reads that support the presence of an exon skipping event and/or any value derived therefrom may be provided to a user, for example through a user interface. The value derived therefrom may include prognostic and/or diagnostic information, as described further below.

### Applications

The above methods find applications in a variety of clinical contexts, particularly in the context of cancer. For example, a key source of cytotoxic T cell response and immune activation in cancer is somatic mutations and their associated neoantigens, cancer cell specific mutations resulting in mutant peptides that elicit a T cell mediated immune response {Rooney, 2015; McGranahan, 2016}. A mutation can only engender a neoantigen if the associated mutant peptide is presented to T cells by human leukocyte antigen (HLA) molecules, and, as such, down-regulation or loss of HLA molecules can have important implications for immune evasion. Thus, when designing an immunotherapy that targets a neoantigen, it may be particularly important to determine whether the neoantigen to be targeted is predicted to be displayed by an HLA allele that has been lost or is deregulated in the subject to be treated. Further, a high tumour mutation burden (TMB) has been found to be associated with improved response to immune checkpoint blockade {Rizvi, 2015; Snyder 2014}. However, as a mutation can only engender a neoantigen and a T cell response if the associated mutant peptide is presented to T cells by human leukocyte antigen (HLA) molecules, assessment of TMB to predict response to therapy can be advantageously adjusted to take into account HLA deregulation.

Thus, also described herein is a method for determining whether a neoantigen is predicted to be presented by a tumour comprising the steps of: (i) identifying a neoantigen in a tumour; and (ii) determining whether said neoantigen is predicted to be presented by an HLA molecule encoded by an HLA allele that is not deregulated in said tumour, wherein step (ii) uses the methods described herein.

Also described herein is a method for identifying a target neoantigen for cancer therapy, comprising the steps of: (i) identifying a neoantigen in a tumour; (ii) determining whether said neoantigen is predicted to be presented by an HLA molecule encoded by an HLA allele that is deregulated in said tumour using a method described herein; and (iii) discounting neoantigens as targets which are predicted to be presented by an HLA molecule encoded by an HLA allele that is deregulated in said tumour. It is possible that neoantigens may be predicted to bind to more than one HLA allele, wherein one HLA allele may be deregulated in a tumour, but the other HLA allele is not lost in a tumour. In that case, the neoantigen may still be a target for cancer therapy and need not be discounted. As such, the methods above may comprise a step of discounting neoantigens as targets which are predicted to only be presented by HLA alleles that are deregulated in said tumour. Neoantigens may be retained as targets if they are predicted to be presented by at least one HLA allele that is not deregulated in a tumour. Target neoantigens identified according to the methods herein may be a target for any of the methods of treatment and corresponding uses as described herein.

The HLA allele that is not deregulated (or which is deregulated in the case where neoantigens are discounted) may have been determined to not be deregulated in at least one sample from a tumour. For example, the HLA allele may have been determined to not be deregulated in 2, 3, 4, 5, 6, 7, 8, 9 or 10 samples from said tumour. The samples may be taken from the same site (e.g. primary tumour) or from multiple sites (e.g. primary tumour and one or more metastases).

Neoantigens that are predicted to be presented by an HLA molecule encoded by an HLA allele which has been determined not to be deregulated in a tumour may represent a target for therapeutic or prophylactic intervention in the treatment or prevention of cancer in a subject. References herein to “neoantigens” are intended to include also peptides derived from neoantigens. A therapy targeting a neoantigen may comprise an active immunotherapy approach, such as administering an immunogenic composition or vaccine comprising a neoantigen to a subject. Alternatively, a passive immunotherapy approach may be taken, for example adoptive T cell transfer or B cell transfer, wherein a T or B cell or T and B cells which recognise a neoantigen are isolated from tumours, or other bodily tissues (including but not limited to lymph node, blood or ascites), expanded ex vivo or in vitro and readministered to a subject. Instead or in addition to this, an antibody which recognises a neoantigen may be administered to a subject. One skilled in the art will appreciate that if the neoantigen is a cell surface antigen, an antibody as referred to herein will recognise the neoantigen. Where the neoantigen is an intracellular antigen, the antibody will recognise the neoantigen peptide:MHC complex. As referred to herein, an antibody which “recognises” a neoantigen encompasses both of these possibilities. Thus, also described herein is a method of providing an immunotherapy for a subject, the method comprising: (i) identifying one or more neoantigens that are present in the subject; (ii) determining whether the one or more neoantigens are predicted to be presented by an HLA molecule encoded by an HLA allele that is deregulated in the subject using a method described herein; and (iii) providing an immunotherapy that targets a neoantigen of the one or more neoantigens that is predicted to be presented by an HLA molecule encoded by an HLA allele that is not deregulated in the subject.

Also disclosed herein is a method of treating or preventing cancer in a subject, comprising administering to said subject: (i) a neoantigen that is predicted to be presented by an HLA molecule encoded by an HLA allele which has been determined not to be deregulated in a tumour; (ii) an immune cell which recognises a neoantigen that is predicted to be presented by an HLA molecule encoded by an HLA allele which has been determined not to be deregulated in a tumour; or (iii) an antibody which recognises a neoantigen that is predicted to be presented by an HLA molecule encoded by an HLA allele which has been determined not to be deregulated in a tumour. The method may comprise determining whether an HLA molecule that is predicted to present the neoantigen is deregulated in the tumour.

Also described herein is a neoantigen that is predicted to be presented by an HLA molecule encoded by an HLA allele which has been determined not to be deregulated in a tumour for use in the treatment or prevention of cancer in a subject, and methods of providing such a neoantigen comprising determining whether an HLA molecule that is predicted to present the neoantigen is deregulated in the tumour. Thus, also described is the use of a neoantigen that is predicted to be presented by an HLA molecule encoded by an HLA allele which has been determined not to be deregulated in a tumour in the manufacture of a medicament for use in the treatment or prevention of cancer in a subject. The use may comprise determining whether an HLA molecule that is predicted to present the neoantigen is deregulated in the tumour. Also described is the use of a neoantigen that is predicted to be presented by an HLA molecule encoded by an HLA allele which has been determined not to be deregulated in a tumour in treating or preventing cancer in a subject. Also described herein is an immune cell, preferably a T cell which recognises a neoantigen that is predicted to be presented by an HLA molecule encoded by an HLA allele which has been determined not to be deregulated in a tumour for use in the treatment or prevention of cancer in a subject, and methods of providing such an immune cell comprising determining whether the HLA allele is deregulated in the tumour. Thus, also described is the use of an immune cell, preferably a T cell, which recognises a neoantigen that is predicted to be presented by an HLA molecule encoded by an HLA allele which has been determined not to be deregulated in a tumour in the manufacture of a medicament for use in the treatment or prevention of cancer in a subject. In a further alternative the invention provides the use of an immune cell, preferably a T cell, which recognises a neoantigen that is predicted to be presented by an HLA molecule encoded by an HLA allele which has been determined not to be deregulated in a tumour in treating or preventing cancer in a subject. References to “an immune cell” are intended to encompass cells of the immune system, for example T cells, NK cells, NKT cells, B cells and dendritic cells. In a preferred embodiment, the immune cell is a T cell, as discussed herein. Also described is an antibody which recognises a neoantigen that is predicted to be presented by an HLA molecule encoded by an HLA allele which has been determined not to be deregulated in a tumour for use in the treatment or prevention of cancer in a subject, and methods of providing such an antibody comprising determining whether the HLA allele is deregulated in the tumour. Thus, also described is the use of an antibody which recognises a neoantigen that is predicted to be presented by an HLA molecule encoded by an HLA allele which has been determined not to be deregulated in a tumour in the manufacture of a medicament for use in the treatment or prevention of cancer in a subject. Also described is the use of an antibody which recognises a neoantigen that is predicted to be presented by an HLA molecule encoded by an HLA allele which has been determined not to be deregulated in a tumour in treating or preventing cancer in a subject.

A “neoantigen” (or “neo-antigen”) is an antigen that arises as a consequence of a mutation within a cancer cell. Thus, a neoantigen is not expressed (or expressed at a significantly lower level) by normal (i.e. non-tumour) cells. A neoantigen may be processed to generate distinct peptides which can be recognised by T cells when presented in the context of MHC molecules. The neoantigen described herein may be caused by any mutation which alters a protein expressed by a cancer cell or its level of expression compared to the non-mutated protein expressed by a wild-type, healthy cell. For example, the mutated protein may be a translocation or fusion. A “mutation” refers to a difference in a nucleotide sequence (e.g. DNA or RNA) in a tumour cell compared to a healthy cell from the same individual. The difference in the nucleotide sequence can result in the expression of a protein which is not expressed by a healthy cell from the same individual. For example, the mutation may be a single nucleotide variant (SNV), multiple nucleotide variants, a deletion mutation, an insertion mutation, a translocation, a missense mutation or a splice site mutation resulting in a change in the amino acid sequence (coding mutation). The mutations may be identified by Exome sequencing, RNA-seq, whole genome sequencing and/or targeted gene panel sequencing and or routine Sanger sequencing of single genes. Suitable methods are known in the art. The neoantigen may be a clonal neoantigen. A “clonal neoantigen” (also sometimes referred to as “truncal neoantigen”) is a neoantigen that results from a mutation that is present in essentially every tumour cell in one or more samples from a subject (or that can be assumed to be present in essentially every tumour cell from which the tumour genetic material in the sample(s) is derived). A “sub-clonal” neoantigen is a neoantigen that results from a mutation that is present in a subset or a proportion of cells in one or more tumour samples from a subject (or that can be assumed to be present in a subset of the tumour cells from which the tumour genetic material in the sample(s) is derived). A neoantigen or mutation may be clonal in the context of one or more samples from a subject while not being truly clonal in the context of the entirety of the population of tumour cells that may be present in a subject (e.g. including all regions of a primary tumour and metastasis). The wording “essentially every tumour cell” in relation to one or more samples or a subject may refer to at least 70%, at least 75%, at least 80%, at least 85%, at least 90%, at least 91%, at least 92%, at least 93%, at least 94% at least 95%, at least 96%, at least 97%, at least 98%, or at least 99% of the tumour cells in the one or more samples or the subject. It will be appreciated that a determination that a mutation is ‘encoded within essentially every tumour cell’ refers to a statistical calculation and is therefore subject to statistical analysis and thresholds. Likewise, a determination that a clonal neoantigen is ‘expressed effectively throughout a tumour’ refers to a statistical calculation and is therefore subject to statistical analysis and thresholds. Expressed effectively in essentially every tumour cell, or essentially all tumour cells, means that the mutation is present in all tumour cells analysed in a sample, as determined using appropriate statistical methods. Methods to identify clonal neoantigens are known in the art and include the methods described in WO 2016/16174085, Landau et al. (2013), Roth et al. (2014), McGranahan et al. (2016).

Identifying one or more neoantigens may comprise identifying a plurality of neoantigens, such as e.g. between 2 and 1000 neoantigens. For example, the number of clonal neoantigens may be 2, 3, 4, 5, 6, 7, 8, 9, 10, 20, 50, 100, 150, 200, 250, 300, 350,400, 450, 500, 550, 600, 650, 700, 750, 800, 850, 900, 950 or 1000, for example the number of clonal neoantigens may be from 2 to 100. Thus, the methods as described herein may provide a plurality or population, i.e. more than one, of T cells wherein the plurality of T cells comprises a T cell which recognises a clonal neoantigen and a T cell which recognises a different clonal neoantigen. As such, the method provides a plurality of T cells which recognise different clonal neoantigens. In a preferred embodiment the number of clonal neoantigens recognised by the plurality of T cells is 2-1000. For example, the number of clonal neoantigens recognised may be 2, 3, 4, 5, 6, 7, 8, 9, 10, 20, 50, 100, 150, 200, 250, 300, 350, 400, 450, 500, 550, 600, 650, 700, 750, 800, 850, 900, 950 or 1000, for example the number of clonal neoantigens recognised may be from 2 to 100. The plurality of T cells may recognises the same clonal neoantigen. The neoantigen may be clonal or subclonal. The neoantigen may be the result of a single or multiple substitution mutation. The neoantigen may be the result of an indel, such as e.g. an insertion or a deletion of from 1 to 100 bases, for example 1 to 90, 1 to 50, 1 to 23 or 1 to 10 bases. The indel mutation may be a frameshift indel mutation. A frameshift indel mutation is a change in the reading frame of the nucleotide sequence caused by an insertion or deletion of one or more nucleotides. Such frameshift indel mutations may generate a novel open-reading frame which is typically highly distinct from the polypeptide encoded by the non-mutated DNA/RNA in a corresponding healthy cell in the subject. Frameshift mutations typically introduce premature termination codons (PTCs) into the open reading frame and the resultant mRNAs are targeted for nonsense mediated decay (NMD). A neoantigen peptide may comprise a cancer cell specific mutation (e.g. a non-silent amino acid substitution encoded by a single nucleotide variant (SNV)) at any residue position within the peptide. By way of example, a peptide which is capable of binding to an MHC class I molecule is typically 7 to 13 amino acids in length. As such, the amino acid substitution may be present at position 1, 2, 3, 4, 5, 6, 7, 8, 9, 10, 11, 12 or 13 in a peptide comprising thirteen amino acids. Depending on the application of the neoantigen peptide, longer peptides, for example 21-31 mers, may be used, and the mutation may be at any position, for example at the centre of the peptide, e.g. at positions 13, 14, 15 or 16 can also be used to stimulate both CD4 and CDS cells to recognise neoantigens.

Also described herein is a method for providing a T cell which is specific to a neoantigen that is predicted to be presented by an HLA molecule encoded by an HLA allele which has been determined not to be deregulated in a tumour, wherein said method comprises the following steps: i) identifying a neoantigen that is predicted to be presented by an HLA molecule encoded by an HLA allele that has been determined not to be deregulated in a tumour using a method described herein (which may comprise determine whether the HLA allele is deregulated in the tumour); and ii) providing a T cell or population of T cells which recognises said neoantigen. The T cell population may be expanded in order to increase the number of T cells which recognise or target a neoantigen that is predicted to be presented by an HLA molecule encoded by an HLA allele which has been determined not to be deregulated in a tumour. Expansion of T cells may be performed using methods which are known in the art. For example, T cells may be expanded by ex vivo culture in conditions which are known to provide mitogenic stimuli for T cells. By way of example, the T cells may be cultured with cytokines such as IL-2 or with mitogenic antibodies such as anti-CD3 and/or CD28. The T cells may be co-cultured with antigen-presenting cells (APCs), which may have been irradiated. The APCs may be dendritic cells or B cells. The dendritic cells may have been pulsed with peptides containing the identified neoantigen as single stimulants or as pools of stimulating neoantigen peptides. Expansion of T cells may be performed using methods which are known in the art, including for example the use of artificial antigen presenting cells (aAPCs), which provide additional co-stimulatory signals, and autologous PBMCs which present appropriate peptides. Autologous PBMCs may be pulsed with peptides containing neoantigens as discussed herein as single stimulants, or alternatively as pools of stimulating neoantigens. Thus, also described is a method for expanding a T cell population for use in the treatment of cancer in a subject, wherein the T cell population targets a neoantigen that is predicted to be presented by an HLA molecule encoded by an HLA allele which has been determined not to be deregulated in a tumour, the method comprising the steps of: a) providing a T cell population comprising a T cell which is capable of specifically recognising said neoantigen; and b) co-culturing the T cell population with a composition comprising the neoantigen. Expansion may be performed by co-culture of a T cell with a neoantigen and an antigen presenting cell. The antigen presenting cell may be a dendritic cell. The neoantigen may be a clonal neoantigen. The expansion may be a selective expansion of T cells which are specific for the neoantigen. Also described herein is a method for producing a composition comprising an antigen presenting cell and a neoantigen or a neoantigen peptide wherein said neoantigen or neoantigen peptide is one that is predicted to be presented by an HLA molecule encoded by an HLA allele which has been determined not to be deregulated in a tumour using the methods described herein. Said method may comprise the following steps: (a) identifying a neoantigen which is predicted to be presented by an HLA molecule encoded by an HLA allele that has been determined not to be deregulated in a tumour; and b) producing a composition comprising said neoantigen or neoantigen peptide and an antigen presenting cell. Also described is a composition comprising an antigen presenting cell, e.g. a dendritic cell, and a neoantigen or neoantigen peptide wherein said neoantigen or neoantigen peptide is one that is predicted to be presented by an HLA molecule encoded by an HLA allele which has been determined not to be deregulated in a tumour. The composition may be used in a method described herein, for example in methods of producing a T cell or T cell population or composition as discussed herein. Expansion may involve culturing the T cell population with IL-2 or an anti-CD3 and/or an CD28 antibody. The T cell population may be isolated from the patient to be treated, for example from a tumour sample obtained from said patient. The T cell population may comprise tumour infiltrating lymphocytes (TILs). A T cell composition is provided in which said T cell population is enriched with an increased number of T cells which target neoantigens that are predicted to be presented by an HLA molecule encoded by an HLA allele which has been determined not to be deregulated in a tumour compared with the initial T cell population isolated from the subject. Also provided is a T cell composition useful for the treatment of a cancer in a subject which comprises T cells selectively expanded to target neoantigens characteristic of the subject's cancer wherein said neoantigens are predicted to be presented by an HLA molecule encoded by an HLA allele which has been determined not to be deregulated in a tumour. A T cell composition as described herein may be enriched with T cells which are specific to neoantigens that are predicted to be presented by an HLA molecule encoded by an HLA allele which has been determined not to be deregulated in a tumour. In a T cell composition as described herein the expanded population of neoantigen-reactive T cells may have a higher activity than the population of T cells which have not been expanded, as measured by the response of the T cell population to restimulation with a neoantigen peptide. Activity may be measured by cytokine production, and wherein a higher activity is a 5-10 fold or greater increase in activity. A T cell, T cell population or T cell composition as described herein may be obtained or obtainable by any of the methods as described herein. A T cell, T cell population or T cell composition as described herein may be used in the treatment of cancer. Also described herein is a method for treating cancer in a subject comprising administering a T cell composition as described herein to the subject. Also described is a T cell composition as described herein for use in the manufacture of a medicament for the treatment of cancer. The method may comprise the following steps: (i) isolation of a T cell population from a sample from the subject; (ii) expansion of the T cell population which targets a neoantigen that is predicted to be presented by an HLA molecule encoded by an HLA allele which has been determined not to be deregulated in a tumour; and (iii) administering the T cell population from (ii) to the subject. The method may comprise the following steps: (i) isolation of a T cell from a sample from the subject; (ii) engineering the T cell to express a CAR or TCR which recognises said neoantigen as described herein to provide a T cell population which targets the neoantigen; and (iii) administering the T cell population from (ii) to the subject. Said T cells may be selectively expanded using a plurality of neoantigens, wherein each of said peptides comprises a different mutation. Said plurality may be from 2 to 250, from 3 to 200, from 4 to 150, or from 5 to 100 neoantigens, for example from 5 to 75 or from 15 to 50 neoantigens. A method of the disclosure may comprise firstly identifying a neoantigen that is predicted to be presented by an HLA molecule encoded by an HLA allele which has been determined not to be deregulated in a tumour, and then expanding a T cell population to target the neoantigen. Thus, Thus, also described is a method for providing a T cell population which targets a neoantigen that is predicted to be presented by an HLA molecule encoded by an HLA allele which has been determined not to be deregulated in a tumour, said method comprising the steps of: (a) identifying a neoantigen that is predicted to be presented by an HLA molecule encoded by an HLA allele which has been determined not to be deregulated in a tumour; and (b) expanding a population of T cells to provide a T cell population that targets the neoantigen. Following expansion, the resulting T cell population is enriched with an increased number of T cells which target neoantigens that are predicted to be presented by an HLA molecule encoded by an HLA allele which has been determined not to be deregulated in a tumour (for example, compared with the sample isolated from the subject).

Any method described herein may comprise the steps of identifying one ore more neoantigens, predicting one or more HLA alleles that are likely to present a neoantigen peptide associated with each of the one or more neoantigens, and determining whether the one or more HLA alleles are deregulated in the tumour using a method as described herein. Such a method may further comprise selecting one or more neoantigens or neoantigen peptides that are likely to be presented by at least one HLA allele that is determined not to be deregulated in the tumour.

Thus, also described is a T cell which recognises a neoantigen that is predicted to be presented by an HLA molecule encoded by an HLA allele which has been determined not to be deregulated in a tumour. In a further aspect the invention relates to a population of T cells which recognise a neoantigen that is predicted to be presented by an HLA molecule encoded by an HLA allele which has been determined not to be deregulated in a tumour or a population of T cells as described herein. A population may be a plurality or population, i.e. more than one, of T cells wherein the plurality of T cells comprises a T cell which recognises a neoantigen that is predicted to be presented by an HLA molecule encoded by an HLA allele which has been determined not to be deregulated in a tumour, and a T cell which recognises a different neoantigen that may be presented by an HLA which has been determined not to have been lost in a tumour. As such, also described is a plurality of T cells which recognise different neoantigens. Different T cells in the plurality or population may have different TCRs which recognise the same neoantigen. The T cell population may be all or primarily composed of CDS+ T cells, or all or primarily composed of a mixture of CD8+ T cells and CD4+ T cells or all or primarily composed of CD4+ T cells. The T cell population may be generated from T cells isolated from a subject with a tumour. For example, the T cell population may be generated from T cells in a sample isolated from a subject with a tumour. The sample may be a tumour sample, a peripheral blood sample or a sample from other tissues of the subject. The T cell population may be generated from a sample from the tumour in which the neoantigen is identified. In other words, the T cell population is isolated from a sample derived from the tumour of a patient to be treated. the T cell population comprises tumour infiltrating lymphocytes (TILs). T cells may be isolated using methods which are well known in the art. For example, T cells may be purified from single cell suspensions generated from samples on the basis of expression of CD3, CD4 or CDS. T cells may be enriched from samples by passage through a Ficoll-paque gradient. The present disclosure also provides a method for providing a T cell population which targets a neoantigen in a tumour from a subject which comprises the steps of: i) isolating a T cell or population of T cells from a sample isolated from the subject; and ii) expanding the T cell or population of T cells to increase the number or relative proportion of T cells that target neoantigens that are predicted to be presented by an HLA molecule encoded by an HLA allele which has been determined not to be deregulated in a tumour. The T cell population that is produced in accordance with the present disclosure will have an increased number or proportion of T cells that target one or more neoantigens that are predicted to be presented by an HLA molecule encoded by an HLA allele which has been determined not to be deregulated in a tumour. For example, the T cell population of the invention will have an increased number of T cells that target a neoantigen that is predicted to be presented by an HLA molecule encoded by an HLA allele which has been determined not to have been lost in a tumour compared with the T cells in the sample isolated from the subject. The T cell population according to the invention may have at least about 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95 or 100% T cells that target a neoantigen that is predicted to be presented by an HLA molecule encoded by an HLA allele which has been determined not to be deregulated in a tumour. For example, the T cell population may have about 0.2%-5%, 5%-10%, 10-20%, 20-30%, 30-40%, 40-50%, 50-70% or 70-100% T cells that target a neoantigen that is predicted to be presented by an HLA molecule encoded by an HLA allele which has been determined not to be deregulated in a tumour. The T cell population may have at least about 1, 2, 3, 4 or 5% T cells that target a neoantigen that is predicted to be presented by an HLA molecule encoded by an HLA allele which has been determined not to be deregulated in a tumour, for example at least about 2% or at least 2% T cells that target a neoantigen that is predicted to be presented by an HLA molecule encoded by an HLA allele which has been determined not to be deregulated in a tumour. An expanded population neoantigen-reactive T cells may have a higher activity than a population of T cells not expanded, for example, using a neoantigen peptide. Reference to “activity” may represent the response of the T cell population to restimulation with a neoantigen peptide, e.g. a peptide corresponding to the peptide used for expansion, or a mix of neoantigen peptides. Suitable methods for assaying the response are known in the art. For example, cytokine production may be measured (e.g. IL2 or IFNy production may be measured). The reference to a “higher activity” includes, for example, a 1-5, 5-10, 10-20, 20-. 50, 50-100, 100-500, 500-1000-fold increase in activity. In one aspect the activity may be more than 1000-fold higher. A T cell as described herein may be an engineered T cell. The neoantigen specific T cell described herein may express a chimeric antigen receptor (CAR) or a T cell receptor (TCR) which specifically binds a neoantigen or a neoantigen peptide, or an affinity-enhanced T cell receptor (TCR) which specifically binds a neoantigen or a neoantigen peptide (as discussed further hereinbelow). For example, the T cell may express a chimeric antigen receptor (CAR) or a T cell receptor (TCR) which specifically binds to a neoantigen or a neoantigen peptide (for example an affinity enhanced T cell receptor (TCR) which specifically binds to a neoantigen or a neoantigen peptide). CARs are proteins which, in their usual format, graft the specificity of a monoclonal antibody (mAb) to the effector function of a T-cell. Their usual form is that of a type I transmembrane domain protein with an antigen recognizing amino terminus, a spacer, a transmembrane domain all connected to a compound endodomain which transmits T-cell survival and activation signals. The most common form of these molecules use single-chain variable fragments (scFv) derived from monoclonal antibodies to recognize a target antigen. The scFv is fused via a spacer and a transmembrane domain to a signaling endodomain. Such molecules result in activation of the T-cell in response to recognition by the scFv of its target. When T cells express such a CAR, they recognize and kill target cells that express the target antigen. Several CARs have been developed against tumour associated antigens, and adoptive transfer approaches using such CAR-expressing T cells are currently in clinical trial for the treatment of various cancers. Methods for generating TCRs and affinity enhanced TCRs are known in the art. Affinity enhanced TCRs are TCRs with enhanced affinity for a peptide-MHC complex (including e.g. the isolation of TCR genes that encode TCRs from patient samples (e.g. patient peripheral blood or TIIs) and the improvement of TCR affinity for a peptide-MHC complex via modification of TCR sequences (e.g. by in vitro mutagenesis and selection of enhanced affinity (or affinity matured) TCRs). Methods of introducing such TCR genes into T cells are known in the art. Methods of identifying optimal-affinity TCRs involving the immunisation of antigen-negative humanised transgenic mice which have a diverse human TCR repertoire (e.g. TCR/MHC humanised mice such as ABabDII mice) with antigen, and isolation of antigen-specific TCRs from such immunised transgenic mice are also known in the art. T cells may bear high affinity TCRs, and hence affinity enhancement may not be necessary. High affinity TCRs may be isolated from T cells from a subject and may not require affinity enhancement. Candidate T cell clones capable of binding a neoantigen peptide as described herein may be identified using MHC multimers comprising the neoantigen peptide, for example. Identified TCRs and/or CARs which specifically target a neoantigen peptide or neoantigen may be expressed in autologous T cells from a subject using methods which are known in the art, for example by introducing DNA or RNA coding for the TCR or CAR by one of many means including transduction with a viral vector, transfection with DNA or RNA. Also disclosed is a method for treating cancer in a subject which comprises administering a T cell or T cell population as described herein to the subject. The method may comprise the following steps: (i) isolation of a T cell-containing sample from the subject; (ii) expansion of a T cell population which targets an neoantigen as defined herein; and (iii) administering the cells from (ii) to the subject. The T cell may be engineered to express a CAR or affinity-enhanced TCR as described herein. The disclosure also provides a method of treating a patient who has cancer comprising administering to said patient a T cell or T cell population as defined herein. The neoantigen, T cell or T cell population may have been identified or produced according to any method described herein. The expansion may be ex vivo or in vitro, and may be performed by methods known in the art. Also described is a composition comprising an antigen presenting cell, and a neoantigen or neoantigen peptide as described herein. The antigen presenting cells may have been pulsed or loaded with said peptide. Also described is a T cell composition which comprises a population of neoantigen-specific T cells as described herein, wherein said population of neoantigen-specific T cells are produced by co-culturing T cells with antigen presenting cells which present neoantigen peptides. The antigen presenting cell may be a dendritic cell. The antigen presenting cell is irradiated. In one aspect the antigen presenting cell is a cell capable of presenting the relevant peptide, for example in the correct HLA context. Such a cell may be an autologous activated PBMC expressing an autologous HLA molecule, or a non autologous cell expressing an array of matched HLAs. In one aspect the artificial antigen presenting cell is irradiated. T cells may also be enriched by initial stimulation of TI Ls with neoantigens in the presence or absence of exogenous APCs followed by polyclonal stimulation and expansion with cytokines such as IL-2 or with mitogenic antibodies such as anti-CD3 and/or CD28. Such methods are known in the art. Also described is an antibody which recognises a neoantigen that is predicted to be presented by an HLA molecule encoded by an HLA allele which has been determined not to be deregulated in a tumour. “Antibody” (Ab) includes monoclonal antibodies, polyclonal antibodies, multispecific antibodies (e.g., bispecific antibodies), and antibody fragments that exhibit the desired biological activity. The term “immunoglobulin” (Ig) may be used interchangeably with “antibody”. Also described is an immunogenic composition, or vaccine, comprising a neoantigen or neoantigen peptide that may be presented by an HLA which has been determined not to be deregulated in a tumour. The immunogenic composition or vaccine may be used in any method of treating or preventing cancer according to the invention. As such, also described is a method of treating or preventing cancer in a subject comprising administering to the subject an immunogenic composition or vaccine according to the disclosure. By “immunogenic composition” is meant a composition that is capable of inducing an immune response in a subject. The immunogenic composition may be a vaccine composition. By “vaccine composition” is meant a composition that is capable of inducing an immune response in a subject that has a therapeutic or prophylactic effect on the condition to be treated. The immunogenic composition or vaccine may comprise more than one neoantigen or neoantigen peptide. The immunogenic composition or vaccine may comprise more than one different neoantigen or neoantigen peptide, for example 2, 3, 4, 5, 6, 7, 8, 9 or 10 different neoantigens or neoantigen peptides. The neoantigen may also be in the form of a protein. The immunogenic composition or vaccine may comprise a polypeptide which comprises an neoantigen as defined herein. The immunogenic composition or vaccine may comprise more than one different polypeptide each comprising a neoantigen, for example 2, 3, 4, 5, 6, 7, 8, 9 or 10 different polypeptides. The immunogenic composition or vaccine may lead to generation of an immune response in the subject. An “immune response” which may be generated may be humeral and/or cell-mediated immunity, for example the stimulation of antibody production, or the stimulation of cytotoxic or killer cells, which may recognise and destroy (or otherwise eliminate) cells expressing antigens corresponding to the antigens in the vaccine on their surface. The term “stimulating an immune response” thus includes all types of immune responses and mechanisms for stimulating them and encompasses stimulating CTLs which forms a preferred aspect of the invention. Preferably the immune response which is stimulated is cytotoxic CDS+ T cells and helper CD4+ T Cells. The extent of an immune response may be assessed by markers of an immune response, e.g. secreted molecules such as IL-2 or IFNy or the production of antigen specific T cells. In addition, a neoantigen may be delivered in the form of a cell, such as an antigen presenting cell, for example a dendritic cell. The antigen presenting cell such as a dendritic cell may be pulsed or loaded with the neoantigen or neoantigen peptide or genetically modified (via DNA or RNA transfer) to express one, two or more neoantigens or neoantigen peptides, for example 2, 3, 4, 5, 6, 7, 8, 9 or 10 neoantigens or neoantigen peptides. Methods of preparing dendritic cell immunogenic compositions or vaccines are known in the art. Alternatively, DNA or RNA encoding one or more neoantigen, or peptide or protein derived therefrom as defined herein may be used in the immunogenic composition or vaccine, for example by direct injection to a subject. For example, DNA or RNA encoding 2, 3, 4, 5, 6, 7, 8, 9 or 10 neoantigens, or peptide or protein derived therefrom. The one or more neoantigen or neoantigen peptide may be delivered via a bacterial or viral vector containing DNA or RNA sequences which encode one or more neoantigens or neoantigen peptides. Also described is a cell expressing a neoantigen as defined herein, or a part thereof, on its surface, or a population thereof, which cell is obtainable (or obtained) by any of the methods herein. Such a cell may be used for treating or preventing cancer. The disclosure therefore further provides a cell expressing an neoantigen as defined herein or neoantigen peptide on its surface (or intracellularly), or a population of such cells, which cell or population is obtainable (or obtained) by methods as defined herein. The cell may be an antigen presenting cell such as a dendritic cell. the invention provides a method for producing an immunogenic composition or vaccine comprising an neoantigen peptide or neoantigen, wherein said neoantigen may be presented by an HLA molecule encoded by an HLA allele that has been determined not to be deregulated in a tumour, said method comprising the steps of: (a) identifying a neoantigen that is predicted to be presented by an HLA molecule encoded by an HLA allele that has been determined not to be deregulated in a tumour; and (b) producing an immunogenic composition or vaccine with said neoantigen peptide or neoantigen protein. Producing the vaccine may involve preparing a dendritic cell vaccine, wherein said dendritic cell presents a neoantigen or neoantigen peptide as defined herein. Also described is a method for producing an immunogenic composition or vaccine comprising a DNA or RNA molecule encoding a neoantigen peptide or neoantigen, said method comprising the steps of: (a) identifying a neoantigen that is predicted to be presented by an HLA molecule encoded by an HLA allele that has been determined not to be deregulated in a tumour; and (b) producing a DNA or RNA molecule encoding the neoantigen peptide or neoantigen; and (c) producing an immunogenic composition or vaccine with said DNA or RNA molecule.

The methods of the present disclosure find use in multiple clinical contexts where different diagnosis and/or prognosis may be associated with different tumour mutational burdens in said subjects. Thus, the disclosure also relates to methods for stratifying a population of subjects according to their tumour mutational burden, the method comprising (i) identifying a plurality of neoantigens in each subject in the population to obtain a first tumour mutational burden for each subject; (ii) carrying out the methods described herein one or more samples (e.g. tumour samples) from each of the subjects in the population to identify whether ne or more HLA alleles are deregulated in each subjects; (iii) adjusting the first tumour mutational burden for each subject to exclude neoantigens predicted to bind to an HLA allele that has been determined at step (ii) to be deregulated in a tumour from the subject. For example, the subjects may be separated between a “high (adjusted) tumour mutational burden” group and a “low (adjusted) tumour mutational burden” group, depending on the adjusted TMB estimated for the one or more tumour samples from the subjects. This may finds particular application to the identification of patients for personalised medicine and/or clinical trials. Patients classified as low adjusted TMB are expected to be less responsive to immunotherapy such as using checkpoint inhibitors, compared to patients classified as high adjusted TMB. Thus, the latter may be more likely to benefit from immunotherapy, whereas the former may be more likely to benefit from alternative therapeutic options. Instead or in addition to this, patients classified as having low TMB may have poorer survival prognosis than patients classified as high TMB. Thus, the methods of the present disclosure also finds uses in classifying a subject that has been diagnosed with cancer between at least two groups that have different prognosis and/or predicted sensitivity to one or more immunotherapies.

Thus, the disclosure also provides a method of providing a prognosis for a subject that has been diagnosed as having cancer, the method comprising (i) identifying one or more neoantigens in the subject to obtain a first tumour mutational burden for the subject; (ii) carrying out the methods described herein on one or more samples (e.g. tumour samples) from the subject to determine whether one or more HLA alleles are deregulated in the subject; (iii) adjusting the first tumour mutational burden for the subject to exclude neoantigens predicted to bind to an HLA allele that has been determined at step (ii) to be deregulated in a tumour from the subject; and (iv) classifying the subject between a plurality of groups associated with a different prognosis based on the adjusted TMB obtained at step (iii). The plurality of groups may comprise a first group associated with a higher adjusted TMB than a second group, wherein the first group has a better prognosis than the second group.

The present inventors have further identified that HLA allele deregulation, and in particular HLA expression, was indicative of prognosis, such as e.g. disease free survival. Thus, the disclosure also provides a method of providing a prognosis for a subject that has been diagnosed as having cancer, the method comprising (i) carrying out the methods described herein on one or more samples (e.g. tumour samples) from the subject to determine whether one or more HLA alleles are deregulated in the subject (in particular this may comprise determining the level of expression of the one or more alleles in the one or more samples and optionally calculating a total level of expression across all alleles); (ii) predicting survival based on the results of step (i). Predicting survival may comprise comparing the results of step (i) to one or more reference values, for example reference values corresponding to one or more cohorts of patients that have a known survival. For example, predicting survival may comprise comparing the results of step (i) to corresponding results obtained from subjects in a first cohort of patients that have a first prognosis, and a second cohort of patients that have a second, better prognosis.

Whether a prognosis is considered good or poor for a tumour sample that satisfies one or more predetermined criteria may vary between cancers and stage of disease. In general terms a good prognosis is one where the overall survival (OS), disease free survival (DFS) and/or progression-free survival (PFS) is longer than that of a comparative group or value, such as e.g. the average for that stage and cancer type, or the average for a comparative group of cancers that do not satisfy one or more criteria. A prognosis may be considered poor if OS, DFS and/or PFS is lower than that of a comparative group or value, such as e.g. the average for that stage and type of cancer, or the average for a comparative group of cancers that do not satisfy one or more criteria. Thus, in general terms, a “good prognosis” is one where survival (OS, DFS and/or PFS) and/or disease stage of an individual patient can be favourably compared to what is expected in a population of patients within a comparable disease setting. Similarly, a “poor prognosis” is one where survival (OS, DFS and/or PFS) of an individual patient is lower (or disease stage worse) than what is expected in a population of patients within a comparable disease setting.

TMB (tumour mutational burden—the number of somatic mutations present in a tumour), and the related metric of neoantigen burden (the number of somatic mutations that lead to a neoantigen present in a tumour—i.e. somatic mutations that lead to expression of a protein or peptide that is not expressed in normal cells) has also been found to be associated with response to checkpoint inhibition therapy (CPI). Further, it has been shown that correcting TMB to account for LOH of the HLA allele could improve the prediction of response to PD-(L)1 blockade in NSCLC (Shim et al., 2020). The present inventors have identified that taking into account other modes of deregulation when determining the TMB or neoantigen burden could further improve this prediction. Thus, the disclosure also provides a method of identifying a therapy for a subject that has been diagnosed as having cancer, the method comprising (i) identifying one or more neoantigens in the subject to obtain a first tumour mutational burden or neoantigen count for the subject; (ii) carrying out the methods described herein on one or more samples (e.g. tumour samples) from the subject to determine whether one or more HLA alleles are deregulated in the subject; (iii) adjusting the first tumour mutational burden or neoantigen count for the subject to exclude neoantigens predicted to bind to an HLA allele that has been determined at step (ii) to be deregulated in a tumour from the subject; and (iv) classifying the subject between a plurality of groups associated with a different responses to CPI therapy based on the adjusted TMB or neoantigen count obtained at step (iii). The adjusted TMB or neoantigen count may exclude any mutations that leads to a peptide that is predicted to bind only to alleles that are determined to be deregulated in a tumour from the subject. The adjusted TMB or neoantigen count may exclude any mutations that leads to a peptide that is predicted to bind to alleles that are determined to be lost and/or repressed and/or with skipping of exon 3 in a tumour from the subject. Step (i) may comprise identifying a plurality of somatic mutations present in the subject to identify a tumour mutational burden. Step (i) may further comprise identifying neoantigens as mutations of the plurality of somatic mutations that lead to a protein or peptide that is not expressed in normal cells. Method for predicting the binding of neoantigens (or any candidate peptides) to HLA alleles are known in the art and include e.g. NetMHC, NetMHCpan, and MHCflurry. The plurality of groups may comprise a first group associated with a higher adjusted TMB/neoantigen count than a second group, wherein the first group has a better response to CPI therapy than the second group. The method may further comprise selecting the subject for treatment with CPI therapy if the subject is classified in the first group. The method may further comprise selecting the subject for treatment with an alternative therapy if the subject is not classified in the first group.

Immune checkpoint molecules include both inhibitory and activatory molecules, and interventions may apply to either or both types of molecule. Immune checkpoint inhibitors include, but are not limited to, PD-1 inhibitors, PD-L 1 inhibitors, Lag-3 inhibitors, Tim-3 inhibitors, TIGIT inhibitors, BTLA inhibitors and CTLA-4 inhibitors, for example. Co-stimulatory antibodies deliver positive signals through immune-regulatory receptors including but not limited to ICOS, C0137, C027 OX-40 and GITR. In a preferred embodiment the checkpoint inhibitor is a CTLA-4 inhibitor. Examples of suitable immune checkpoint interventions which prevent, reduce or minimize the inhibition of immune cell activity include pembrolizumab, nivolumab, atezolizumab, durvalumab, avelumab, tremelimumab and ipilimumab.

The subject is preferably a human patient. The cancer may be ovarian cancer, breast cancer, endometrial cancer (uterus/womb cancer), kidney cancer (renal cell), lung cancer (small cell, non-small cell and mesothelioma), brain cancer (gliomas, astrocytomas, glioblastomas), melanoma, merkel cell carcinoma, clear cell renal cell carcinoma (ccRCC), lymphoma, gastrointestinal cancer (e.g. colorectal cancer), small bowel cancers (duodenal and jejunal), leukemia, pancreatic cancer, hepatobiliary tumours, liver cancer (e.g. hepatocellular carcinoma), germ cell cancers, prostate cancer, head and neck cancers, bladder cancer, thyroid cancer, oesophagal cancer, melanoma (e.g. uveal melanoma), cutaneous squamous cell carcinoma and sarcomas. The cancer may be lung cancer, such as lung adenocarcinoma or non small cell lung cancer (NSCLC).

### Systems

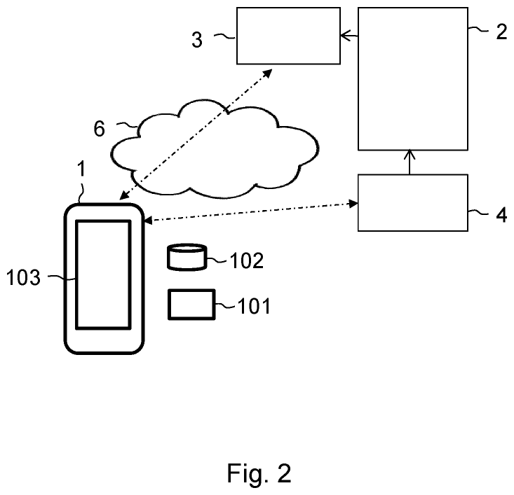

FIG. 2 shows an embodiment of a system for determining HLA allele-specific transcriptional 20 deregulation in a sample and/or for providing a prognosis or treatment recommendation based at least in part on the lymphocyte fraction, according to the present disclosure. The system comprises a computing device 1, which comprises a processor 101 and computer readable memory 102. In the embodiment shown, the computing device 1 also comprises a user interface 103, which is illustrated as a screen but may include any other means of conveying information to a user such as e.g. through audible or visual signals. The computing device 1 is communicably connected, such as e.g. through a network 6, to sequence data acquisition means 3, such as a sequencing machine, and/or to one or more databases 2 storing read depth data. The one or more databases may additionally store other types of information that may be used by the computing device 1, such as e.g. reference sequences, parameters, etc. The computing device may be a smartphone, tablet, personal computer or other computing device. The computing device is configured to implement a method for determining HLA allele-specific transcriptional deregulation in a sample, as described herein. In alternative embodiments, the computing device 1 is configured to communicate with a remote computing device (not shown), which is itself configured to implement a method for determining HLA allele-specific transcriptional deregulation in a sample, as described herein. In such cases, the remote computing device may also be configured to send the result of the method of determining the lymphocyte fraction to the computing device. Communication between the computing device 1 and the remote computing device may be through a wired or wireless connection, and may occur over a local or public network such as e.g. over the public internet or over WiFi. The sequence data acquisition means may be in wired connection with the computing device 1, or may be able to communicate through a wireless connection, such as e.g. through WiFi, as illustrated. The connection between the computing device 1 and the sequence data acquisition means 3 may be direct or indirect (such as e.g. through a remote computer). The sequence data acquisition means 3 are configured to acquire read depth data from nucleic acid samples, for example genomic DNA samples extracted from cells and/or tissue samples. In some embodiments, the sample may have been subject to one or more preprocessing steps such as RNA purification, fragmentation, library preparation, target sequence capture (such as e.g. panel sequence capture). Preferably, the sample has not been subject to amplification, or when it has been subject to amplification this was done in the presence of amplification bias controlling means such as e.g. using unique molecular identifiers. Any sample preparation process that is suitable for use in the determination of a an expression profile (whether whole transcriptome or sequence specific) may be used within the context of the present disclosure. The sequence data acquisition means is preferably a next generation sequencer. The sequence data acquisition means 3 may be in direct or indirect connection with one or more databases 2, on which sequence data (raw or partially processed) may be stored.

The following is presented by way of example and is not to be construed as a limitation to the scope of the claims.

### EXAMPLES

Emerging data have highlighted the importance of considering cancer evolution in the context of a predatory immune microenvironment. Key mediators of the cytotoxic T cell response in cancer are neoantigens, which are cancer cell specific alterations that result in mutant peptides that elicit a T cell mediated, human leukocyte antigen (HLA)-restricted, immune response. A mutation can only result in a neoantigen if the associated mutant peptide is presented on HLA molecules to the T cell receptor. A mutation can only result in a neoantigen if the associated mutant peptide is presented to T cells by human leukocyte antigen (HLA) molecules, and, as such, down-regulation or loss of HLA molecules can have important implications for immune evasion.

Disruption to antigen-presenting machinery such as e.g. by loss and/or down regulation of the HLA class I and II genes has been shown to occur across many cancer types (Schaafsma et al., 2021, McGranahan et al., 2017, Momburg et al., 1986). The inventors' previous work has shown HLA loss of heterozygosity (LOH), whereby one of the parental alleles is somatically lost during cancer evolution, occurs in 40% of non-small cell lung cancer (NSCLC) primary tumours (McGranahan et al., 2017). A pan cancer study suggested that transcriptomic down regulation of the HLA class I and II genes occurs frequently (Schaafsma et al., 2021). Their work has also shown that cancer subclones with HLA LOH harbour more non-synonymous mutations compared to their sister subclone without HLA LOH. However, subtle transcriptomic alterations in the HLA genes, such as alternative splicing events and allele-specific repression, have been poorly studied in cancer. Alternative splicing of the HLA genes has been reported in non-cancer tissue and in some cancer cell lines, which can result in a non-functional HLA molecule, or in the case of exon 5 skipping, soluble isoforms of the HLA molecule (Tijssen et al., 2000, Gerritsen 2016, Dubois et al., 2004, Reinders et al., 2005). Therefore, understanding HLA expression in adjacent normal tissue is of critical importance when attributing any change in HLA expression as a tumour specific phenomenon.

The HLA locus is located on one of the most polymorphic regions of the human genome {Horten, 2008}. As such, standard approaches to explore transcript expression and allele specific genomic loss cannot be used as a large fraction of HLA sequencing reads will fail to map to a standard reference {Shukla, 2016; McGranahan, 2017}. Thus, accurate quantification of HLA genomic and transcriptomic events benefits from a patient specific approach, taking into account germline HLA haplotypes. The inventors previously developed the first patient specific approach to evaluate HLA allele specific copy number in cancer samples, called LOHHLA (Loss of Heterogyzosity in Human Leukocyte Antigen), {McGranahan, 2017}. Building upon this tool, the inventors developed a new method to enable analysis of allele specific HLA expression and copy number in tumour samples at both the class I and class II loci. The examples below focus primarily on class I genes but the same principles are applicable to class II genes.

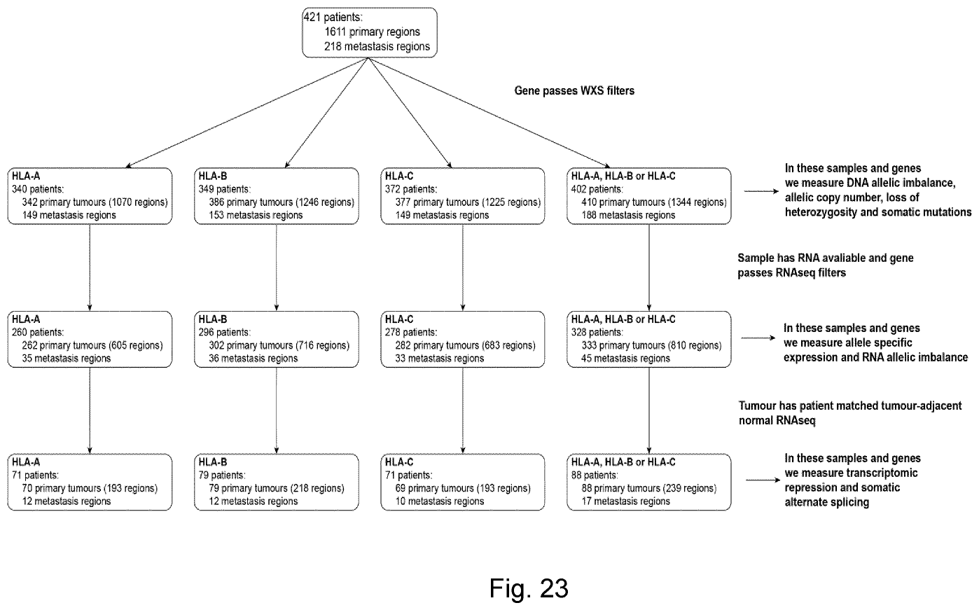

These examples describe and demonstrate the use of MHC Hammer (MHC loss of Heterozygosity, Allelle-specific Mutation, and Measurement of Expression and Repression), a computational toolkit to accurately determine allele-specific mutations, LOH, allelic expression, allelic repression, and alternative splicing of the class I HLA genes. The examples further demonstrate how MHC Hammer can be used to investigate the role of HLA genomic and transcriptomic disruption in tumour evolution using a multiregional cohort of prospectively recruited 421 NSCLC patients in TRACERx. HLA LOH and mutations were measured in 1344 primary tumour regions and 188 metastasis regions from 402 patients. The method enabled to quantify HLA repression and alternative splicing in 239 primary tumour regions and 17 metastasis regions from 88 patients with a patient-matched tumour-adjacent normal sample (FIG. 23).

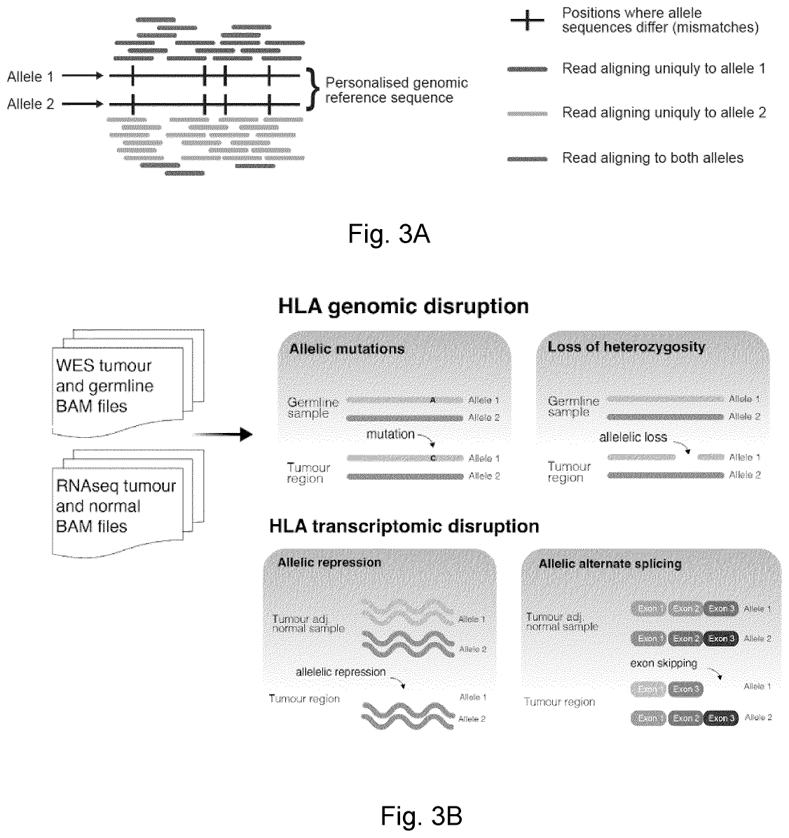

The pipeline has five components (see FIG. 3B): 1) determining the patient's HLA allele type and creating patient-specific reference files; 2) calculating HLA allele-specific copy number and LOH; 3) determining the presence of allele-specific HLA somatic mutations; 4) evaluating HLA allele-specific expression and repression; 5) evaluating the presence of allele-specific HLA alternative splicing.

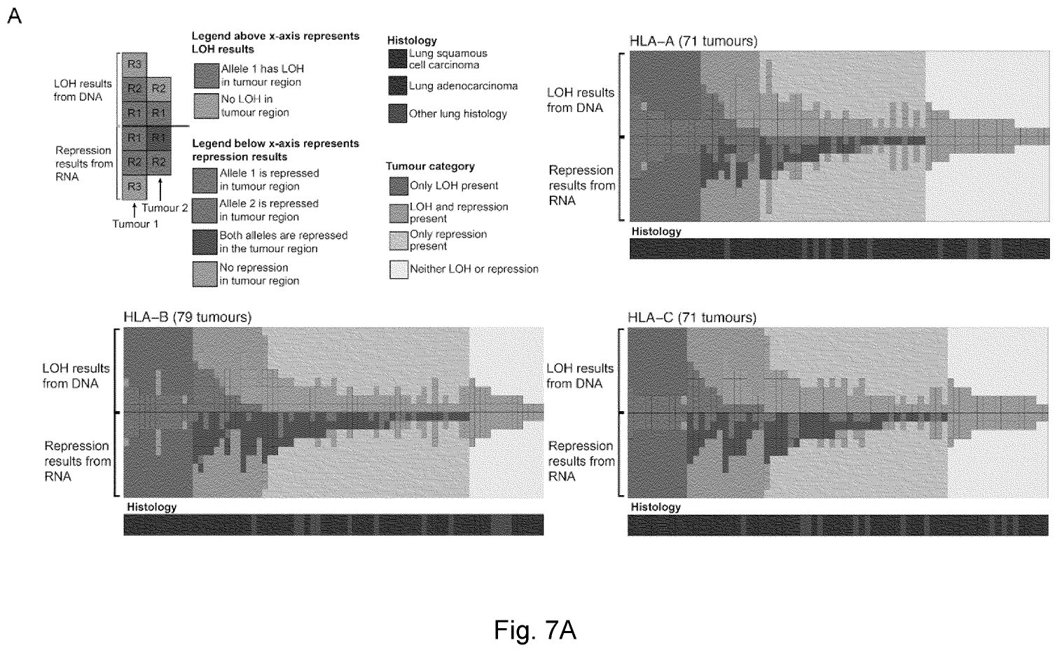

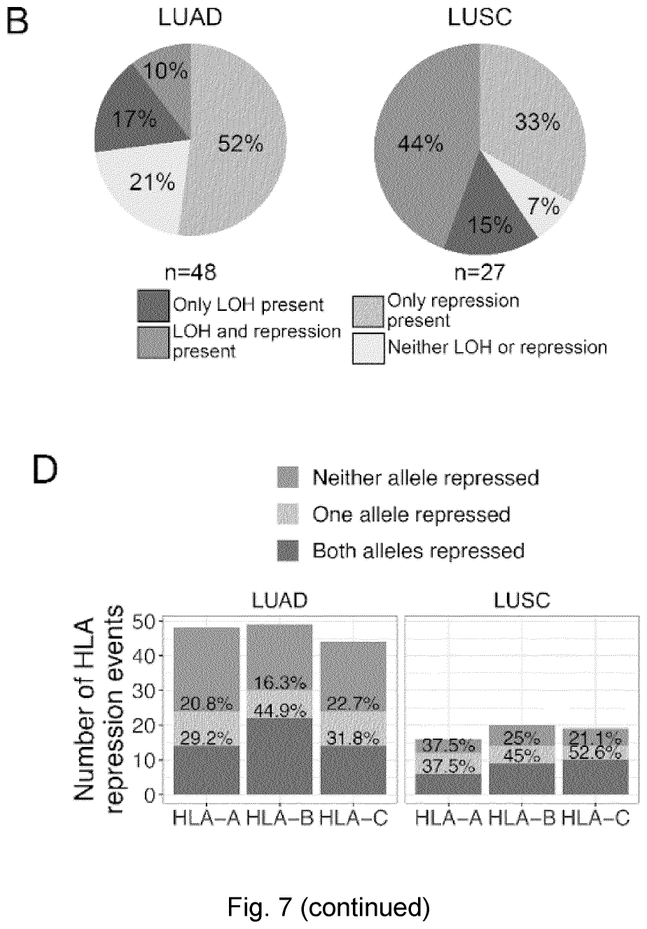

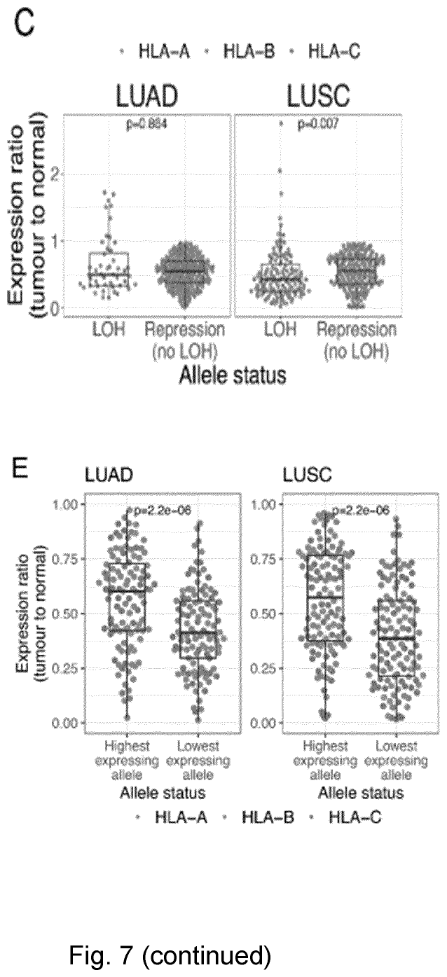

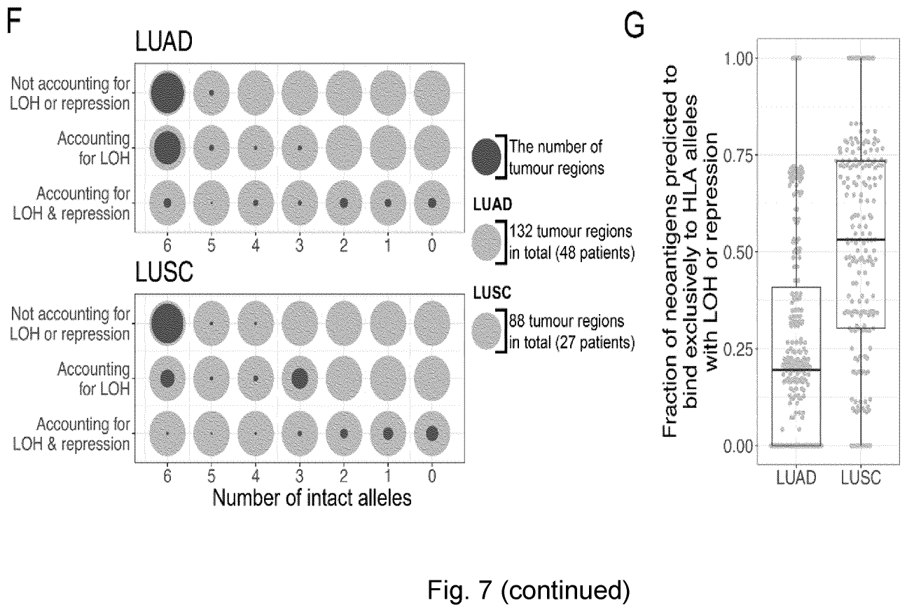

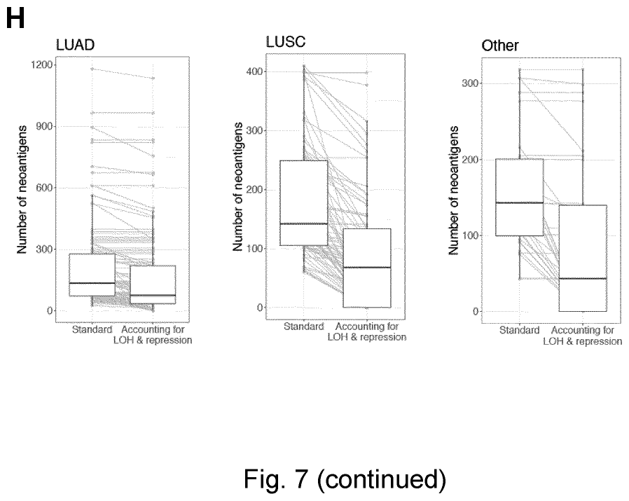

The method takes as input RNA and DNA sequence data (e.g. WES and bulk RNAseq data, for example in the form of BAM files) (DNA sequence data from tumour and matched normal/germline, RNA sequence data from tumour and optionally from matched normal if available). Parts of the method further use as input aberrant cell fraction (purity) and tumour ploidy predictions for each tumour sample. The method subsequently predicts the fractional allele specific copy number and the expression of each HLA allele (by default for class I alleles but versions including predictions for both class I and class II alleles are also demonstrated) within individual tumour samples. This is explained in detail in Example 1. This work demonstrates that to fully capture the extent of HLA disruption in cancer it is imperative to consider both the DNA and RNA (FIG. 7).

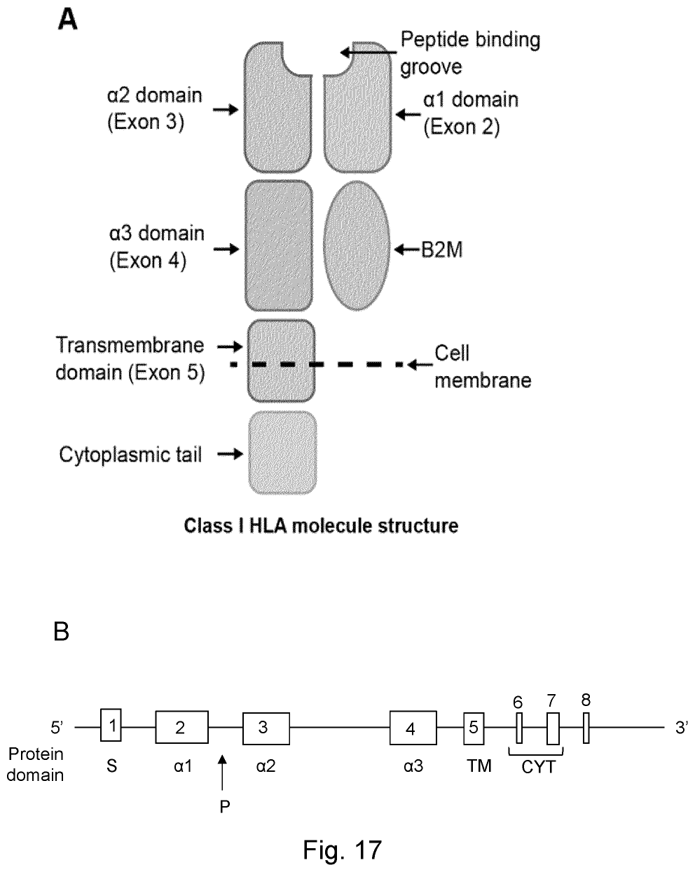

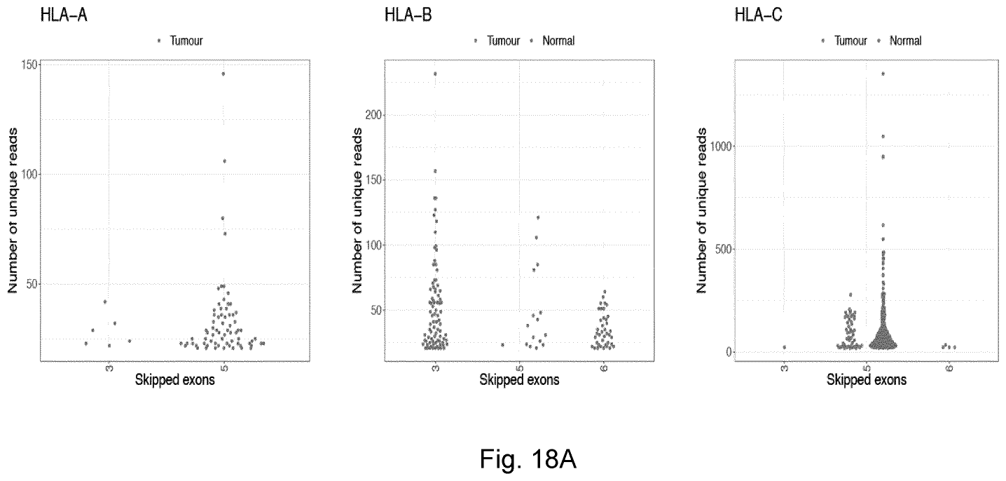

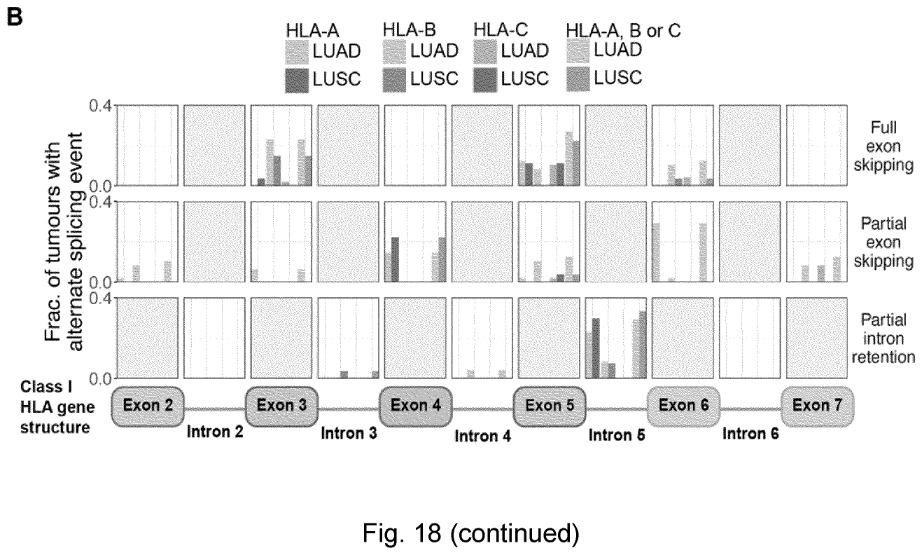

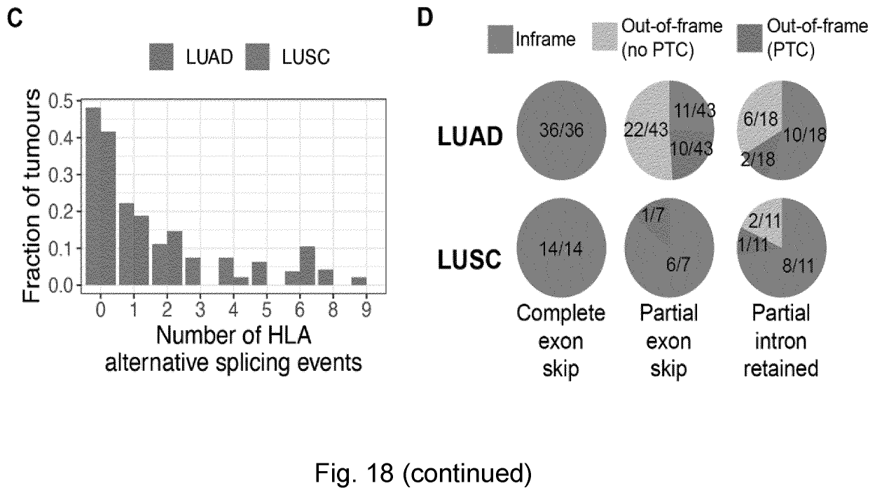

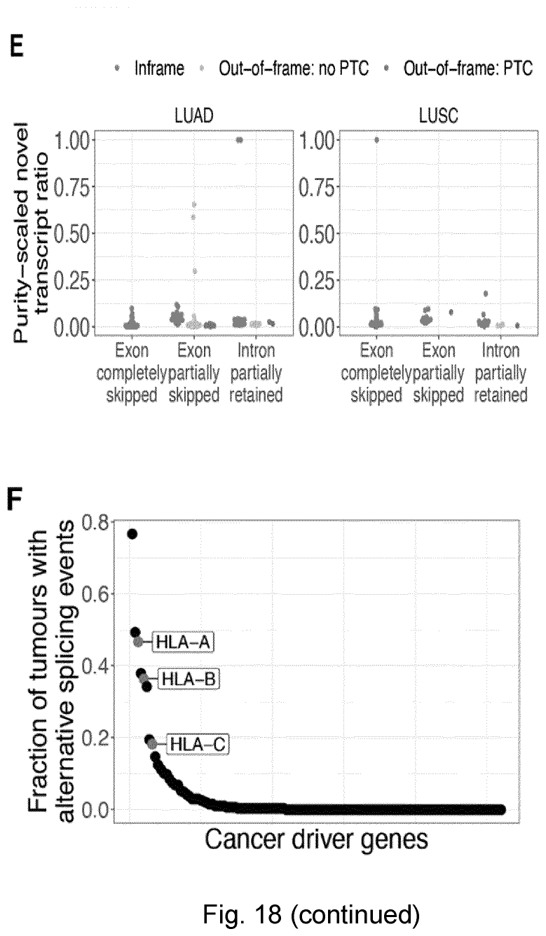

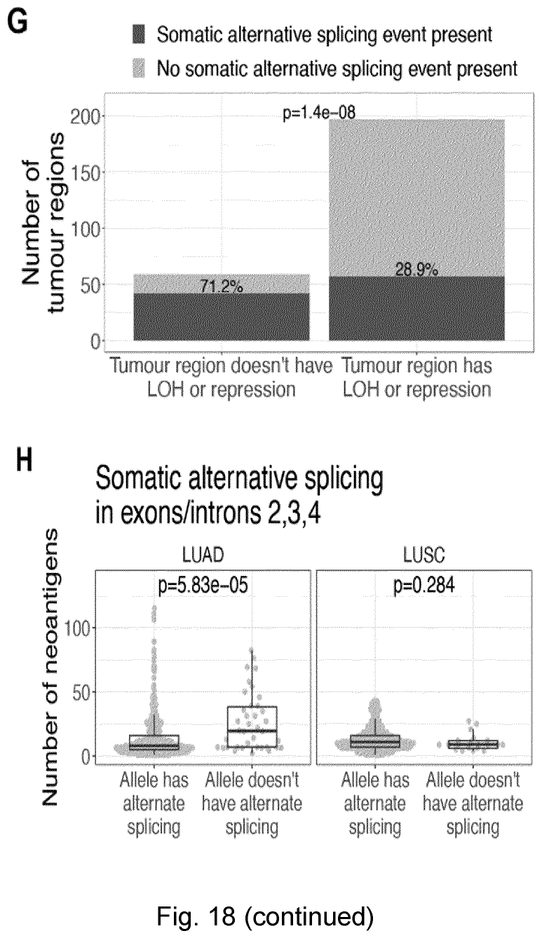

The classical HLA class I gene is organized into eight exons with distinct functional domains (FIG. 17B). Exon 1 encodes the signal peptide, while exons 2, 3 and 4 encode the α1, α2 and α3 domains respectively. Exon 5 encodes the transmembrane domain and the cytoplasmic tail is encoded by the remaining three exons. Thus, alternative splicing of a given exon may yield an altered yet functional protein, conceivably disrupting HLA presentation and limiting tumour immunogenicity. The inventors developed a method to evaluate splicing deregulation (such as e.g. exon skipping), by identifying high-quality split sequencing reads. This work demonstrates that HLA alternative splicing can be identified in both tumour and tumour-adjacent normal tissue (FIG. 18). The same principle can be applied to HLA class II genes. HLA class II molecules are heterodimers comprising an α and β chain, both of which are encoded in the MHC. The chains comprise extracellular domains α1, α2, and β1, β2, respectively, as well as a transmembrane region and a cytoplasmic tail. The α1 and β1 regions of the chains together form the peptide-binding domain. In the beta chain, exon 1 encodes the signal peptide, exons 2 and 3 encode the two extracellular domains, exon 4 encodes the transmembrane domain and exon 5 encodes the cytoplasmic tail. In the alpha chain, exon 1 encodes the signal peptide, exons 2 and 3 encode the two extracellular domains, and exon 4 encodes the transmembrane domain and the cytoplasmic tail. Thus, the loss of the respective exons encoding the transmembrane domain or peptide binding domains in the HLA class II genes may have similar consequences to the loss of corresponding domains in class I genes.

Thus in these examples the inventors present a method permitting detailed genomic and transcriptomic evaluation of allele specific HLA disruption (including allele specific expression, alternative splicing including exon skipping—as well as the previously demonstrated mutation calls and LOH calls—for the class I and class II HLA genes), and demonstrate its use in evaluating the extent of genomic and transcriptomic HLA disruption in cancer using data from the TRACERx project.

### Methods

### HLA Deregulation Pipeline

The examples below demonstrate the use of a pipeline referred to as “MHCHammer”. The pipeline involves the following steps.

Constructing complete HLA allele sequences. The HLA allele sequence information stored within the publicly available ImMunoGeneTics (IMGT, Lefranc 2003) database is used to construct the HLA references used by MHC Hammer. However, not all HLA alleles have a complete sequence available. If an allele is missing sequence information, the coding sequence that is available for that allele is used to determine which other allele with a complete sequence is the most similar. The missing sequence is then completed using the sequence of the similar allele. This is a similar method to an approach used in a previous study (Shukla et al., 2015).

Subsetting the input BAM files to only include potential HLA reads. To reduce the computational resources required by MHC Hammer, the input WES and RNAseq BAM files are filtered to keep only ‘potential’ HLA reads. This includes reads that: (i) Map to chromosome 6 in the input BAM file; (ii) Map to any contig in the input BAM file; (iii) Are unmapped in the input BAM file; or (iv) Contain a 30-base pair sequence that exists in the constructed HLA reference. Contigs are additional “pseudo-chromosomes” that may be included in standard reference sequences. For example, in the hg19 reference, there are 6 contigs that contain the sequence of 6 different HLA haplotypes.

Predicting the patient's germline HLA alleles. As the MHC region is one of the most polymorphic regions of the genome, accurate quantification of genomic and transcriptomic events requires a patient-specific approach. Therefore, the MHC Hammer pipeline first determines the patient's HLA allele type from the patient's germline whole exome sequencing (WES) data using HLA-HD, an accurate HLA typing algorithm designed for next-generation sequencing data (Kawaguchi et al., 2017; Liu et al., 2021).

Constructing the patient-specific HLA references. For each patient, a genomic reference, a transcriptomic reference and a gene transfer format (GTF) file is created using the HLA-HD allele predictions and the complete allele sequence information. GTF files contain information about gene structure, in this case at least the locations of exons, introns and UTRs (untranslated regions).

Constructing the WES HLA allele BAM files. To construct the WES HLA allele BAM files, the potential HLA reads are mapped to the patient's genomic reference using NovoAlign (http://novocraft.com/) in a way that allows reads to map to multiple alleles. Similar to the approach in McGranahan et al., 2017, these BAM files are then filtered such that reads whose mates map to a different allele are discarded, as well as any read that contains more than one insertion, deletion, or mismatch event compared to the reference HLA allele. The WES HLA allele BAM files are used to calculate allelic copy number and DNA allelic imbalance, which in turn are used to infer HLA LOH.

Creating the RNAseq allele BAM files. Two versions of RNAseq HLA BAM files are created in the MHC Hammer pipeline. The first version is created by mapping the potential HLA RNAseq reads to the patient's transcriptomic reference using Novoalign (novocraft.com/) in a way that allows reads to map to multiple alleles. Similar to the approach in McGranahan et al. 2017, these BAM files are then filtered such that reads whose mates map to a different allele are discarded, as well as any read that contains more than one insertion, deletion or mismatch event compared to the reference HLA allele. These BAM files are used to detect RNA allelic imbalance, allelic expression and allelic repression estimates. The second version is created by mapping the potential HLA RNAseq reads to the patient's genomic reference using the STAR aligner (Dobin et al., 2013) and the patient's GTF file. To reduce the computational time used by STAR, the potential HLA RNAseq reads are further filtered to only those that contain a 30-base pair sequence from the constructed HLA genomic reference. STAR can account for gaps in the alignment resulting from introns or alternative splicing. A two pass alignment is used to improve accuracy (Veeneman et al., 2016). Specifically, all samples are aligned once with STAR; then, the splice junctions detected across the cohort in the first alignment with at least 3 supporting reads are used as input to the second STAR alignment. The final splice junction table from the second STAR alignment is used to detect HLA alternative splicing. The second BAM (or any BAM obtained by aligning the RNAseq reads to the patient's genomic reference) may also be used to detect RNA allelic imbalance, allelic expression and/or allelic repression estimates.

Getting the gene SNP positions. For a given HLA gene, the single nucleotide polymorphism (SNP) positions are the base pair positions where the sequence of the two alleles differ. This is calculated using the Needleman-Wunsch algorithm implemented in the R package pairwise Alignment from the Biostrings library. These SNP positions are filtered for those that pass the minimum coverage of 30× in both alleles of the gene in the germline WES sample.

Calling HLA loss of heterozygosity. Similar to the method in McGranahan et al. 2017, for a given class I HLA gene, HLA LOH is called if: (1) The copy number of the minor allele is less than 0.5; and (2) There is DNA allelic imbalance (AIB). The coverage at the filtered SNP positions is used to calculate the B-Allele Frequency (BAF) and log R, which are then used to calculate the copy number of the alleles. For each filtered SNP i, the BAF is calculated as:

$${BAF}_{i} = \frac{allele 1 tumour depth at SNP i}{total depth}$$

where the choice of allele 1 is arbitrary. At each filtered position in the allele, the Log R is calculated as the tumour depth at that position divided by the germline depth at that position and normalised for differences in library size by multiplying the germline library size divided by the tumour library size. The library size is the number of unique reads in the input tumour and germline BAM files and is calculated using samtools flagstat (Li et al., 2009). The median log R is calculated for each 150-base pair bin across the gene. The copy number of each filtered SNP in the two alleles is then calculated as

$$allele 1 {CN}_{i} = ρ - 1 + {BAF}_{i} \times {2}^{{logR}_{i}} \times \frac{( 1 - ρ ) \times 2 + ρφ}{ρ}$$

$$allele 2 {CN}_{i} = ρ - 1 - ( {BAF}_{i} - 1 ) \times {2}^{{logR}_{i}} \times \frac{( 1 - ρ ) \times 2 + ρφ}{ρ}$$

where ρ is the purity of the tumour region, φ the ploidy of the tumour region and log Rᵢ is the log R of the bin that SNPᵢ falls in. The allele copy number is the median copy number across the SNPs.

To estimate DNA AIB, the coverage at the SNP positions is adjusted so that each sequencing read is only counted once per SNP (Castel et al., 2015). A Wilcoxon test with a p-value cutoff of 0.01 is then used to determine if there is a significant difference in the log R at the SNP positions between the two alleles. The Wilcoxon test is calculated using the R wilcox.test function from the stats library.

To increase the accuracy, three additional filters were added compared to the original LOHHLA method described in McGranahan et al., 2017. The first filter is based on the expected depth of the allele in the tumour region. For a given allele, the expected depth of a SNP is the depth of the SNP in the germline allele BAM file multiplied by the purity of the tumour region and the tumour region library size divided by the germline region library size. The expected depth of the allele is the median of the expected depth at the filtered SNP positions. In this study, we excluded alleles that had an expected depth of less than 10 from further analysis, as in these cases we would not expect to have the required coverage to accurately classify LOH even if it were present. The second filter is based on the width of the confidence intervals of the allelic copy number estimate. A 95% confidence interval in the allelic copy number is calculated and samples are excluded from further analysis if the interval spans more than 2.5 copies, as this indicates particularly noisy data, which could lead to inaccurate LOH calls. The third filter is based on the number of filtered SNPs. If a gene had less than 3 filtered SNPs, the gene was excluded from further analysis.

Calling HLA mutations. Mutect2 is used to call HLA allele-specific somatic mutations in the filtered WES HLA allele BAM files, following GATK best practice guidelines (Benjamin et al., 2019). The Ensembl Variant Effect Predictor (VEP) is used with the sequence information in the IMGT database to determine the consequence of the somatic mutations (McLaren et al., 2016). Somatic mutations called in alleles that are also predicted to be subject to deletion, resulting in LOH, are likely to be false positives. Reassuringly, we only observed 67/411, 30/324, and 26/302 mutations on clonally lost alleles. In most cases where we observed paradoxical mutation and loss, we found significantly less support for the mutated allele (alternate read <10). We therefore additionally filtered our mutation calls to only include those with an alternate depth greater than 10.

Estimating HLA gene and allelic expression. To quantify HLA allelic expression, an updated read count for each allele that takes into account reads that map to both alleles of the given gene is first calculated. To do this, the fraction of reads that map only to allele 1 (f₁) is defined as:

$${f}_{1} = \frac{{r}_{1}}{{r}_{1} + {r}_{2}}$$

where r₁ is the number of reads that map uniquely to allele 1 and r₂ is the number of reads that map uniquely to allele 2. Using f₁, an updated read count for each allele is calculated as:

$${R}_{1} = {r}_{1} + {f}_{1} \times {r}_{12}$$

$${R}_{2} = {r}_{2} + ( 1 - {f}_{1} ) \times {r}_{12}$$

where r₁₂ is the number of reads mapping to both alleles of a given gene, R₁ is the updated read count for allele 1, and R₂ is the updated read count for allele 2. Using this updated read count, the allele RPKM (reads per kilobase million) is calculated for allele 1 (RPKM₁) and allele 2 (RPKM₂) as:

$${RPKM}_{1} = \frac{{R}_{1} / l}{{a}_{1}}$$

$${RPKM}_{2} = \frac{{R}_{2} / l}{{a}_{2}}$$

where l is the number of unique reads in the original RNAseq BAM file, calculated using samtools flagstats (Li et al, 2009), a₁ is the length of allele 1 and a₂ is the length of allele 2. The gene level RPKM is calculated as:

$$RPKM = \frac{( {r}_{1} + {r}_{2} + {r}_{12} ) / l}{( {a}_{1} + {a}_{2} ) / 2}$$

To increase accuracy, MHC Hammer implemented two filters based on the RNAseq data. Firstly, at least 50% of reads mapping to an HLA gene must map uniquely to an allele. Secondly, a gene must have 3 SNPs that pass the minimum coverage in both alleles in the transcriptome HLA allele BAM files.

Calling HLA RNA A/B. To estimate RNA AIB, the coverage at the SNP positions is adjusted so that each sequencing read is only counted once per SNP (Castel et al., 2015). A Wilcoxon test with a p-value cutoff of 0.01 is then used to determine if there is a significant difference in the coverage at the SNP positions between the two alleles. The Wilcoxon test is calculated using the R wilcox.test function from the stats library.

Calling HLA transcriptomic repression. HLA transcriptomic repression is only defined in tumours with a patient-matched tumour-adjacent normal sample. An allele is defined as repressed in a tumour region if it has a significantly lower read depth in the tumour compared to the tumour-adjacent normal sample at the SNP positions, using a Wilcox test with a p-value cutoff of 0.01. The tumour and normal depths are normalised for differences in library size.

Calling full intron retention. To detect full intron retention, the coverage of the exons and introns is calculated using mosdepth (Pedersen & Quinlan, 2018). We did not see any significant coverage across the introns of the HLA alleles.

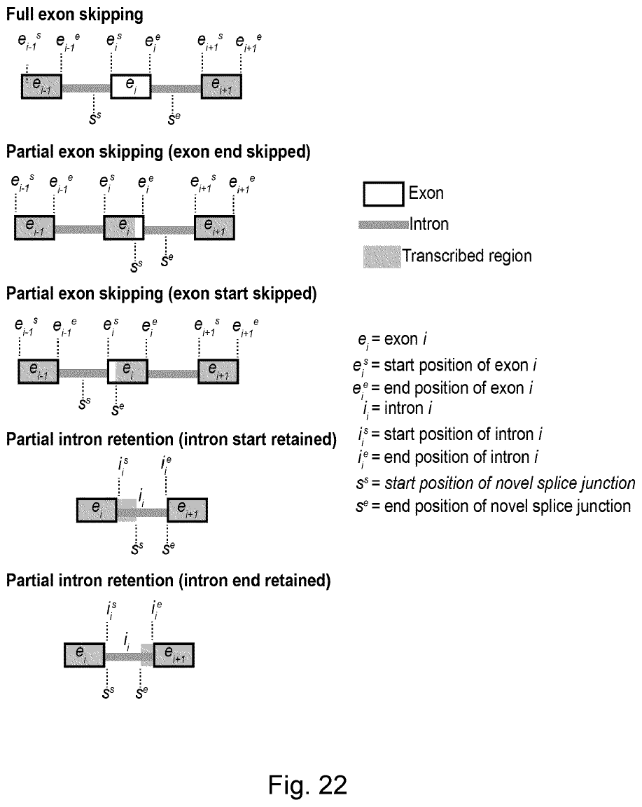

Calling full exon skipping, partial exon skipping, and partial intron retention. The second STAR alignment outputs a table of splice junctions detected in each sample. Using the GTF file, MHC Hammer categorises these splice junctions as either known (if they are present in the GTF file) or novel. Each novel splice junction is then classified as either a full exon skipping event, a partial exon skipping event or a partial intron retention event using the following definitions (illustrated on FIG. 22), where S^{s}=the start position of the novel splice junction, S^{e}=the end position of the novel splice junction, eᵢ=exon l, eᵢ^{s}=start position of exon l, eᵢ^{e}=end position of exon l, iᵢ=intron l, iᵢ^{s}=the start position of intron l, and iᵢ^{e}=the end position of intron i:
A novel splice junction is defined as a full exon skipping event of exon eᵢ if eᵢ₋₁^{e}<S^{s}<eᵢ^{s} and eᵢ^{e}<S^{e}<eᵢ₊₁^{s}
A novel splice junction is defined as skipping the end of an exon eᵢ (partial exon skipping) if eᵢ^{s}<S^{s}<eᵢ^{e}
A novel splice junction is defined as skipping the start of an exon eᵢ (partial exon skipping) if eᵢ^{s}<S^{e}<eᵢ^{e}
A novel splice junction is defined as retaining the start of an intron iᵢ (partial intron retention) if iᵢ^{s}<S^{s}<iᵢ^{e}
A novel splice junction is defined as retaining the end of an intron iᵢ (partial intron retention) if iᵢ^{s}<S^{e}<iᵢ^{e}.

Each novel spice junction is also filtered based on the number of uniquely mapping reads supporting the corresponding known splice junction. The read count of the corresponding known splice junction is defined as:
Full exon skipping: The average number of uniquely mapping reads supporting the splice junction joining exons eᵢ₋₁ and eᵢ and the splice junction joining exons eᵢ and eᵢ₊₁
Exon end skipping: the number of uniquely mapping reads supporting the splice junction joining exons eᵢ and eᵢ₊₁
Exon start skipping: the number of uniquely mapping reads supporting the splice junction joining exons eᵢ₋₁ and eᵢ
Intron start retained: the number of uniquely mapping reads supporting the splice junction joining exons eᵢ and eᵢ₊₁
Intron end retained: the number of uniquely mapping reads containing the the splice junction joining exons eᵢ and eᵢ₊₁

A novel splice junction is filtered and removed from further analysis if it is supported by less than 50 reads.

Determining the consequence of the alternative splicing events. To determine the consequence of an alternative splicing event, the new sequence that results from the novel splice junction is calculated. For full or partial exon skipping, the new sequence is calculated by removing the coding sequence that falls between the start and end of the novel splice junction. For events where the start of an intron is retained, the new sequence is calculated by inserting the sequence from the start of the intron to the start of the novel splice site. For events where the end of an intron is retained, the new sequence is calculated by inserting the sequence from the end of the novel splice junction to the end of the intron. Once the new sequence has been calculated, the alternative splicing event is defined as inframe if the number of bases in the new sequence is divisible by 3. The alternative splicing event is defined as introducing a premature termination codon (PTC) if a PTC exists in the new sequence.

Purity-scaled ratio of novel-to-canonical transcripts for alternative splicing events. To estimate the fraction of cancer cells that harboured a somatic alternative splicing event, a purity scaled ratio of novel-to-canonical transcripts is calculated. To do this, the ratio of novel-to-canonical transcripts is defined as the number of uniquely mapping reads containing the novel splice junction divided by the number of uniquely mapping reads containing the corresponding known splice junction, as defined above. This ratio is then divided by the purity to account for the non-cancer cells in the tumour region. As the purity-scaled ratio represents the fraction of cancer cells that carry the somatic alternative splicing event, the purity-scaled ratio is capped at 1.

### Validation of Allele-Specific HLA Alternative Splicing

To validate our HLA alternative splicing pipeline, we used allele-specific polymerase chain reaction (PCR) amplification. The fragment sizes were confirmed via agarose gel electrophoresis. These PCR products were then cloned using a TA cloning kit (Invitrogen), where the wild-type and novel alleles were subsequently validated through Sanger sequencing. We performed this for 2 tumour regions and one normal sample from the same patient (CRUK0061). MHC Hammer identified exon 5 skipping in the HLA-C*16 allele in both tumour regions and the tumour-adjacent normal sample.

To amplify each allele, we used the allele-specific primers that have been described in Gerritsen (2016), 3′ primer is HLA-C primer in Table2A, 5′ primer is C*16 5′ primer in Table 2B of Gerritsen (2016). We were able to identify both the novel and canonical transcripts of the alternative splicing event (see Table 1 below).

TABLE 1
Results of HLA allele-specific alternative splicing validation by PCR.

| | | Ratio of novel-to-known | |
|---|---|---|---|
| | | transcripts from MHC | Alternative splicing |
| Sample name | Sample type | Hammer | detected using PCR |
| CRUK0061_SU_T1-R1 | Tumour region | 0.12 | Yes |
| CRUK0061_SU_T1-R2 | Tumour region | 0.08 | Yes |
| CRUK0061_SU_N01 | Tumour-adjacent | 0.1 | Yes |
| | normal sample | | |

TRACERx copy number data. Purity and ploidy estimates used in this study were taken from a previous TRACERx study (Frankell et al., 2023).

WES sample collection and sequencing. Details can be found in Frankell et al., in press.

RNAseq sample collection and sequencing. Details can be found in Martinez-Ruiz et al., 2023.

Neoantigen calls. Patient-specific HLA haplotype predictions were obtained using HLA-HD (Kawaguchi et al. 2017) (version 1.2.1). NetMCHpan4.1 (Reynisson et al. 2020) was run on 9-11 neopeptides derived from nonsynonymous mutations across the TRACERx421 cohort and taking into account patient-specific HLAs. A cut-off of 0.5 in the Eluted Ligand rank was applied to define whether a peptide bound to a specific HLA type. An observed nonsynonymous mutation is deemed a neoantigen binding to a specific HLA if at least one of its neopeptides is considered a binder.

CIBERSORTx. CIBERSORTx (Newman et al., 2019) was run on the TPMs of the entire Tx421 cohort using the LM22 signature.

Statistical information. All statistical tests were performed in R. No statistical methods were used to predetermine sample size. Tests involving comparisons of distributions were done using a two-tailed Wilcoxon test (‘wilcox.test’). Tests involving comparison of groups were done using two-tailed Fisher's exact test (‘fisher.test’). Hazard ratios and P values were calculated with the ‘survival’ package. Correlation was tested using the Pearson's correlation coefficient (‘cor.test’).

### Example 1—Allele Specific Transcriptional Repression of the Class I HLA Genes in the TRACERx Non-Small Cell Lung Cancer Tumours

The HLA class I genes, HLA-A, HLA-B and HLA-C, encode the class I cell surface proteins that present endogenous peptide fragments (antigens) to CD8 T cells. Both alleles of each gene are expressed and can form cell surface proteins. In cancer cells the class I proteins can present somatic mutations (neoantigens). If these neoantigens are recognised by CD8 T cells this can lead to an anti-tumour immune response. Previous work from the inventors has shown that 40% of the first TRACER×100 primary tumours had loss of heterozygosity (LOH) of at least one of the HLA genes {McGranahan et al., 2017}. LOH refers to a single allele of a gene being lost in the DNA. Using TRACERx421 RNAseq data, the present inventors set out to determine whether there was additional repression of the HLA alleles at the transcript level. The TRACERx421 RNAseq data consists of 941 primary tumour regions from 357 patients. Of these patients 91 also have RNAseq from matched tumour adjacent normal samples.

### Quantifying HLA Class I Allelic Expression and Allelic Imbalance

The HLA region is one of the most polymorphic regions of the genome. This means the sequence of a patient's HLA alleles can be very different to the standard reference genome. This can result in inaccurate copy number calls and expression estimates for the HLA genes. It is also impossible to get allele specific copy number and expression using the standard reference genome. To overcome this, we created a personalised reference for each patient, using the sequence of the patient's alleles, which are predicted from the matched whole exome sequencing data using HLA-HD {Kawaguchi et al., 2017}. It is believed that HLA typing from genomic data is more suitable for the purpose of the present method than HLA typing from RNA (expression) data because the latter typically only provides two digits allele resolution (i.e. allele group only). It is advantageous for the purpose of the present methods that HLA typing is performed to full resolution (i.e. including the allele group and specific HLA protein). This uses all human alleles for class I, I, Ib and IIb stored in the IPD-IMGT/HLA database. Reads are then mapped to the personalised reference. In particular, potential HLA reads from an input BAM file (from RNAseq—obtained as described in Jamal-Hanjani et al., 2017) were extracted and mapped to the patient specific reference (provided as a genomic file in FASTA format comprising the sequence of the identified alleles and a GTF file specifying the locations of introns and exons) using STAR {Dobin et al., 2013}. Alternative tools may be used such as e.g. NovoAlign (Novocraft Technologies). In this work, NovoAlign was used when quantifying LOH by mapping to a genomic reference. Reads were defined as potential HLA reads if they were aligned to chromosome 6 or were unmapped in the BAM file or if they contained (exact match) any string from a list of k-mers obtained from reference sequences of the HLA locus (where k was chosen as 30, and reference sequences of the HLA locus were chosen as all the sequences available in the IPD-IMGT/HLA database). Alternatively, reads that were aligned to chromosome 6 or were unmapped in the BAM file may be used.

To determine if there is imbalance in the expression of the two alleles (allelic imbalance), a paired Wilcoxon test of the depth at the positions where the allele sequences differ (mismatches) is used (see FIG. 3A).

To quantify allelic expression, we normalise the number of reads that map uniquely to each allele for total coverage and allele length. Our unit of expression is transcripts per thousand (TPT). In particular, we divide the allele read count by the length of each allele in kilobases, giving reads per kilobase (RPK). We then calculate a scaling factor for each sample, by summing the RPK values for the class I and II HLA genes and dividing by a factor of 1000 (simply for ease of reading of resulting metrics—this may not be used or may be set to a different value). Our metric of expression, transcripts per thousand (TPT), is then calculated as the RPK value divided by the scaling factor.

Because the measure of HLA expression defined above is dependent on allele read count, accurate estimation can be affected by the presence of multi-mapping reads. For each sample and gene, we calculated the fraction of reads that map: uniquely to a single allele; to both alleles in a single gene; and to multiple genes. We found that the mean number of reads that mapped to multiple genes per sample was 3.6% for class I genes. There were 12 samples that had more than 10% of reads mapping to multiple genes in HLA-A and 4 samples with more than 10% of reads mapping to multiple genes in HLA-C. To ensure that we did not overestimate HLA expression due to reads mapping to multiple genes, we excluded genes that and samples that had more than 10% of reads aping to multiple genes. This excluded HLA-A estimates from 12 samples, and HLA-C estimates from 4 samples.

The mean number of reads that map to both alleles from the same gene was 7.0% for class 1. Reads that map to both alleles are added to the unique allelic read count using the ratio of uniquely mapped reads (in other words, we redistributed the multi mapping reads to the allele read count, by multiplying the number of multi mapping reads by the allele ratio of uniquely mapping reads for that gene). Alternatively, reads that map to both alleles may be redistributed to the allele read counts using a maximum likelihood approach such as that described in e.g. Aguiar et al., 2020. To call allelic expression and imbalance, we require at least 50% of the reads to map uniquely (i.e. to a single allele). Thus, for these samples a gene level analysis (rather than allele level analysis) was performed.

### HLA Allele Expression and Imbalance in Tumour Adjacent Normal Samples

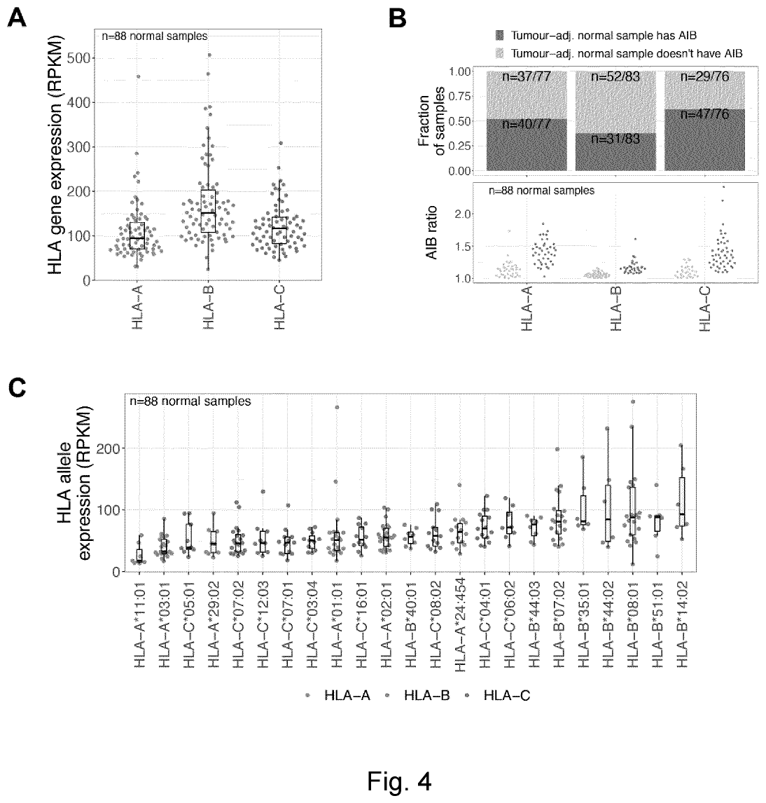

HLA expression may be both gene- and allele-specific, even without underlying somatic copy number alterations. Therefore, to understand HLA allele-specific expression in the absence of copy number alterations, we first quantified HLA allele expression in the tumour adjacent normal samples. When restricting our analysis to each of the class I HLA genes, we found that HLA-B had the highest median expression, followed by HLA-C, then HLA-A (HLA-A: 94.2 RPKM, HLA-B: 151.8 RPKM, HLA-C: 116.8 RPKM). We found a wide range in HLA expression across the three genes (HLA-A: 30.5-458.3 RPKM, HLA-B: 24.9-506.8 RPKM, HLA-C: 45.8-308.9 RPKM, FIG. 4A). Utilising the allele-specific output from MHC Hammer, we then evaluated differential expression between alleles for each sample and gene, using an allelic imbalance (AIB) ratio. For each gene, this was calculated as the expression of the allele with the higher expression divided by the expression of the allele with lower expression. The mean AIB ratio was 1.4 for HLA-A, 1.2 for HLA-B, and 1.4 for HLA-C (range: 1.0-1.8 (HLA-A), 1.1-1.6 (HLA-B), 1.1-2.4 (HLA-C), FIG. 4B). Strikingly, 51.9% of tumour-adjacent normal samples exhibited statistically significant allelic imbalance expression in HLA-A, 37.3% in HLA-B, and 61.8% in HLA-C. Thus, even without copy number alterations, HLA is subject to widespread expression imbalance. The data revealed evidence of a relationship between allelic expression and the allele type (FIG. 4C). HLA-A*24:454, HLA-B*14:02, and HLA-C*06:02 had the highest expression across the three genes, while HLA-A*11:01, HLA-B*40:01, and HLA-C*05:01 had the lowest. Taken together, these data suggest that HLA allele-specific expression imbalance is frequent in normal tissue and that total HLA gene expression is strongly influenced by the combination of HLA alleles that a person harbours. These data emphasise the importance of controlling for HLA allelic expression in normal tissue when assessing transcriptional alterations in tumour-derived HLA allelic signals.

In other words, this data demonstrates that quantification of HLA allele expression deregulation in tumours must take into account a comparable normal level of expression in order to be meaningful.

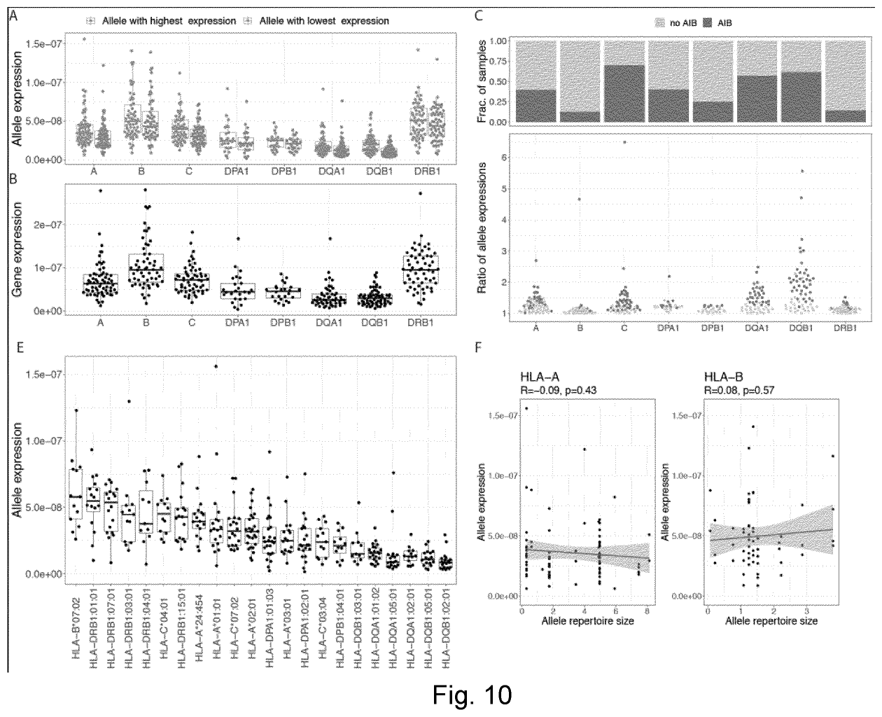

We also found a wide range (18.2 TPT-571.5 TPT) in the total gene level expression in the tumour adjacent normal samples, with HLA-B having the highest expression in the class I genes and HLA-DRB1 having the highest expression in the class II genes (FIG. 10A). In general, the amount of expression per allele followed the same pattern as the gene level expression, i.e. both alleles of HLA-B had the highest expression in the class I alleles, and both alleles of HLA-DRB1 had the highest expression in the class II alleles (FIG. 10B). For each sample and gene, we then looked at how much more expression was coming from one allele compared to the other. On average, when there was significant allelic imbalance in a gene, the allele with the higher expression had twice the expression than the allele with the least expression (FIG. 10C-D). To investigate whether we saw a relationship between expression and HLA lineage in the normal samples, we plotted the allele expression as a function of the HLA allele type. We found that there was a relationship between the allelic expression and the allele type (FIG. 10E).

Comparison of the Method with Existing Approaches

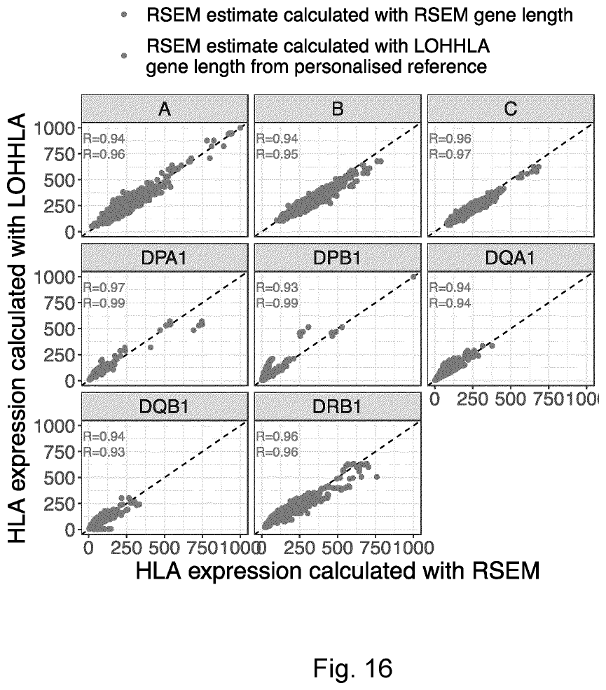

We benchmarked our estimation of gene level HLA expression with the gene expression predicted by RSEM (which does not use a patient specific reference and simply aligns to a common standard genome reference). To do this, we recalculated our TPT metric using the expected read count and read length output by RSEM (replacing the allele read count in the TPT metric described above by the expected counts for the gene as provided by RSEM), and found good concordance (FIG. 16), indicating that our approach does not produce completely spurious results. However, in HLA-B and HLA-C, RSEM overestimated the expression compared to LOHHLA, and in HLA-DPA1, HLA-DPB1, RSEM underestimated the expression compared to LOHHLA. To understand what was driving the differences between LOHHLA and RSEM, we considered the two factors that would likely impact the estimation of expression: the gene length and the gene read count. We found that the average length of the two HLA alleles for each gene in the IMGT database differed from what was used by RSEM, and if we used the average allele length instead of RSEM's effected length when calculating RSEMs expression estimates there was much better concordance (FIG. 16, compare blue series and red series).

### Measuring HLA Allele Expression in the TRACERx421 Tumour Regions

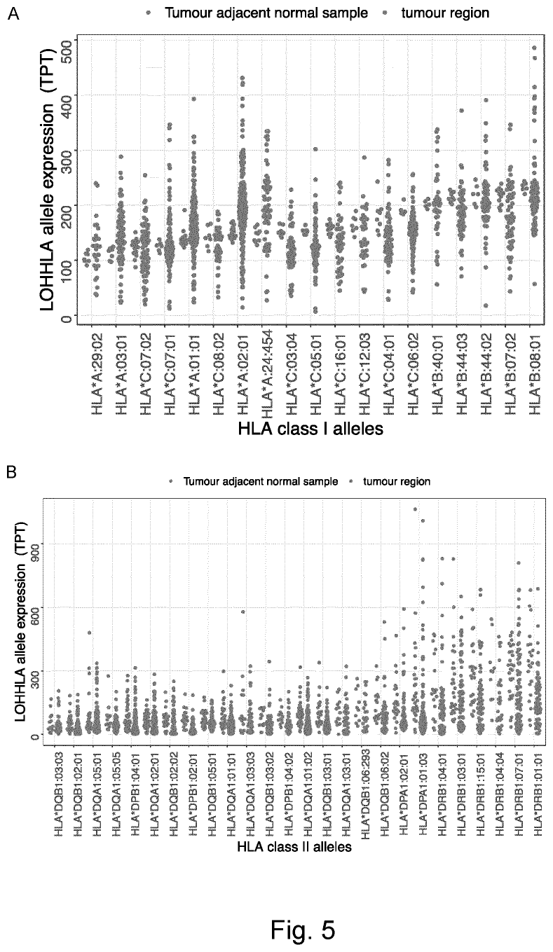

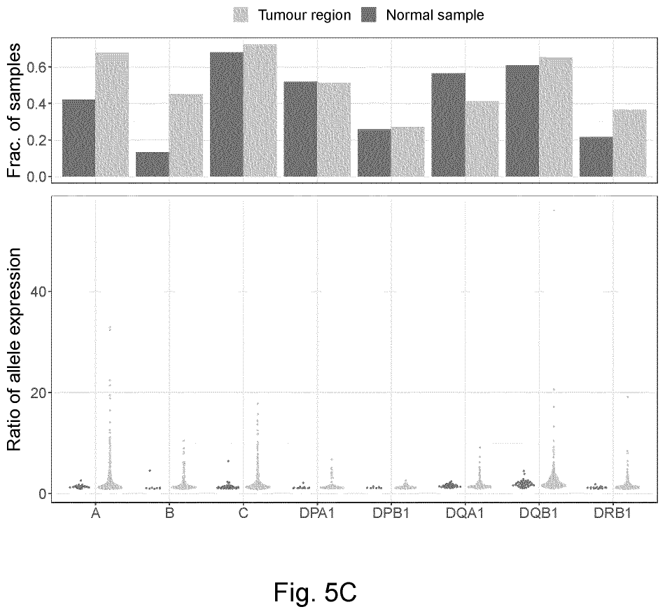

Looking at the tumour regions alone, we found that there was a wider range in allele expression in the tumour regions compared to the tumour adjacent normal samples (FIG. 5A-B). The tumour regions also had higher rates of allelic imbalance AIB than the tumour adjacent normal samples (FIG. 5C).

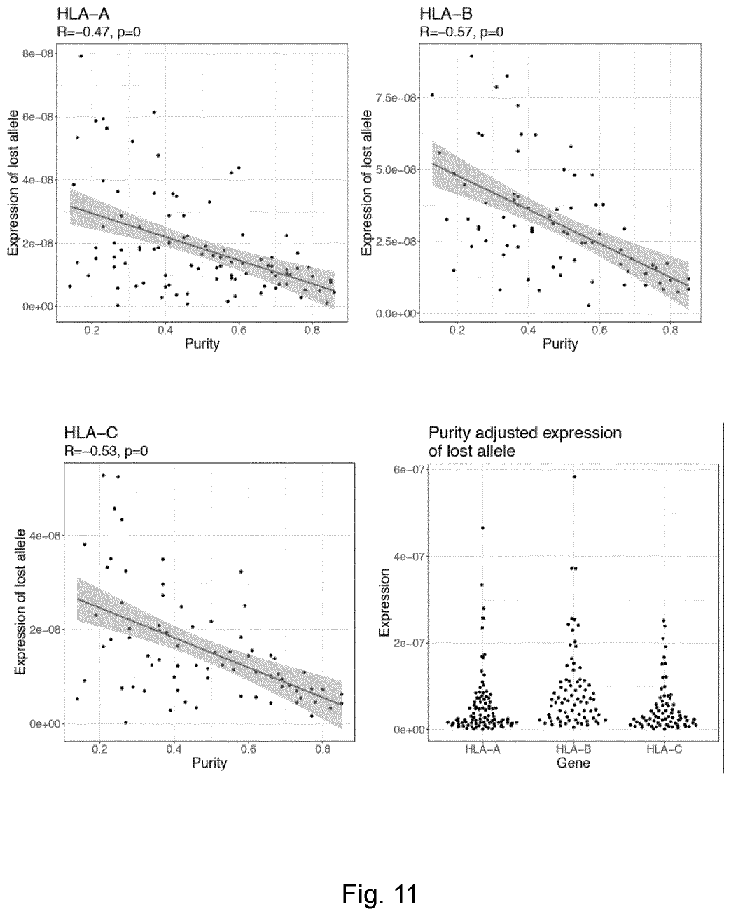

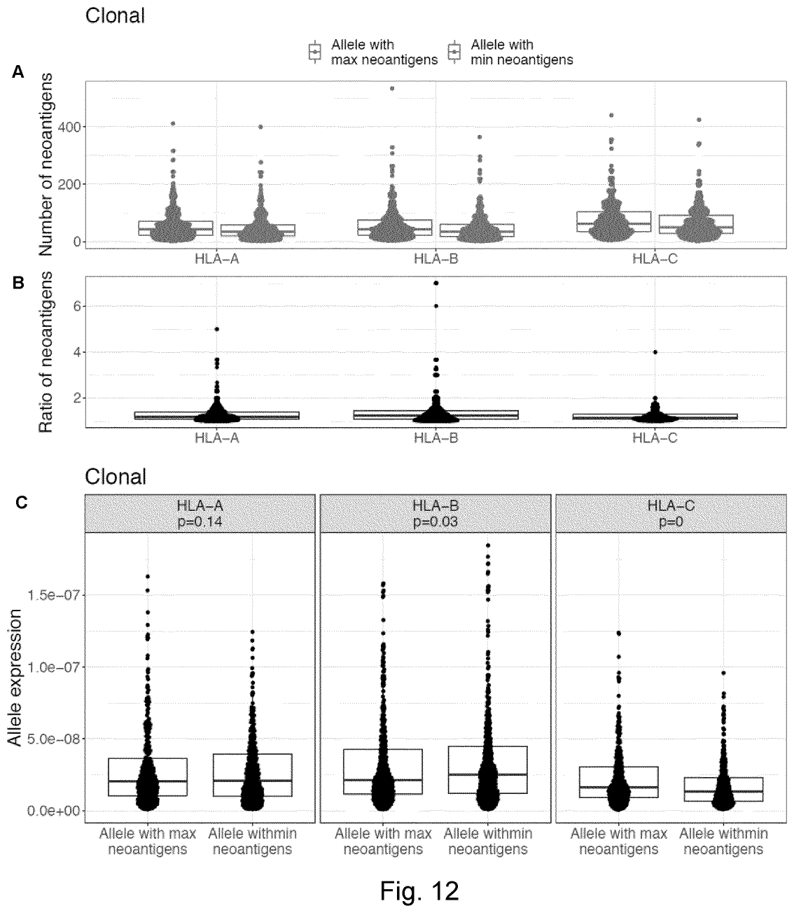

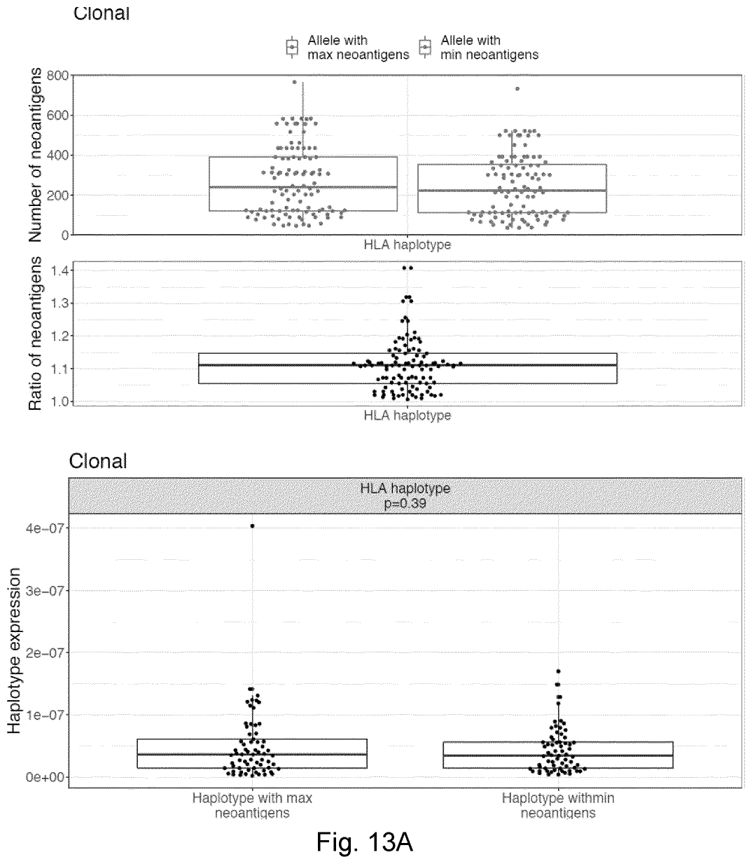

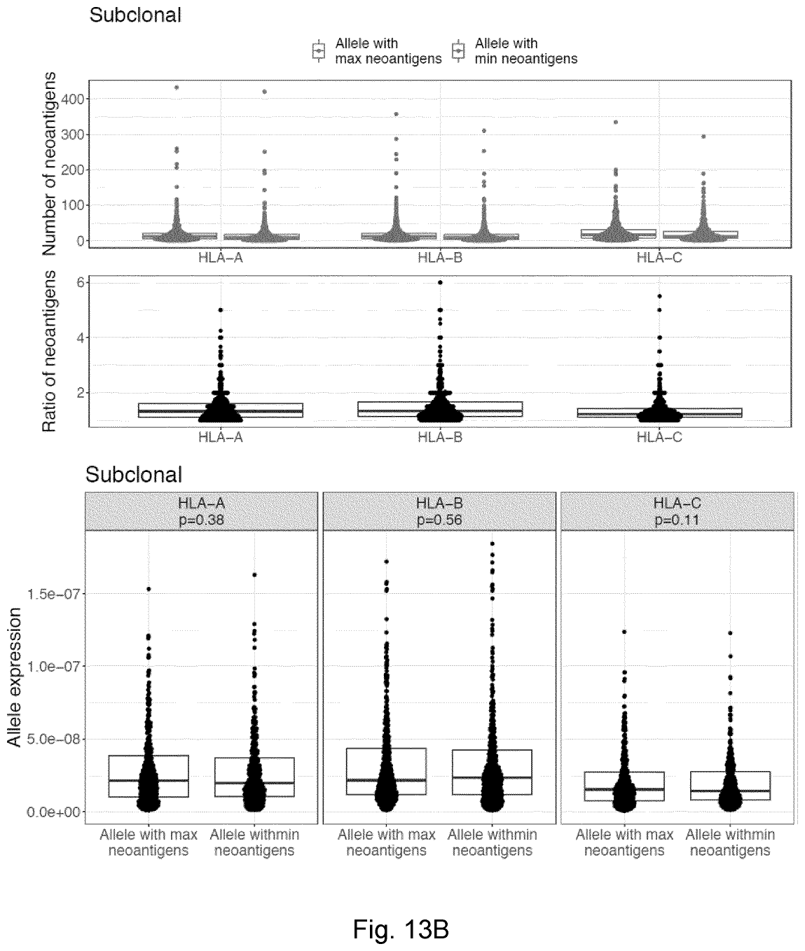

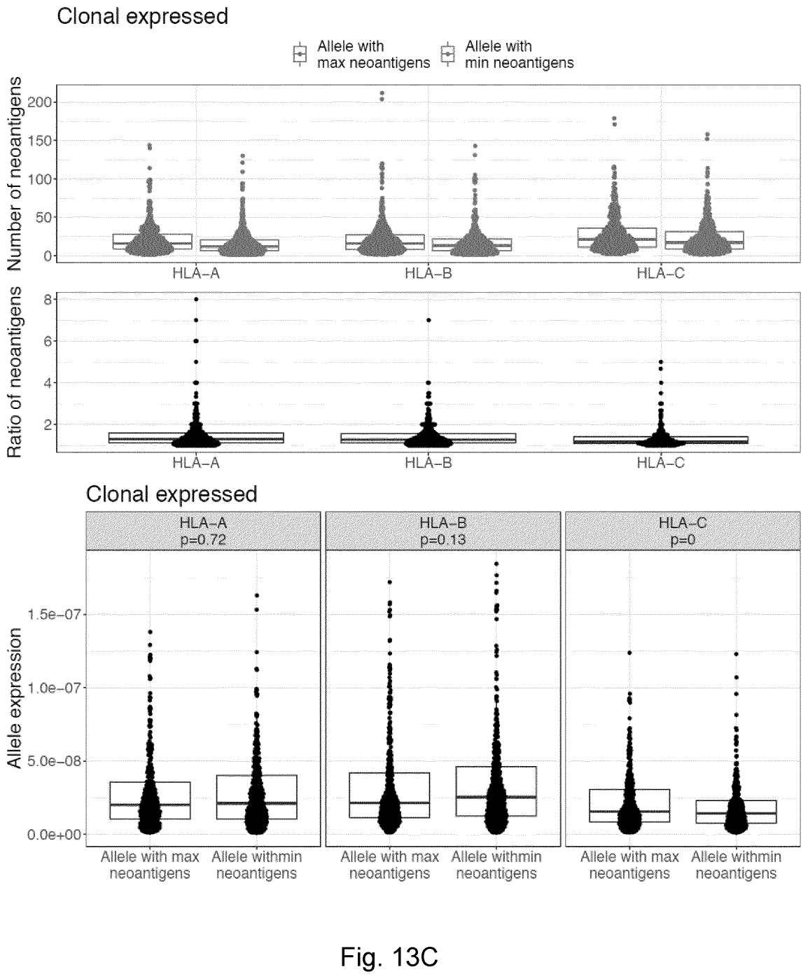

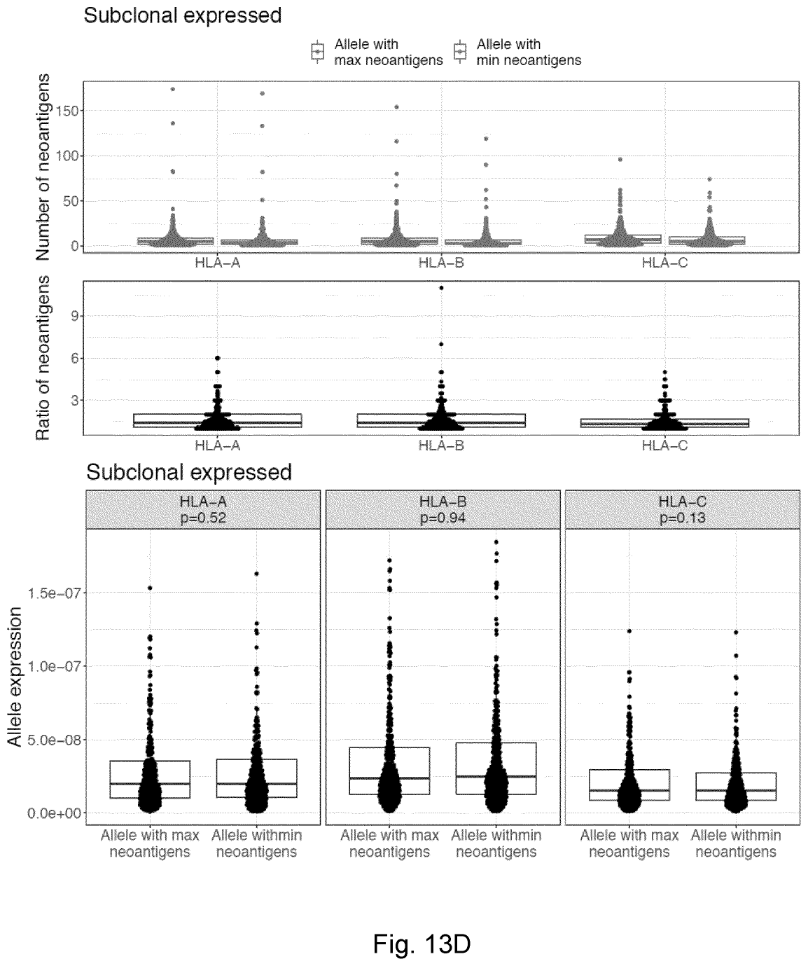

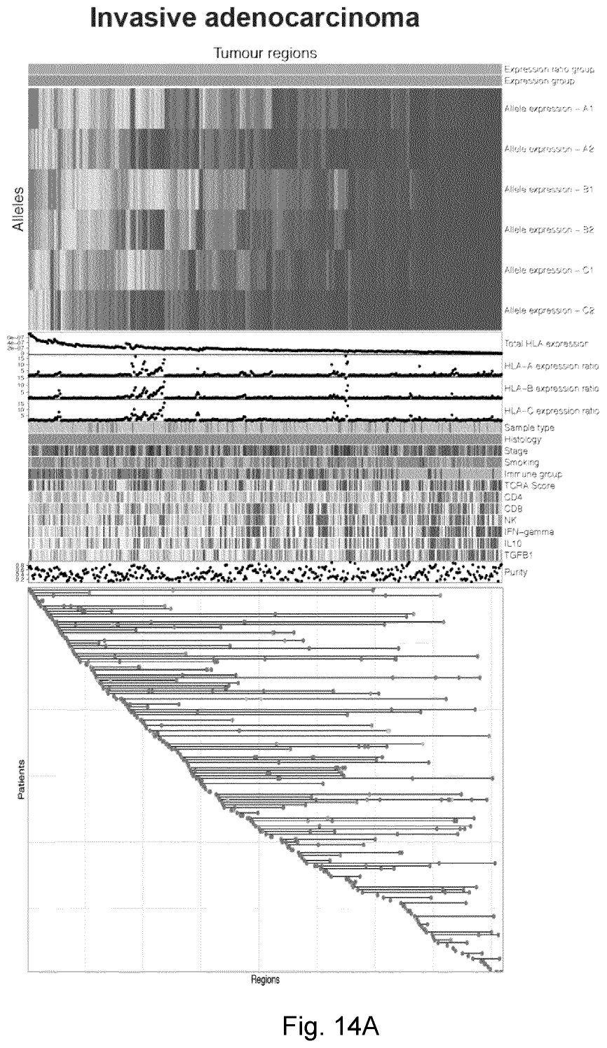

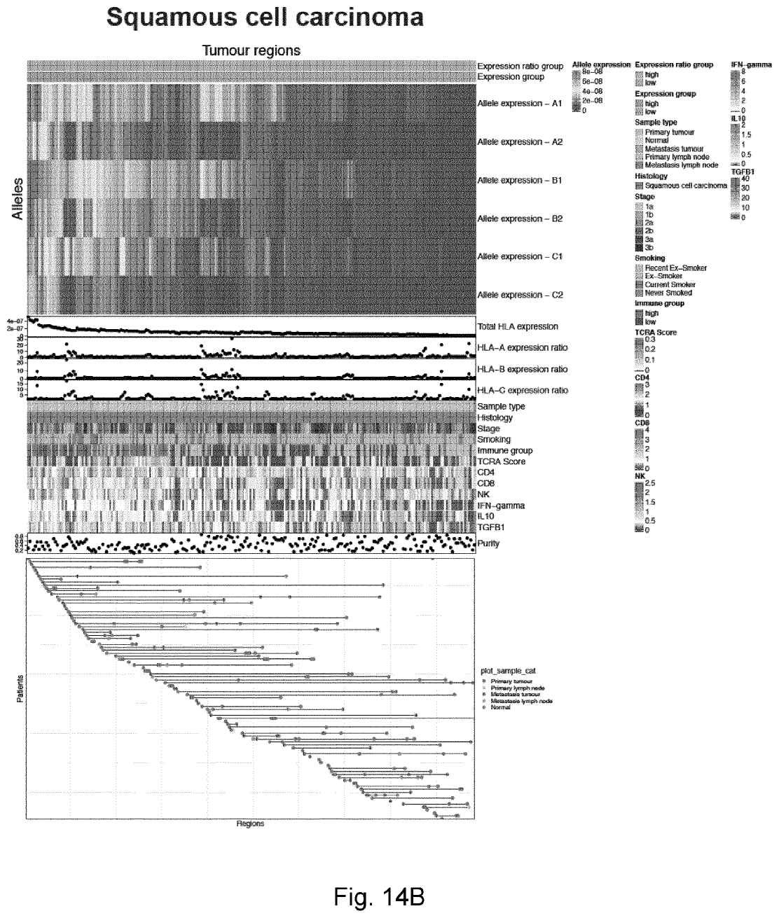

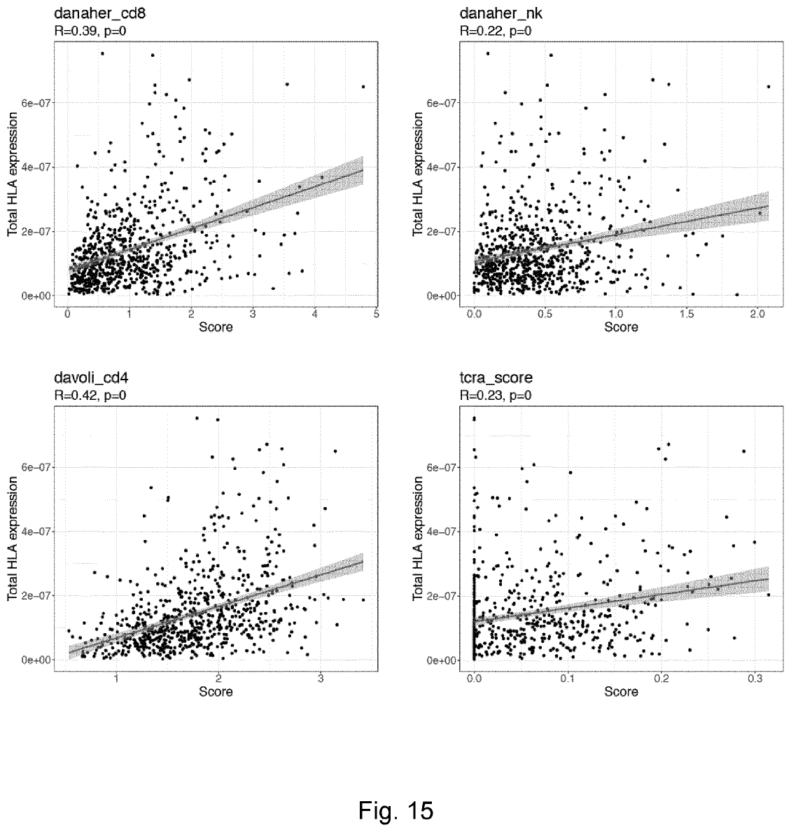

Tumour samples are a mixture of tumour cells, stromal cells and immune cells, all of which can express the class I genes. To investigate HLA expression of the non tumour cells in the tumour regions, we focused on regions with loss of heterozygosity of an HLA class I allele. In this case, any expression of the lost allele must be derived from non cancer cells. As expected, we found a negative correlation between the expression of the lost allele and the tumour purity of the region (FIG. 11). When we performed the same analysis on the kept allele, we also found a negative correlation, however it was weaker. This suggests that in our dataset, class I expression in the non tumour cells is higher than in the tumour cells.

### Measuring HLA Allele Repression in the TRACERx421 Tumour Regions

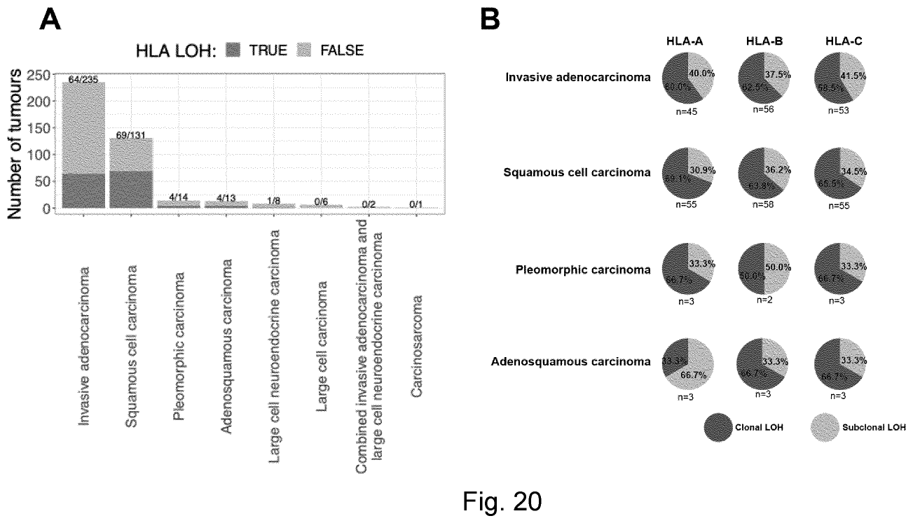

In the TRACERx421 cohort, LOH of the class I HLA genes was frequent, occurring in 64/235 (27.2%) of LUAD, 69/130 (51%) of LUSC primary tumours, and 9/44 (20.5%) of other NSCLC histological subtypes, consistent with our previous findings (FIG. 20A). By contrast, high-impact damaging mutations in the HLA genes were relatively rare, occurring in only 7/410 (1.7%) of primary tumours.

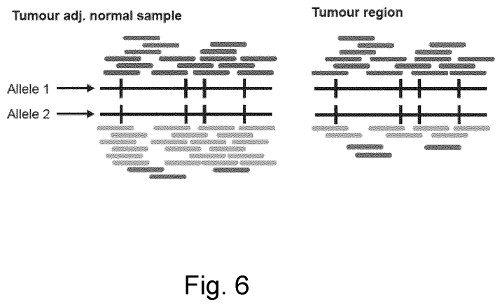

We investigated whether there was evidence of additional transcriptional repression of the HLA alleles in the tumour and paired metastatic regions. Given the high level of heterogeneity observed in HLA allele-specific expression in normal samples, we measured tumour HLA repression with reference to the patient-matched tumour-adjacent normal sample (the “patient-matched normal” approach). We defined an HLA allele as repressed in a tumour region if it had a significantly lower read depth (normalised for library size) at the SNP positions compared to the tumour-adjacent normal sample. In other words, a HLA allele was defined as repressed if the allelic expression was significantly lower in the tumour than the normal, using a paired Wilcoxon test of the normalised depth (read depth divided by the total number of reads in the original BAM file) at the mismatches (FIG. 6). In the present example, a p-value threshold of 0.01 was used. Thus, in the present example, repression was assessed by comparison with a normal sample from the same patient. In total, we were able to evaluate transcriptional repression in 239 primary tumour regions and 17 metastasis regions from 88 tumours (27 LUSC, 48 LUAD, 13 other histological subtypes). However, repression can also be quantified when a matched normal sample is not available, by comparison the expression in the tumour sample to an expected normal level of expression for the allele. Such a comparative normal level of expression can be obtained as a single value or a distribution of values for normal samples with the same allele. The present inventors have identified that individual alleles tend to show a relatively tight distribution of expression values in normal samples (even though wide variability exists between alleles in normal samples), such that comparison with a appropriate value or set of values for the same allele in a normal setting is expected to perform satisfactorily when a matched normal sample is not available. Further evidence of repression in a tumour sample (even in the absence of a normal sample) can be obtained by searching for RNA reads that have one or more somatic mutations identified in the tumour DNA sequencing data.

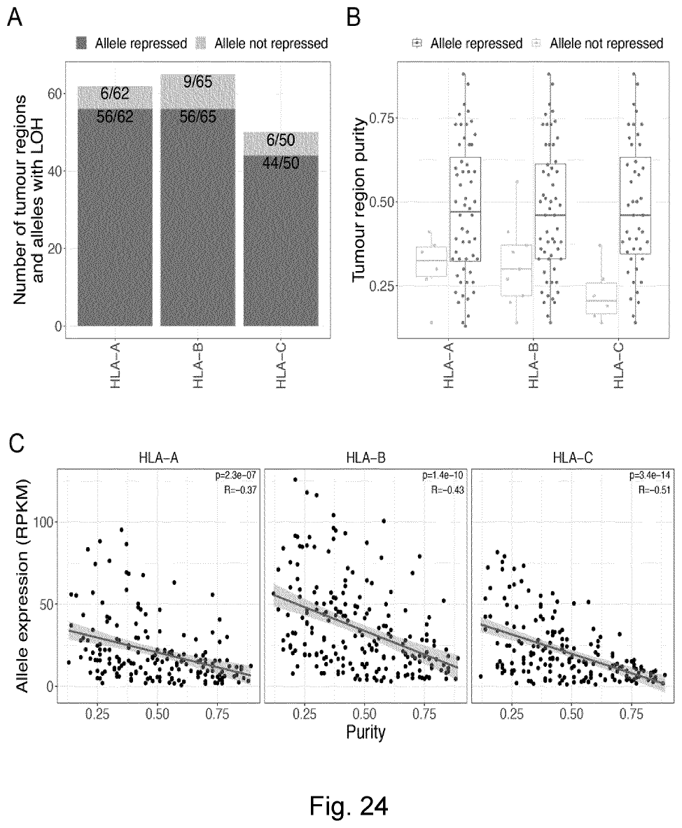

As a control, we checked whether alleles that were predicted to be lost in the DNA were also called as repressed in the RNAseq data, as would be expected. We found 56/62 (90%), 58/67 (87%), and 44/50 (88%) tumour regions predicted to have LOH in HLA-A, B, and C exhibited concordant evidence of HLA repression of the lost allele (FIG. 24A). The few discordant cases had significantly lower purity (p=2.61E-5), making it harder to detect HLA repression (FIG. 24B). Indeed, as expected, we found a significant negative correlation between the allelic expression of the lost allele in the tumour region and the purity of the region (FIG. 24C), illustrating that the non-cancer cells in the tumour regions express class I HLA.

Without wishing to be bound by theory, it is believed that the level of purity that is too low to perform a confident identification of HLA allele specific repression may depend on the sequencing depth of the sample, as well as the specific level of expression to be investigated. A particular level of purity may be determined for a given sequencing depth and expression for example using a training cohort of samples and/or simulated data, by identifying the purity of samples where alleles that were predicted to be lost in the DNA were not called as repressed in the RNAseq data.

We also identified extensive transcriptional repression of the class I HLA alleles that could not be explained by LOH or damaging DNA mutations (FIG. 7A), with 54.1% ( 20/37), 63.4% ( 26/43), and 51.3% ( 17/37) of LUAD tumours, and 52.2% ( 12/23), 68.0% ( 17/25), and 73.9% ( 17/23) of LUSC tumours harbouring repression of HLA-A, B, and C, respectively. 52% of LUAD tumours and 33% of LUSC tumours harboured solely transcriptional repression of HLA with no associated genomic disruption. Taken together, just 21% of LUAD and 7% of LUSC tumours exhibited no LOH or repression in any class I HLA gene (FIG. 4B).

This was also true in the class II HLA alleles (data not shown). Thus, 79% of patients with lung adenocarcinoma and 93% patients with lung squamous cell carcinoma had at least one HLA gene with HLA LOH and/or repression.

As a result of HLA LOH and HLA repression, there are fewer HLA alleles in the transcript, resulting in less HLA alleles that can be presented on the cell surface. This could be an immune evasion mechanism for the tumour regions.

We next quantified the extent of reduction in HLA allelic expression when there was an HLA LOH or repression event. We found that alleles with LOH had a greater reduction in expression than repressed alleles, although this was only significant in LUSC and not LUAD (FIG. 7C) (Wilcoxon test, LUAD: p=0.85, LUSC: p=0.007). These data suggest that while an allelic copy cannot be partially lost in the DNA, it can be partially repressed.

We found that biallelic loss of HLA, resulting in both alleles of the same gene being lost in the DNA, was an uncommon event, occurring in only 3.6% ( 15/412) of NSCLCs, while biallelic loss of all three genes occurred in just a single tumour region in the cohort. To investigate biallelic transcriptional repression, we restricted our analysis to HLA genes with no evidence for LOH. We found that in contrast to HLA LOH, HLA transcriptional repression was more likely to affect both alleles (FIG. 7D). However, while biallelic loss will necessarily impact both alleles equally, we found a significant difference in the tumour-to-normal expression ratio between the two repressed alleles in both LUAD and LUSC (FIG. 7E). This suggests that while both alleles might be repressed relative to normal tissue, they are not repressed equally, conceivably reflecting divergent selection pressures between class I HLA alleles or divergent mechanisms of repression.

We hypothesised that HLA transcriptional repression may be a means to regulate a single HLA gene, whereas HLA LOH may be more likely to disrupt large chromosomal segments, and thus all three class I HLA genes. We found that 65% of HLA LOH events ( 88/136) incorporated all three class I HLA genes. To measure whether the genes are independently transcriptionally repressed, we restricted our analysis to tumour regions with allelic imbalance, but not LOH, across all three HLA genes. Using the allelic copy number, we defined whether alleles were on the major or minor haplotype, and counted, per haplotype, which alleles were repressed. We found that in 46% ( 12/26) of such cases, alleles from all three genes were repressed. These data illustrate that HLA repression occurs less frequently across all three HLA genes compared to HLA LOH.

An individual who is heterozygous for all three HLA genes will have 6 different class I alleles. Each of these alleles may present a different, although overlapping, set of neoantigens to the immune system. To investigate the impact of HLA LOH and transcriptional repression on the predicted number of neoantigens presented to the immune system, we quantified, for each tumour region, the number of different alleles when accounting for: 1) neither LOH nor repression, 2) LOH, or 3) LOH and repression. When accounting for LOH and repression, only 29% ( 38/132) LUAD tumour regions and 6% ( 5/88) LUSC tumour regions had all 6 intact HLA alleles, while 13% ( 17/132) LUAD tumour regions and 19% ( 17/88) LUSC tumour regions had all 6 alleles disrupted at the genomic and transcriptomic levels (FIG. 7F). To estimate the impact of allelic disruption on the number of neoantigens being presented to the immune system, we calculated, for each tumour region, the fraction of putative neoantigens predicted to bind exclusively to alleles subject to LOH or repression. We found that on average, 26.3% (LUAD, interquartile range (IQR) 0-40.9%) and 52.0% (LUSC, IQR 30.3-73.5%) of putative neoantigens were predicted to bind exclusively to alleles subject to LOH or repression (FIG. 7G).

### Investigation of the Mechanisms of Repression

The predominant moderators of HLA gene expression are the NLRC5 protein for the class I genes and the CIITA protein for the class II genes, and it has been shown that disruption to these genes results in lower expression of the HLA genes. We found only 3 tumours that had genomic disruptions (which were all stop gain SNVs) to these genes. This suggests that this is not a common mechanism for repression of the HLA genes in LUAD and LUSC tumours.

### Single Cell Sequencing Data

Current approaches to explore HLA loss and down-regulation developed by us {McGranahan, 2017} and others {Orenbuch et al., 2020}, rely on bulk DNA and RNA-seq data. The advent of novel single-cell sequencing approaches has enabled a more detailed exploration of cancer evolution and intra-tumour heterogeneity {Laks, 2019; Zaccaria 2021}. In particular single cell genomic analysis enables accurate single cell copy alterations to be evaluated. However, there is currently a lack of computational tools or approaches to evaluate HLA loss or down-regulation at single-cell resolution. Here, we propose to adapt our existing tool LOHHLA to enable allele specific copy number and expression estimation of HLA at a single cell level. As described above, the germline BAM is used to determine patient specific HLA alleles, and these are used to investigate the copy number status of HLA haplotypes, for class I and class II alleles. Using single cell data means that the data is typically more sparse than bulk RNA or DNA data. Thus, data across multiple cells may be pooled in order to reach a minimum coverage required to be able to accurately identify HLA type and identify mismatch positions between alleles to quantify expression and LOH. For example, using DLP+ single cell sequencing technology, we sequence an average of 5000 cancer cells per sample. Thus assuming 0.025× per single cancer cell, we obtain an effective bulk coverage of 125× (5000×0.025=125×). Thus, a “pseudo-bulk” approach combining a subset of these cells should provide more than enough coverage for HLA typing and allele specific copy number calling as coverage as high as 125× is not required for this.

### Example 2—Evaluation of HLA Splicing in Lung Cancer and Normal Tissue

The inventors developed a method to evaluate alternative splicing, such as exon skipping, by identifying high-quality sequencing reads providing evidence of a skipping event.

The classical class I HLA genes are organised into seven (HLA-B) or eight (HLA-A and HLA-C) exons, each with distinct functional domains. Exon 1 encodes the signal peptide, while exons 2, 3, and 4 encode the α1, α2, and α3 domains respectively. The α1 and α2 domains make up the peptide binding region, while α3 binds to a β2 microglobulin protein (encoded on chromosome 15) to stabilise the HLA molecule. The α3 domain also binds the CD8 co-receptor, strengthening CD8+ T cell activation. Exon 5 encodes the transmembrane domain and the cytoplasmic tail is encoded by the remaining exons (FIG. 17A). Thus, alternative splicing may yield an altered yet functional protein, or introduce a premature termination codon (PTC) that results in a truncated protein or nonsense-mediated decay (NMD), all of which could conceivably disrupt HLA presentation and limit tumour immunogenicity.

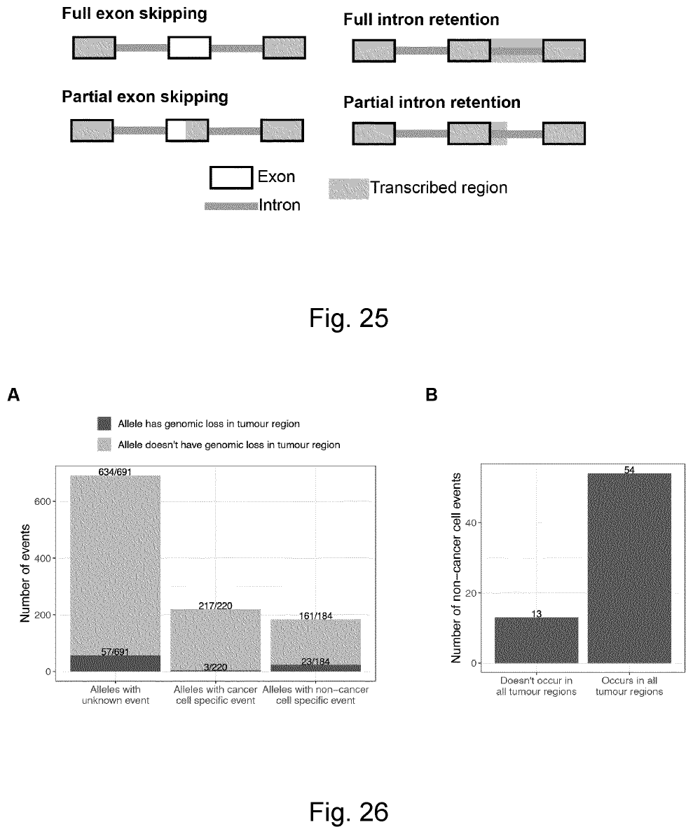

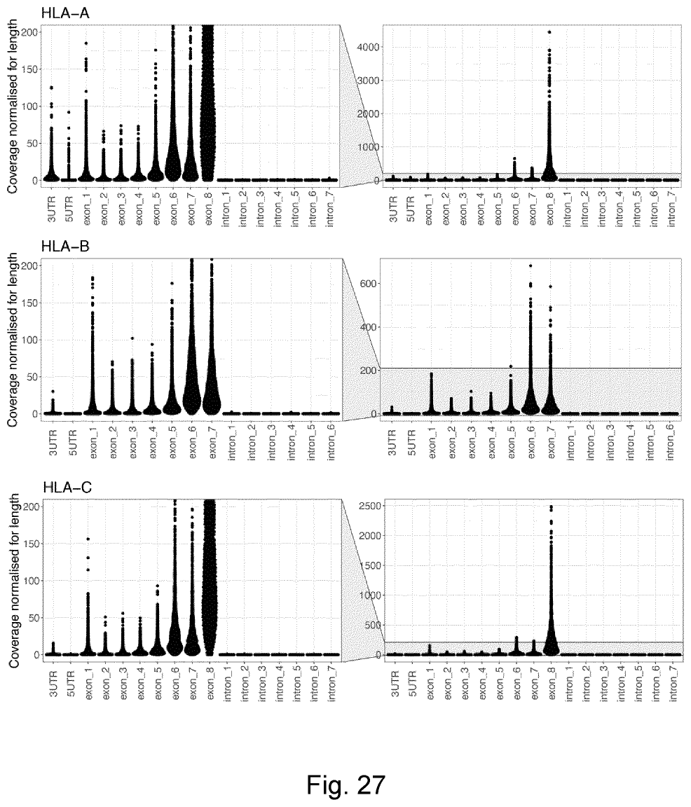

Given the role that HLA alternative splicing could play in tumour immune evasion, we used MHC Hammer to investigate the prevalence of HLA alternative splicing in the 88 patients with a matched tumour-adjacent normal sample. MHC Hammer can identity four different types of alternative splicing events: 1) complete exon skipping, which results in the given exon being absent from the mature mRNA transcript; 2) partial exon skipping, which results in some, but not all, of the given exon being absent in the mature mRNA transcript; 3) partial intron retention, which results in some, but not all, of the given intron being retained in the mature mRNA transcript and 4) complete intron retention, which results in a complete intron being retained in the mature mRNA transcript (FIG. 25). In our cohort, MHC Hammer identified full exon skipping, partial exon skipping, and partial intron retention in the HLA alleles in both the tumour regions and tumour-adjacent normal samples. We did not observe evidence for full intron retention in any HLA allele (FIG. 27). We validated our alternative splicing calls using polymerase chain reaction (PCR) with an allele-specific primer.

As explained in the Methods, the following process is used to identify reads that provide evidence of alternative splicing Only reads that map (exact match) to any k-mer from the patient specific reference (where a k value of 30 was used in this work) were selected from the RNAseq BAM files. For every allele locus, the patient specific reference (allele specific genomic fasta file and GTF file indicating the locations of introns and exons) was provided together with these selected reads to STAR. We ran STAR in a two pass alignment mode (as described in Veeneman et al., 2015). This involves running STAR once on a tumour region basis, to get the location of novel splice sites in each allele (novel splice sites are those that are not in the GTF files, e.g. known intron/exon boundaries.) Then, for each allele in the cohort, we collate all the novel splice junctions observed in the first run of STAR. We the rerun STAR on a tumour region basis, but also input the novel splice junctions identified in the first step. This two-step process is designed to make the pipeline more sensitive to novel splice junctions. After the second run of STAR, we filter the novel splice junctions to only keep those that have at least 20 uniquely mapping reads supporting them.

The number of such high-quality selected reads that support the existence of an exon skipping event in a particular allele (e.g. reads that map to exons 4 and 6 but not exon 5) was used as a metric of exon skipping. An exon was considered skipped if a minimum number of reads (20 reads, in this example) were identified. A metric based on the ratio of reads that do and do not support the existence of an exon skipping event was also tried. In particular, the ratio compared the number of reads that map to surrounding exons but not to a candidate skipped exon, and the mean of the number of reads that map to both a surrounding exon and the candidate skipped exon. This metric was not used because it could be skewed by the existence of strongly favoured splice junctions. Further, exon skipping events can happen at very low frequency and it is advantageous not to filter out such low frequency events if they have good support in the data.

To further evaluate HLA alternative splicing, we grouped events into one of three categories: non-cancer cell specific, defined as events that were also detected in the patient-matched tumour-adjacent normal sample; somatic, defined as events that were not detected in the matched tumour-adjacent normal sample; and, unknown, describing alternative splicing events detected in tumours without a matched tumour-adjacent normal. We reasoned that somatic alternative splicing events that were called in alleles that were also predicted to be subject to deletion, resulting in LOH, must reflect false positives (or an erroneous LOH call). Reassuringly, we only observed 3/220 somatic events in regions where the allele was also predicted to exhibit a genomic loss (FIG. 26A). Further, we would expect any non-cancer cell specific event to generally be present ubiquitously in all the tumour regions, provided the allele was not subject to deletion or repression. We found that 54/67 (81%) non-cancer cell specific events occurred ubiquitously (FIG. 26B).

In total, we observed somatic alternative splicing events in exons 2, 3 or 4 or in introns 2, 3 or 4, in 39.6% of LUAD tumours and 25.9% of LUSC tumours (FIG. 18B). This included complete skipping of exon 3 in 20.0% of NSCLCs and partial skipping of exons 2, 3 and 4 in 5.3%, 4.0%, and 16.0% of NSCLCs. We observed cancer cell specific partial intron retention of introns 3 and 4 in 1.3% and 2.7% of NSCLCs. Due to the structure of the HLA molecule, changes to the amino acid sequence that are encoded by exons 2, 3 and 4, through exon skipping or intron retention, could result in an unstable HLA molecule potentially unable to present neoantigens to the immune system (Reinders et al., 2005).

We also observed somatic complete exon 5 skipping in 25.0% of LUAD and 22.2% of LUSC tumours, partial exon 5 skipping in 12.5% of LUAD and 3.7% of LUSC tumours, and partial intron 5 retention in 27.1% of LUAD and 33.3% of LUSC tumours (FIG. 18B). Alternative splicing resulting in exon 5 skipping has been shown to result in a soluble HLA allele (Dubois et al., 2004; Krangel 1986). Soluble HLA class I molecules could provide a mechanism of immune tolerance by delivering a neoantigen signal to the T cell receptor, distant from the cancer cell, without costimulatory or accessory signals. Alternatively, persistent neoantigen presentation to T cells could lead to T cell exhaustion.

Across the cohort, we found cancer cell specific alternative splicing of the HLA alleles to be a common event in both major histological subtypes, with 58.3% of LUAD and 51.9% of LUSC tumours harbouring at least one somatic alternative splicing event, and 39.6% LUAD and 29.% of LUSC tumours harbouring multiple somatic alternative splicing events, including one LUAD tumour harbouring 9 different events (FIG. 18C).

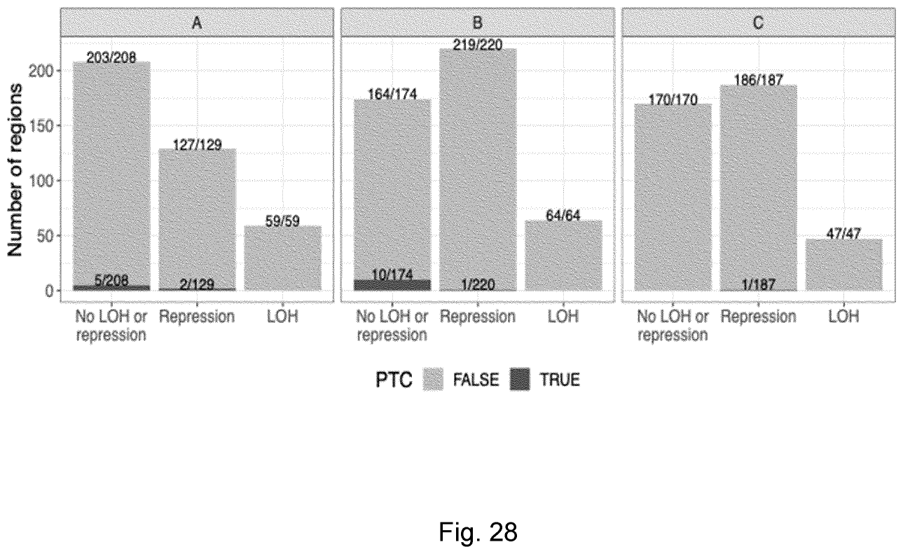

The introduction or deletion of amino acids due to an alternative splicing event could result in a frameshift and/or the introduction of a PTC (premature termination codon) in the resulting transcript. We found that all complete exon skipping events were inframe, while partial exon skipping and partial intron retention occurred inframe, out-of-frame without a PTC, and out-of-frame with the introduction of a PTC (FIG. 18D). LUSCs were more likely to harbour inframe partial exon skipping or intron retention events than LUAD tumours. Previous work has found that the introduction of a PTC in exons 2, 3 and 4, and in the 5′ end of exon 5 in HLA-A results in a reduction of mRNA expression due to NMD (Watanabe et al., 2001). However, we did not find evidence that alleles predicted to be transcriptionally repressed were more likely to have alternative splicing-induced PTCs (FIG. 28).

Given that tumour samples reflect an admixture of cancer cells and non-cancer cells, to estimate the fraction of alternatively spliced transcripts in the cancer cells, we scaled the ratio of novel-to-canonical transcripts (the novel transcript ratio) by the purity of the tumour region (Supplementary methods). Inframe events had the highest purity-scaled novel transcript ratio, followed by out-of-frame events without a PTC, and then out-of-frame events that introduced a PTC (FIG. 18E). The purity-scaled novel transcript ratio was less than 10% in the majority of cases. These data suggest either one or both of the following are occurring: within each cancer cell, both the canonical and novel transcripts are being transcribed, or only a subset of cancer cells harbour the novel transcript.

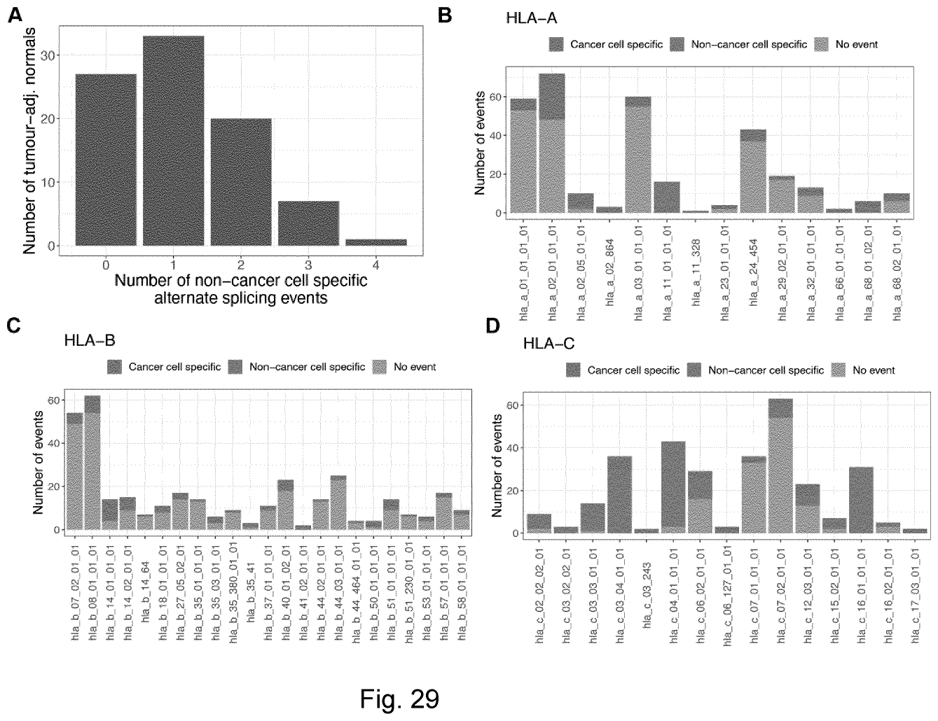

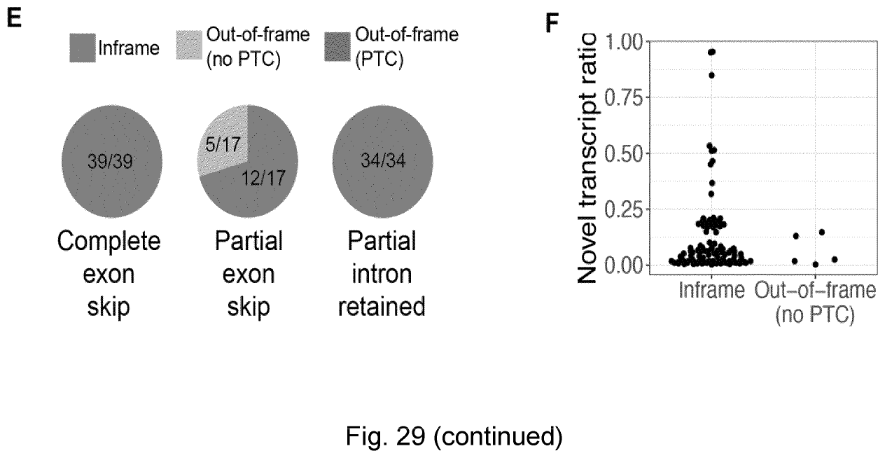

We identified non-cancer cell specific HLA alternative splicing in 69.3% ( 61/88) of tumours with a tumour-adjacent sample, of which 31.2% ( 28/88) exhibited multiple non-cancer cell specific alternative splicing events (FIG. 29A). In contrast to somatic alternative splicing, these events were enriched for specific alleles, such as HLA-A*11:01:01:01 and HLA-C*03:03:01:01 (FIG. 29B,C, D). Amongst them, 39/39 complete exon skipping, 34/34 partial intron retention, and 12/17 partial exon skipping events were inframe, whilst the remaining 4/17 partial exon skipping events were out-of-frame but did not introduce a PTC (FIG. 29E). The novel-transcript ratio for non-cancer cell specific alternative splicing events ranged from 0.4 to 95%, with an average of 11% (FIG. 29F).

To further evaluate the rate of cancer cell specific alternative splicing observed in HLA alleles, and whether this is higher or lower than might be expected, we considered the rate of alternative splicing in established cancer genes. In brief, we adapted our HLA alternative splicing pipeline to explore somatic alternative splicing events across 130 lung cancer oncogenes and tumour suppressor genes defined by previous studies (Bailey et al., 2018; Berger et al., 2017; Martincorena et al., 2018). When considered in the context of other oncogenes and tumour suppressor genes, we found that HLA-A had the 3rd, HLA-B the 5th and HLA-C the 8th highest frequency of alternative splicing (FIG. 18F). These data suggest that alternative splicing of the HLA alleles is likely positively selected for in tumour evolution.

Consistent with selection of alternative splicing events, we observed that tumour regions without disruption of HLA expression through LOH or repression were significantly enriched for cancer cell specific alternative splicing events (p=1.4E-8, Fisher's exact test) compared to regions that harboured either HLA LOH or repression (FIG. 18G). This suggests that cancer cell specific alternative splicing offers an alternative means to disrupt HLA presentation during lung cancer evolution.

To further investigate the importance of HLA disruption via alternative splicing, we compared the total number of neoantigens predicted to bind only to alleles with or without cancer cell specific alternative splicing affecting either exons/introns 2, 3 or 4, or exon/intron 5. We did not find a significant difference with alleles that had alternative splicing affecting exon/intron 5. However, the alleles with alternative splicing affecting exons/introns 2, 3 or 4 in LUAD tumours were predicted to bind a significantly higher number of neoantigens, compared to alleles without (p=5.83E-5, FIG. 18H) suggesting that alternative splicing of HLA alleles may be selected to reduce antigen presentation.

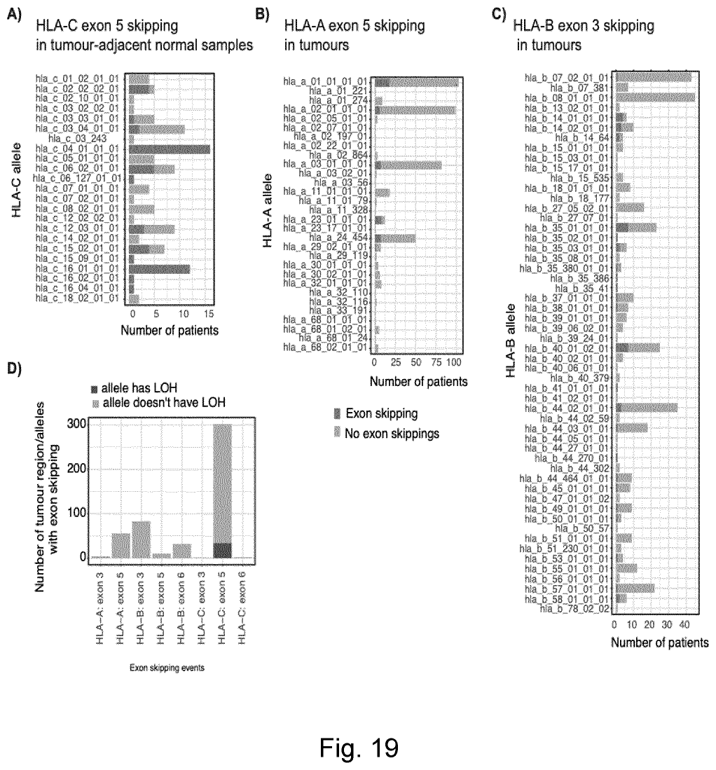

As can be seen in FIG. 18A, we observe sequencing reads supporting exon skipping of exons 3, 5 and 6 in both tumour regions and tumour-adjacent normal samples. Our data suggest that skipping of exon 5 in HLA-C is not a tumour specific event, while skipping of exon 3 and exon 5 in HLA-A or HLA-B is a tumour specific event. Exon 5 skipping in HLA-C is often observed in the tumour adjacent normal tissue (FIG. 18A), and specific HLA-C alleles are more likely to exhibit HLA-C exon 5 skipping (e.g. HLA-C 04:01:01:01, as has been described before) (FIG. 19). By contrast, we never observe skipping of exons 3 or 5 in HLA-A or HLA-B in tumour adjacent normal tissue (FIG. 18A), and no specific allele alleles are more likely to exhibit exon skipping (FIG. 19). Finally, within tumour regions we also observe skipping of HLA-C exon 5 in alleles that are predicted to be lost through DNA copy number loss, suggesting that this event cannot be exclusive to cancer cells. This is in contrast to exon 3 and 5 skipping in HLA-A or HLA-B, which is never observed in alleles that are lost due to an LOH event (FIG. 19D).

Exons 2 and 3 encode the peptide-binding groove, and, as such, alternative splicing of these exons may directly disrupt HLA presentation. As our results suggest that exon skipping of exons 3 is tumour specific, this is consistent with the notion that this may be selected to help facilitate immune evasion. Alternative splicing resulting in skipping of exon 5 has been shown to result in a soluble HLA allele {Dubois, 2004}. Conceivably, secreted HLA class I molecules could provide a mechanism of tolerance by delivering a signal to the T cell receptor, distant from the cancer cell, without costimulatory or accessory signals. Interestingly, we observe skipping of HLA-C exon 5 in both tumour and tumour-adjacent normal tissue, suggesting that while this may be important for tumour evolution, it is not specifically selected solely in the context of tumour development.

A critical next step is to validate our exon skipping observations. This was performed using long-read sequencing data RNA-sequencing data, making use of the oxford nanopore sequencing platform. Long-read sequencing provides sequencing reads that are >200 bp in length. These can therefore be used to obtain closer to full length transcripts of HLA alleles. Using long-read sequencing enabled to evaluate the extent of exon-skipping and also whether this is associated with upstream mutations. Additional validation by PCR was performed. In particular, PCR validation followed the method in Gerritsen 2016, using rtPCR and analysis of rtPCR products through electrophoresis gels. In such a protocol, if there is an exon skipping event then one would expect to see two bands in the gel, one representing the transcript with the exon and one without the exon. Initially, skipping of HLA-C exon 5 was investigated. 10 different normal samples and tumour regions with/without exon 5 skipping event with a range of different HLA-C alleles were analysed using rtPCR amplification with two different pairs of primers: Exon 1 and 3′UTR, and 5′UTR and 3′UTR. This revealed that the presence/absence/strength of the band depended on the patients HLA-C allele type such that allele specific primers should ideally be used. The amplification was rerun using two different pairs of primers: HLA-C*07 exon 1 and 3′UTR, and HLA-C*16 exon 3 and 3′UTR (see Gerritsen, Table 1 A and Tale 1B). This showed two clear bands for samples in which exon skipping was expected (HLA-C*16). The presence of an exon skipping event can be further validated at the protein level using Western Blot.

The detection of the presence of an exon skipping event as described herein can be used to evaluate the mechanisms underpinning this exon skipping (e.g. investigating whether a somatic mutation may have caused the appearance of a novel splice site), and further, evaluate its impact on the immune microenvironment. For example it becomes possible to explore the extent to which the number of predicted mutant peptides binding to a specific HLA allele relates to the likelihood of observing alternative splicing and exon skipping. As another example, these results enabled to determine whether HLA splicing is restricted to specific HLA types. It was found that if the splicing is in the normal sample then it is more common in certain allele types. However, this is not the case if it is only observed in the tumour regions. Further, this pipeline can be extended beyond class I HLA alleles to also evaluate exon skipping and alternative splicing of class 1l, nonclassical class I and II genes, as well as TAP1/2 and B2M.

Ultimately, this enables a detailed exploration of the importance of alternative splicing as a mechanism of immune evasion and antigen presentation disruption in lung cancer evolution

### Example 3—HLA Disruption and Tumour Evolution

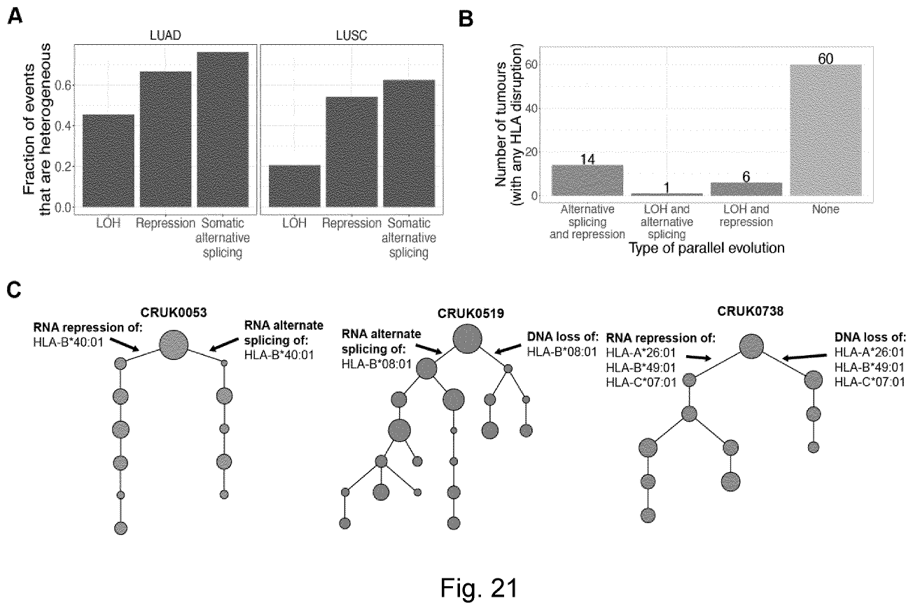

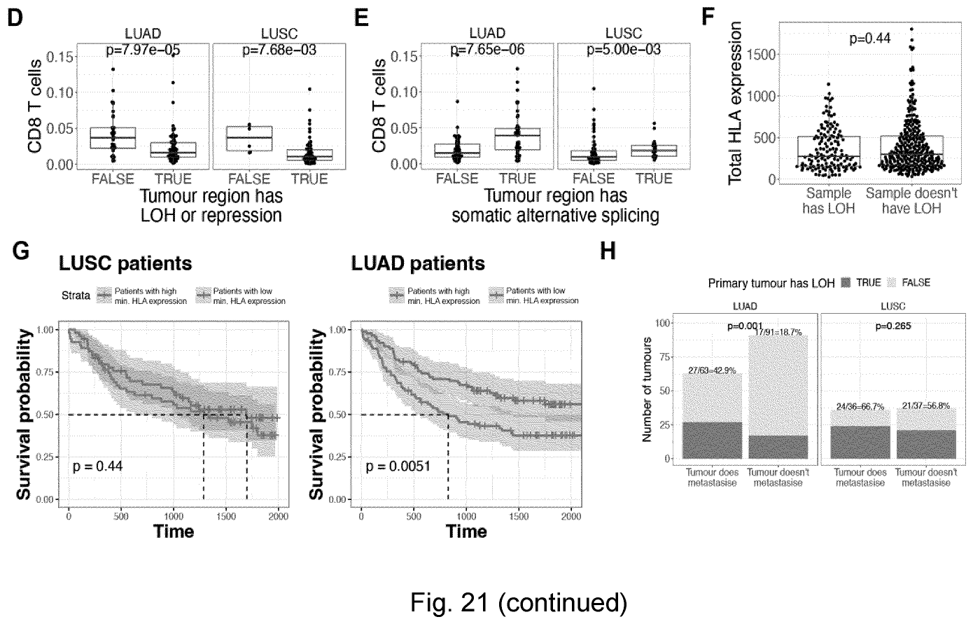

To understand when HLA LOH, transcriptional repression and somatic alternative splicing occur during NSCLC evolution, we considered the heterogeneity of these events. We defined an HLA LOH, repression or somatic alternative splicing event as ubiquitous if it occurred in all of the primary tumour regions, and heterogeneous otherwise. We found that somatic alternative splicing events were the most heterogeneous, followed by repression and then LOH (LUAD LOH: 45.5%, repression: 66.7%, alternative splicing: 76.2%; LUSC LOH: 20.5%, repression: 54.2%, alternative splicing: 62.5%) (FIG. 21A).

In 21/81 (25.9%) tumours with HLA disruption, we observed convergence upon disruption of the same allele through alternative mechanisms; with genomic loss, transcriptional repression and/or alternative splicing of the same allele occurring in different regions of the same tumour. In particular, we observed 6 tumours with convergence upon genomic loss and transcriptional repression of the same allele in separate regions, 14 tumours with transcriptional repression and alternative splicing of the same allele in separate regions and 1 tumour with genomic loss and alternative splicing of the same allele in different regions (FIG. 21B).

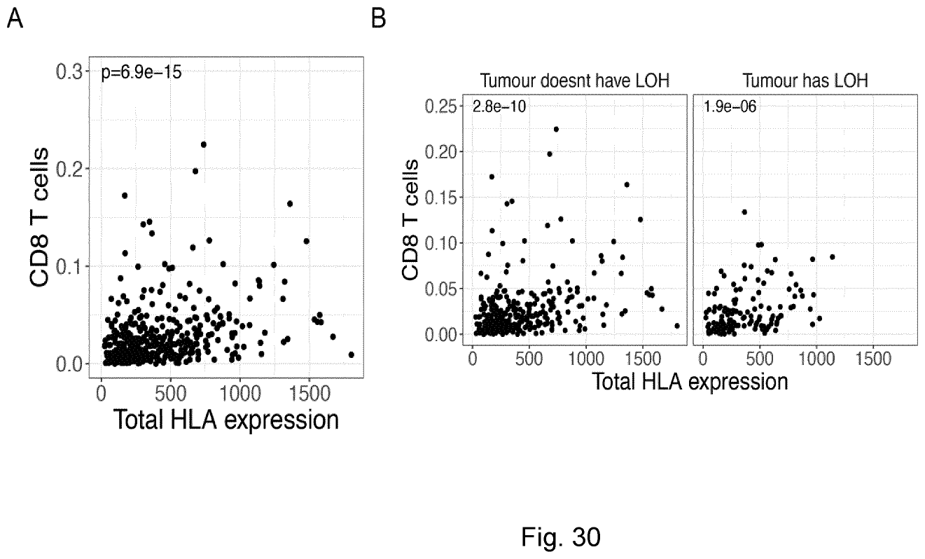

It is well known that the tumour microenvironment can shape tumour evolution (Rosenthal et al., 2019). We therefore investigated the relationship between the immune infiltrate and the presence of HLA disruption. Using CIBSERORTx (Newman et al., 2019), we observed a significant relationship between total HLA expression and CD8 T cell infiltrate (p=6.9E-15, FIG. 30A). In particular, we observed that tumour regions with allelic HLA transcriptional repression—linked to lower total HLA expression—had lower levels of infiltrating CD8 T cells compared to those without (LUAD p=8.0E-5, LUSC p=7.7E-3, FIG. 21D). Conversely, HLA alternatively splicing—linked to higher total HLA expression—was associated with elevated CD8 T cell levels (LUAD p=7.7E-6, LUSC p=5.0E-3, FIG. 21E). We did not observe a clear relationship between HLA LOH and total HLA expression, indicating dosage compensation may occur following allelic HLA copy number loss (FIG. 21F). However, both for tumours with and without HLA LOH, a significant positive relationship between total HLA expression and CD8+ T cell infiltrate was observed (FIG. 30B).

We next investigated the association of HLA expression with patient outcome. We calculated the total HLA expression across all 6 alleles for each tumour region, and then calculated the maximum and minimum total HLA expression across all regions for a given tumour. We found that in LUAD patients, the minimum HLA expression across the tumour regions predicted survival, with tumours with high HLA expression across all regions associated with the best disease-free survival outcomes (FIG. 21E).

Finally, we endeavoured to understand whether disruption of the HLA alleles, through LOH, repression, or alternative splicing might play a role in the evolution of lung cancer metastasis. We found that LUAD tumours that harboured HLA LOH were more likely to metastasise than those without HLA LOH (p=0.008, FIG. 21F). LUSC primary tumour regions that seed metastases tended to have lower total HLA expression compared to regions that did not seed metastases (LUSC p=0.04, LUAD p=0.15). Taken together, these data suggest that disruption of the HLA alleles could play an important role in tumour metastasis.

### Example 4—Prediction of Response to Immune Checkpoint Blockade

Previous work has shown that in a non-small cell lung cancer cohort, taking HLA LOH into account when calculating TMB can improve response prediction {Shim, 2019}. However, to date, neither the expression of the HLA locus nor the copy number of putative neoantigens has been incorporated into models to predict response to immune checkpoint blockade.

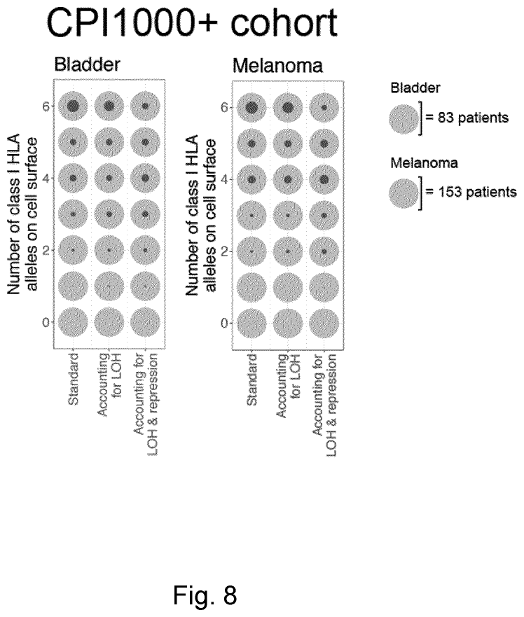

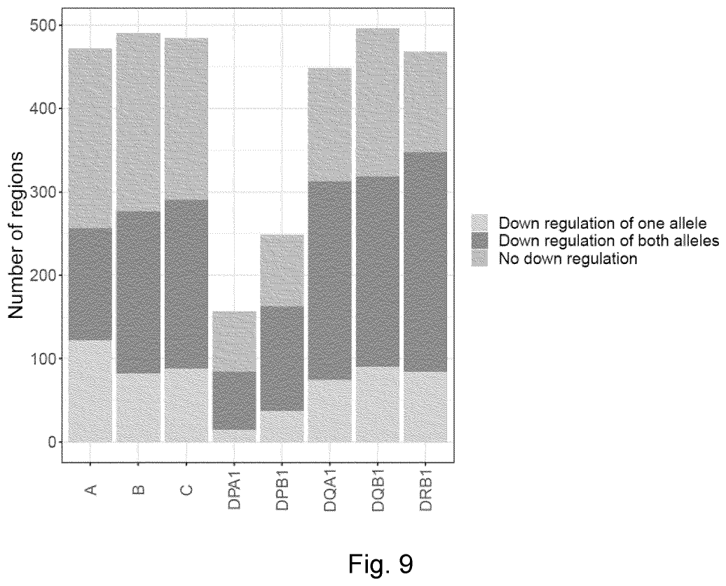

Our preliminary analysis (FIG. 8) illustrates the number of alleles that would be presented on the cell surface, after accounting for LOH and repression, in both a Melanoma and a Bladder cohort {Litchfield, 2021}. This suggests that in many tumours we are overestimating the putative neoantigen burden, and that an approach that corrects for HLA expression and copy number may yield an improved understanding of the determinants of response to immune checkpoint blockade. The inventors used a large uniformly processed dataset of samples subject to immune checkpoint blockade {Litchfield, 2021}, as well as TRACERx tumours subject to immunotherapy to explore this further. They showed that an “effective neoantigen burden” can be used to predict immune infiltrate (using the Danaher scores from RNAseq data and/or the TCRA score from Bentham et al., 2021, they investigated whether there was a relationship between the effective neoantigen burden and the immune infiltrate) and response to treatment. This may have important implications in designing vaccine therapies.

Thus, the data shows that HLA dysregulation reduces the number of alleles on the cell surface and neoantigens presented to T cells, which can be taken into account to predict response to immunotherapy.

### Discussion

Neoantigen presentation via the HLA molecules is crucial to achieve an antitumour immune response. Previous studies have illustrated that different mechanisms of HLA disruption are common across cancers. These examples describe and demonstrate the use of a bioinformatics tool to investigate the prevalence of four mechanisms of genomic and transcriptomic disruption of the HLA alleles in NSCLC: mutations, LOH, repression, and alternative splicing.

We found that while damaging HLA mutations were not common in our cohort, LOH, repression, and cancer cell specific alternative splicing of the HLA alleles was pervasive. From the patients with tumour-adjacent normal samples, just 7% of LUSC and 23% of LUAD tumours had no HLA LOH or repression, while 58.8% of LUAD and 50.0% of LUSC tumours harboured at least one cancer cell specific alternative splicing event.

One limitation of our method is that it requires a patient-matched tumour-adjacent normal tissue sample to call HLA repression and cancer cell specific alternative splicing (though not necessarily to call alternative splicing without cell type specificity). This is due to the heterogeneity that we observed in HLA allelic expression in the normal tissue samples, and the fact that we observed HLA alternative splicing in normal tissue samples. Indeed, alternative splicing of the class I HLA alleles has been observed in specific alleles in non-cancer tissue and in some cancer cell lines. However, to the best of our knowledge, cancer cell specific HLA alternative splicing in a large cohort of patients has not been described before, and may therefore represent a novel mechanism of immune evasion.

HLA alternative splicing affecting exons or introns 2-4 could result in an unstable HLA molecule. For example, partial exon 3 skipping in an HLA-A allele in non-cancer tissue has been shown to result in the absence of cell surface expression (Reinders et al, 2005). In another case, an HLA-A allele with full exon 3 skipping continued to be expressed on the cell surface, but as an immature glycoprotein unable to present peptides (Dai et al., 2014). This immature molecule could potentially act as a decoy allele by inhibiting NK cells via its receptor ligands without presenting neoantigens to CD8 T cells.

Alternative splicing resulting in exon 5 skipping has been shown to result in a soluble HLA allele. Persistent presentation of neoantigens via soluble HLA molecules to the T cell receptor, without costimulatory or accessory signals, could lead to immune tolerance or T cell exhaustion. It has been shown that soluble class I HLA molecules can induce apoptosis in CD8 T cells and NK cells (Contini et al., 2003). We also observe cancer cell specific partial exon 5 skipping and partial intron 5 retention in the HLA alleles, however it is not clear whether these events would sufficiently disrupt the transmembrane domain to result in a soluble HLA molecule (Tijssen et al., 2000).

In comparison to HLA mutations and LOH events, it is possible that HLA repression and alternative splicing events are transitory, where their presence or absence may fluctuate throughout the tumour's evolution. In support of this, we found that HLA repression and alternative splicing events were more likely to be heterogeneous than LOH events.

Thus, the data demonstrates that transcriptional deregulation of HLA alleles is a crucial part of HLA deregulation in tumour, and that HLA LOH only captures part of the picture in terms of HLA deregulation. Further, this work demonstrates that the use of a patient specific genomic reference sequence for the HLA to map transcriptomics data advantageously enables the determination of a complete picture of transcriptional deregulation including both repression and exon skipping. Additionally, in relation to transcriptional repression, the data shows that accurate assessment of this requires the use of a comparative normal level of expression as highly variable levels of expression are possible for different alleles in normal tissues. Further, the data shows that alternative splicing of HLA alleles (e.g. exon skipping of HLA class I alleles exons 3, 5 and 6) can be evaluated based on short read data using high quality reads that contain evidence of a skipping event. This reveals a picture where alternative splicing such as exon skipping is common in tumour and potentially underlines complex mechanisms of immune evasion. Finally, the data shows that taking into account transcriptional repression when assessing tumour mutational burden enables to more accurately estimate this important diagnostic and prognostic metric, and thus enables to better predict response to immunotherapy such as CPI therapy, and that expression of HLA alleles is predictive of survival in at least some types of cancers.

As more data pre- and post-therapy emerges, it will be possible to investigate the extent to which HLA alternative splicing and repression develop during treatment, and the extent to which they may inform therapeutic strategies. These results shown herein may also have implications for vaccine- and T cell-based therapeutic approaches, which seek to exploit neoantigens. Our results suggest it may be important to consider not just whether putative neo-peptides bind the repertoire of HLA alleles, but the expression, copy number and splicing characteristics of each allele. Indeed, the methods described herein may be used to help determine which set of predicted neoantigens are likely to elicit an effective T cell response.

In conclusion, the methods described herein enable accurate estimation of haplotype-specific HLA loss, mutation, as well as expression and splicing from sequencing data, revealing that HLA disruption is a common feature of NSCLC, facilitating immune escape and cancer evolution.

### REFERENCES

Aguiar et al. Methods Mol Biol. 2020; 2120:101-112.
Filip et al. medRxiv Oct. 4, 2020. doi.org/10.1101/2020.09.30.20204875
McGranahan, Nicholas et al. “Allele-Specific HLA Loss and Immune Escape in Lung Cancer Evolution.” Cell vol. 171, 6 (2017): 1259-1271.e11. doi:10.1016/j.cell.2017.10.001
Shukla, Sachet A et al. “Comprehensive analysis of cancer-associated somatic mutations in class I HLA genes.” Nature biotechnology vol. 33, 11 (2015): 1152-8.
Orenbuch R, Filip I, et al. arcasHLA: high-resolution HLA typing from RNAseq. Bioinformatics. 2020 Jan. 1; 36(1):33-40.
Dubois V, et al. A new HLA-B44 allele (B*44020102S) with a splicing mutation leading to a complete deletion of exon 5. Tissue Antigens. 2004 February; 63(2):173-80.
Kawaguchi S, et al. HLA-HD: An accurate HLA typing algorithm for next-generation sequencing data. Hum Mutat. 2017 July; 38(7):788-797.
Horton R, et al. Variation analysis and gene annotation of eight MHC haplotypes: the MHC Haplotype Project. Immunogenetics. 2008 January; 60(1):1-18.
Laks E, et al. Clonal Decomposition and DNA Replication States Defined by Scaled Single-Cell Genome Sequencing. Cell. 2019 Nov. 14; 179(5):1207-1221.e22. doi: 10.1016/j.cell.2019.10.026.
Zaccaria & Raphael. Nature Biotechnology volume 39, pages207-214 (2021).
Shim S, et al. Two-way communication between ex vivo tissues on a microfluidic chip: application to tumor-lymph node interaction. Lab Chip. 2019 Mar. 13; 19(6):1013-1026.
Litchfield K, et al. Meta-analysis of tumor- and T cell-intrinsic mechanisms of sensitization to checkpoint inhibition. Cell. 2021 Feb. 4; 184(3):596-614.e14.
Rooney M S, et al. Molecular and genetic properties of tumors associated with local immune cytolytic activity. Cell. 2015 Jan. 15; 160(1-2):48-61.
McGranahan N, et al. Clonal neoantigens elicit T cell immunoreactivity and sensitivity to immune checkpoint blockade. Science. 2016 Mar. 25; 351(6280):1463-9.
Rizvi N A, Hellmann M D, Snyder A, et al. Cancer immunology. Mutational landscape determines sensitivity to PD-1 blockade in non-small cell lung cancer. Science. 2015; 348(6230):124-128.
Snyder A, Makarov V, Merghoub T, et al. Genetic basis for clinical response to CTLA-4 blockade in melanoma [published correction appears in N Engl J Med. 2018 Nov. 29; 379(22):2185]. N Engl J Med. 2014; 371(23):2189-2199.
Rosenthal R, et al. Neoantigen-directed immune escape in lung cancer evolution. Nature. 2019 March; 567(7749):479-485.
Van Loo P, et al. Allele-specific copy number analysis of tumors. Proc Natl Acad Sci USA. 2010 Sep. 28; 107(39):16910-5.
Carter S L, et al. Absolute quantification of somatic DNA alterations in human cancer. Nat Biotechnol. 2012 May; 30(5):413-21.
Adalsteinsson V A, et al. Scalable whole-exome sequencing of cell-free DNA reveals high concordance with metastatic tumors. Nat Commun. 2017 Nov. 6; 8(1):1324.
Langmead B, Trapnell C, Pop M, Salzberg S L. Ultrafast and memory-efficient alignment of short DNA sequences to the human genome. Genome Biol. 2009; 10(3):R25.
Dobin, A. et al. STAR: ultrafast universal RNA-seq aligner. Bioinformatics 2013 January; 29(1):15-21.
Landau D A, et al. Evolution and impact of subclonal mutations in chronic lymphocytic leukemia. Cell. 2013 Feb. 14; 152(4):714-26.
Roth A, et al. PyClone: statistical inference of clonal population structure in cancer. Nat Methods. 2014 April; 11(4):396-8.
Danaher, P., et al. (2018). Pan-cancer adaptive immune resistance as defined by the Tumor Inflammation Signature (TIS): Results from The Cancer Genome Atlas (TCGA). Journal for ImmunoTherapy of Cancer, 6(1), 1-17.
Dolbier C L, et al. Differences in functional immune responses of high vs. low hardy healthy individuals. J Behav Med. 2001 June; 24(3):219-29.
Momburg F, et al. Loss of HLA-A,B,C and de novo expression of HLA-D in colorectal cancer. Int J Cancer. 1986 Feb. 15; 37(2):179-84.
McGranahan N, et al. Allele-Specific HLA Loss and Immune Escape in Lung Cancer Evolution. Cell. 2017 Nov. 30; 171(6):1259-1271.e11.
Schaafsma E, Fugle C M, Wang X, Cheng C. Pan-cancer association of HLA gene expression with cancer prognosis and immunotherapy efficacy. Br J Cancer. 2021 August; 125(3):422-432. doi: 10.1038/s41416-021-01400-2.
Hanahan D, Weinberg R A. Hallmarks of cancer: the next generation. Cell. 2011 Mar. 4; 144(5):646-74.
Davoli, T., Uno, H., Wooten, E. C., & Elledge, S. J. (2017). Tumor aneuploidy correlates with markers of immune evasion and with reduced response to immunotherapy. Science, 355(6322).
Bentham, R., Litchfield, K., Watkins, T. B. K. et al. Using DNA sequencing data to quantify T cell fraction and therapy response. Nature 597, 555-560 (2021).
Shim J H, et al. HLA-corrected tumor mutation burden and homologous recombination deficiency for the prediction of response to PD-(L)1 blockade in advanced non-small-cell lung cancer patients. Ann Oncol. 2020 July; 31(7):902-911.
Marty Pyke R, et al. Evolutionary Pressure against MHC Class II Binding Cancer Mutations. Cell. 2018 Oct. 4; 175(2):416-428.e13.
O'Donnell T J, Rubinsteyn A, Laserson U. MHCflurry 2.0: Improved Pan-Allele Prediction of MHC Class I-Presented Peptides by Incorporating Antigen Processing. Cell Syst. 2020 Jul. 22; 11(1):42-48.e7.
Jamal-Hanjani M, et al. Tracking the Evolution of Non-Small-Cell Lung Cancer. N Engl J Med. 2017 Jun. 1; 376(22):2109-2121.
Brendan A. et al, Two-pass alignment improves novel splice junction quantification, Bioinformatics, Volume 32, Issue 1, 1 Jan. 2016, Pages 43-49 Gerritsen. RNA analysis of HLA alleles: tidings from the messenger. Doctoral Thesis Maastricht University. 10.26481/dis.20160527 kg
Newman, A. M., Steen, C. B., Liu, C. L. et al. Determining cell type abundance and expression from bulk tissues with digital cytometry. Nat Biotechnol 37, 773-782 (2019).
Lefranc, M.-P. IMGT, the international ImMunoGeneTics database. Nucleic Acids Res. 31, 307-310 (2003).
Liu, P. et al. Benchmarking the Human Leukocyte Antigen Typing Performance of Three Assays and Seven Next-Generation Sequencing-Based Algorithms. Front. Immunol. 12, 652258 (2021).
Veeneman, B. A., Shukla, S., Dhanasekaran, S. M., Chinnaiyan, A. M. & Nesvizhskii, A. I. Two-pass alignment improves novel splice junction quantification. Bioinformatics 32, 43-49 (2016).
Li, H. et al. The Sequence Alignment/Map format and SAMtools. Bioinformatics 25, 2078-2079 (2009).
Castel, S. E., Levy-Moonshine, A., Mohammadi, P., Banks, E. & Lappalainen, T. Tools and best practices for data processing in allelic expression analysis. Genome Biol. 16, 195 (2015).
Benjamin, D. et al. Calling Somatic SNVs and Indels with Mutect2. bioRxiv 861054 (2019) doi:10.1101/861054.
McLaren, W. et al. The Ensembl Variant Effect Predictor. Genome Biol. 17, 122 (2016).
Pedersen, B. S. & Quinlan, A. R. Mosdepth: quick coverage calculation for genomes and exomes. Bioinformatics 34, 867-868 (2018).
Frankell, Dietzen, Al Bakir, et al. The natural history of NSCLC and impact of subclonal selection in TRACERx. Nature, January 2023.
Martinez-Ruiz, Black, Puttick, et al. Genomic-transcriptomic evolution in lung cancer and metastasis. Nature. January 2023.
Krangel, M. S. Secretion of HLA-A and -B antigens via an alternative RNA splicing pathway. J. Exp. Med. 163, 1173-1190 (1986).
Tijssen, H. J., Sistermans, E. A. & Joosten, I. A unique second donor splice site in the intron 5 sequence of the HLA-A*11 alleles results in a class I transcript encoding a molecule with an elongated cytoplasmic domain. Tissue Antigens 55, 422-428 (2000).
Dubois, V., Tiercy, J. M., Labonne, M. P., Dormoy, A. & Gebuhrer, L. A new HLA-B44 allele (B*44020102S) with a splicing mutation leading to a complete deletion of exon 5. Tissue Antigens 63, 173-180 (2004).
Reinders, J. et al. Identification of HLA-A*0111N: a synonymous substitution, introducing an alternative splice site in exon 3, silenced the expression of an HLA-A allele. Hum. Immunol. 66, 912-920 (2005).
Watanabe, Y., Magor, K. E. & Parham, P. Exon 5 encoding the transmembrane region of HLA-A contains a transitional region for the induction of nonsense-mediated mRNA decay. J. Immunol. 167, 6901-6911 (2001).
Bailey, M. H. et al. Comprehensive Characterization of Cancer Driver Genes and Mutations. Cell 174, 1034-1035 (2018).
Berger, A. H. et al. High-throughput Phenotyping of Lung Cancer Somatic Mutations. Cancer Cell 32, 884 (2017).
Martincorena, I. et al. Universal Patterns of Selection in Cancer and Somatic Tissues. Cell 173, 1823 (2018).
Dai, Z.-X., Zhang, G.-H., Zhang, X.-H., Zhu, J.-W. & Zheng, Y.-T. A splice variant of HLA-A with a deletion of exon 3 expressed as nonmature cell-surface glycoproteins forms a heterodimeric structure with full-length HLA-A. Hum. Immunol. 75, 234-238 (2014).
Contini, P. et al. Soluble HLA-A,-B,-C and -G molecules induce apoptosis in T and NK CD8+ cells and inhibit cytotoxic T cell activity through CD8 ligation. Eur. J. Immunol. 33, 125-134 (2003).

All references cited herein are incorporated herein by reference in their entirety and for all purposes to the same extent as if each individual publication or patent or patent application was specifically and individually indicated to be incorporated by reference in its entirety. The specific embodiments described herein are offered by way of example, not by way of limitation. Various modifications and variations of the described compositions, methods, and uses of the technology will be apparent to those skilled in the art without departing from the scope and spirit of the technology as described. Any sub-titles herein are included for convenience only, and are not to be construed as limiting the disclosure in any way. The methods of any embodiments described herein may be provided as computer programs or as computer program products or computer readable media carrying a computer program which is arranged, when run on a computer, to perform the method(s) described above. Unless context dictates otherwise, the descriptions and definitions of the features set out above are not limited to any particular aspect or embodiment of the invention and apply equally to all aspects and embodiments which are described.

Throughout the specification and claims, the following terms take the meanings explicitly associated herein, unless the context clearly dictates otherwise. The phrase “in one embodiment” as used herein does not necessarily refer to the same embodiment, though it may. Furthermore, the phrase “in another embodiment” as used herein does not necessarily refer to a different embodiment, although it may. Thus, as described below, various embodiments of the invention may be readily combined, without departing from the scope or spirit of the invention.

It must be noted that, as used in the specification and the appended claims, the singular forms “a,” “an,” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by the use of the antecedent “about,” it will be understood that the particular value forms another embodiment. The term “about” in relation to a numerical value is optional and means for example +/−10%.

“and/or” where used herein is to be taken as specific disclosure of each of the two specified features or components with or without the other. For example “A and/or B” is to be taken as specific disclosure of each of (i) A, (ii) B and (iii) A and B, just as if each is set out individually herein.

Throughout this specification, including the claims which follow, unless the context requires otherwise, the word “comprise” and “include”, and variations such as “comprises”, “comprising”, and “including” will be understood to imply the inclusion of a stated integer or step or group of integers or steps but not the exclusion of any other integer or step or group of integers or steps.

Other aspects and embodiments of the invention provide the aspects and embodiments described above with the term “comprising” replaced by the term “consisting of” or “consisting essentially of”, unless the context dictates otherwise.

The features disclosed in the foregoing description, or in the following claims, or in the accompanying drawings, expressed in their specific forms or in terms of a means for performing the disclosed function, or a method or process for obtaining the disclosed results, as appropriate, may, separately, or in any combination of such features, be utilised for realising the invention in diverse forms thereof.

1. A method for determining whether an HLA gene has deregulated expression in a tumour sample from a subject, the method comprising:
Obtaining RNA sequence data from the sample;
Obtaining a reference sequence that is specific to one or more HLA alleles identified to be present in the subject;
Aligning the RNA sequence data from the sample to the reference sequence and
Determining whether one or more HLA alleles in the reference sequence have deregulated expression based on: (i) one or more metrics derived from the aligned RNA sequence data at one or more mismatch positions between homologous alleles and/or at one or more non-canonical splice junctions in the reference sequence, and (ii) one or more corresponding reference values.

2. The method of claim 1, whether the deregulated expression comprises an altered level of expression of a specific HLA allele compared to a control level defined by the one or more corresponding reference values, optionally wherein the one or more corresponding reference values are expression levels in one or more normal samples.

3. The method of claim 1 or claim 2, wherein the deregulated expression comprises the presence of an alternative splicing event in the HLA allele, optionally wherein the deregulated expression is the presence of an alternative splicing event that is not present in one or more normal samples and/or wherein the alternative splicing event is an exon skipping event, an intron retention event, a partial exon skipping event, a complete exon skipping event, a partial intron retention event or complete intron retention event.

4. The method of claim 3, wherein determining whether one or more HLA alleles in the reference sequence have deregulated expression comprises determining the number of reads in the aligned RNA sequence data that map, optionally uniquely, to a non-canonical splice junction, wherein the one or more corresponding reference values comprise a predetermined threshold and an alternative splicing event is determined to be present in an HLA allele in the sample if the number of reads that map to a non-canonical splice junction is above a predetermined threshold, optionally wherein the threshold is 20 uniquely mapping reads.

5. The method of any preceding claim, wherein the RNA sequence data is next generation sequencing data, short reads sequence data, and/or whole transcriptome sequencing data, and/or wherein the RNA sequence data is bulk RNA sequence data or single cell RNA sequencing data.

6. The method of any preceding claim, wherein the reference sequence is a genomic reference sequence or a transcriptomic reference sequence, and/or wherein obtaining the reference sequence comprises combining reference sequences for a plurality of HLA allele previously identified to be present in the subject, and/or wherein obtaining the reference sequence comprises identifying one or more HLA alleles present in the subject and combining reference sequences for the one or more alleles identified to be present in the subject,
optionally wherein the HLA alleles present in the subject are identified or have been identified using DNA sequence data from the sample or from a matched sample, wherein a matched sample is a sample that has been obtained from the same subject, and/or
wherein the HLA alleles present in the subject are identified to a level of resolution that specifies at least the allele group and the specific HLA protein.

7. The method of any preceding claim, wherein the method further comprises obtaining one or more corresponding reference values by: obtaining RNA sequence data from one or more normal samples, and for each normal sample: aligning the aligning the RNA sequence data from the normal sample to the reference sequence, and obtaining one or more metrics derived from the aligned RNA sequence data from the normal sample at one or more mismatch positions between homologous alleles and/or at one or more non-canonical splice junctions in the reference sequence,
optionally wherein the deregulated expression comprises an altered level of expression of a specific HLA allele, wherein one or more corresponding reference values are metrics derived from the aligned RNA sequence data from the normal sample(s) at one or more mismatch positions between homologous alleles; and/or
wherein the deregulated expression comprises the presence of an alternative splicing event, and one or more corresponding reference values are derived from the aligned RNA sequence data from the normal sample(s) at one or more non-canonical splice junctions in the reference sequence.

8. The method of claim 6, wherein the one or more metrics derived from the aligned sequence data from the tumour comprise read depths at a plurality of mismatch positions between homologous alleles and the one or more corresponding reference values comprise read depths at the plurality of mismatch positions between homologous alleles derived from the aligned sequence data from the one or more normal samples, and determining whether one or more HLA alleles in the reference sequence have deregulated expression comprises comparing the read depths at mismatch positions in the tumour sample and the normal sample, optionally wherein the comparing is performed using a statistical test to assess the difference between two sets of observations, optionally wherein the statistical test is a paired t-test or a Wilcoxon test, and/or
wherein the one or more metrics derived from the aligned sequence data from the tumour comprise the number of reads that include a non-canonical splice junction and the one or more corresponding reference values comprise the number of reads that include the non-canonical splice junction in the aligned sequence data from the one or more normal samples, and determining whether one or more HLA alleles in the reference sequence have deregulated expression comprises comparing the number of reads that include the non-canonical splice junction in the tumour sample and the one or more normal samples or information derived therefrom, optionally wherein the information derived therefrom comprises an indication of whether the respective numbers of reads indicates the presence of an alternative splicing event in the tumour sample and in the normal sample(s), respectively; optionally wherein the number of reads that include the non-canonical splice junction in the normal or tumour sample may be considered to indicate the presence of an alternative splicing event if it is above a predetermined threshold, such as e.g. 5, 10, 15, 20 or 25 reads.

9. The method of any preceding claims, wherein the one or more reference values are derived from one or more normal samples, optionally wherein the one or more normal samples are matched normal samples or normal samples with the same HLA allele as the tumour sample.

10. The method of any preceding claim, wherein the RNA sequence data comprise RNA sequencing reads, optionally wherein the method further comprises obtaining a normalised read depth for one or more HLA alleles, wherein the normalised read depth is normalised for total coverage and/or allele length, or wherein the method further comprises calculating an adjusted read number for one or more HLA alleles, wherein the adjusted read number for an allele takes into account the number of reads that map uniquely to the allele, the number of reads that map uniquely to the homologous allele, and the number of reads that map to both the allele and the homologous allele,
optionally wherein the normalised read depth is obtained by dividing the, optionally adjusted, number of reads that map to an allele by the length of the allele and/or by dividing: (i) the number of reads that map to an allele, optionally normalised to the total coverage in the sample, by (ii) the number of reads in the RNA sequence data for the sample, and/or wherein the adjusted read depth is obtained by adding (i) the number of reads that map uniquely to the allele, and (ii) the number of reads that map to both the allele and the homologous allele multiplied by a correction factor, wherein the correction factor is the ratio of the number of reads that map uniquely to the allele and the number of reads that map uniquely to either the allele or the homologous allele.

11. The method of any preceding claim, wherein the deregulated expression is an altered level of expression of a specific HLA allele compared to a control level, and wherein the method comprises excluding any allele for which the numbers of reads that map uniquely to one of the two homologous alleles is below a predetermined threshold, optionally wherein the predetermined threshold is 30%, 40%, 50% or 60%, and/or
wherein the deregulated expression is an altered level of expression of a specific HLA gene compared to a control level, optionally wherein the method comprises determining a gene level read depth based on the number of reads that map to one or both homologous alleles, optionally wherein the gene level read depth is normalised for total coverage and/or allele length, such as average allele length, of the homologous alleles.

12. The method of any preceding claim, further comprising determining whether there is allelic imbalance between two homologous alleles in the sample by comparing the read depths at mismatch positions between the two alleles, optionally wherein the comparing uses a statistical test, optionally a Wilcoxon test, and/or wherein the read depths are adjusted such that each sequence read in the RNA sequence data that maps to a mismatch position is counted only once.

13. The method of any preceding claim, wherein the sequence data comprises sequencing reads and aligning RNA sequence data to the reference comprises selecting reads that align to a region comprising the HLA locus in a standard reference sequence and/or selecting reads that contain a sequence that matches to a sequence from a set of target reference sequences and/or selecting reads that do not align to any regions of a standard reference sequence, optionally wherein the match is an exact match, and/or wherein the set of target sequences is a set of k-mers created from the subject-specific reference sequence, and/or wherein the set of target reference sequences comprises all sequences of a predetermined length, such as e.g. 30 bases, in the reference sequences of a set of possible alleles in the subject.

14. The method of any preceding claim, wherein the HLA allele is a class I HLA allele, or wherein the HLA allele is a class II HLA allele.

15. The method of any preceding claim, wherein the method comprises excluding any HLA gene for which the numbers of reads that map to more than one HLA gene is above a predetermined threshold, optionally wherein the predetermined threshold is 5%, 10%, 15% or 20%.

16. The method of any preceding claim, wherein obtaining the reference sequence comprises identifying one or more non-canonical splice junctions in the reference sequence by aligning the RNA sequence data from the sample to reference sequences for one or more HLA alleles identified to be present in the subject, and/or
wherein the method further comprises determining the fraction of cancer cells in the sample that comprise an alternative splicing event in an HLA allele by determining the number of reads that include a non-canonical splice junction in the sample, the number of reads that include a corresponding canonical splice junction, obtaining the ratio of said numbers and dividing the ratio by an estimated cancer cell fraction for the sample, optionally wherein the number of reads are reads that uniquely map to a region containing the non-canonical splice junction or to a region containing the corresponding canonical splice junction.

17. The method of any preceding claim, wherein a read that includes a non-canonical splice junction is a read that comprises sequence from two exons that surround a candidate exon and do not comprise sequence from the candidate exon, a read that comprises sequence from a first exon and a subsequent exon wherein the junction between the first and subsequent exon is within the sequence of the first exon, a read that comprises sequence from a first exon and a preceding exon wherein the junction between the first and preceding exon is within the sequence of the first exon, a read that comprises sequence from an exon and at least a part of the subsequent intron, or a read that comprises sequence from an exon and at least a part of the preceding intron.

18. The method of any preceding claim, wherein the HLA gene is a class I gene, and wherein the non-canonical splice junction involves one or more of exons 2, 3, 4, 5 and introns 2, 3, 4, 5, optionally wherein the non-canonical splice junction results in partial or complete exon skipping of exon 5, partial or complete intron retention of intron 5, partial or complete exon skipping of exons 2, 3 and/or 4, partial intron retention of introns 3 and/or 4, optionally wherein the non-canonical splice junction involves exon skipping of exon 3 and/or exon 5,intron retention of intron 5, complete exon skipping of exon 5, complete exon skipping of exon 3, partial intron retention of intron 5, partial skipping of exons 2, 3 and/or 4, and/or partial intron retention of introns 3 and/or 4.

19. The method of any preceding claim, wherein the reference sequence is a genomic reference sequence and wherein aligning the RNA sequence data from the sample to the genomic reference sequence comprises aligning the RNA sequence data or selected reads from the RNA sequence data to the genomic reference sequence provided as a genomic sequence and an indication of the locations of introns and exons in the genomic sequence, optionally wherein the locations of introns and exons in the genomic sequence comprises one or more known (canonical) locations of introns and exons, optionally wherein the method further comprises identifying using the aligned RNA sequence data the location of one or more candidate non-canonical splice junctions,
optionally wherein the method further comprises excluding candidate non-canonical splice junctions that are supported by fewer than a predetermined number of reads (such as e.g. 2, 3 or 5 reads) and/or re-aligning the RNA sequence data or selected reads from the RNA sequence data to the genomic reference provided as a genomic sequence and an updated indication of the locations of introns and exons in the genomic sequence comprising the known locations of introns and exons and the one or more candidate non-canonical splice junctions.

20. The method of any preceding claim, further comprising identifying one or more somatic mutations in the sequence of one or more HLA alleles present in the subject, optionally wherein identifying one or more somatic mutations in the sequence of one or more HLA alleles present in the subject comprises aligning DNA sequence from the sample to the genomic reference, and/or wherein determining whether one or more HLA alleles in the reference sequence have deregulated expression comprises determining the number of reads that comprise the one or more somatic mutations.

21. The method of any preceding claim, further comprising obtaining said sample from said subject, and/or obtaining RNA and/or DNA sequence data from a tumour sample, and optionally a normal sample, that has been previously obtained from the subject.

22. The method of any preceding claim, further comprising providing to a user, optionally through a user interface, one or more of: one or more of the metrics derived from the aligned RNA sequence data at one or more mismatch positions between homologous alleles, or a metric derived therefrom, such as a read depth for one or both alleles of a pair of homologous alleles in the sample and/or one or more control samples, a normalised read depth for one or both alleles of a pair of homologous alleles in the sample and/or one or more control samples, one or more values quantifying the relative expression of a pair of homologous alleles identified in the subject, an indication of whether an HLA allele is determined to be deregulated in the sample, an indication of whether expression of an HLA allele is determined to be repressed in the sample compared to a control, one or more of the metrics derived from the aligned RNA sequence data at one or more non-canonical splice junctions or a metric derived therefrom, one or more metrics derived from aligned RNA sequence data from one or more normal samples at one or more non-canonical splice junctions or a metric derived therefrom, an indication of whether an HLA allele is determined to have an alternative splicing event in the sample, a number of reads that include a non-canonical splice junction in the sample, an indication of whether an HLA allele is determined to have an alternative splicing event in one or more normal samples, a number of reads that include a non-canonical splice junction in one or more normal samples, a statistical metric (e.g. p value) associated with an indication of whether an HLA allele is differentially expressed in a tumour sample compared to a control level of expression, or a value derived therefrom.

23. A method of providing an immunotherapy for a subject, the method comprising:
(i) identifying one or more neoantigens that are present in the subject;
(ii) determining whether the one or more neoantigens are predicted to be presented by an HLA molecule encoded by an HLA allele that is deregulated in the subject using the method of any preceding claim; and
(iii) providing an immunotherapy that targets a neoantigen of the one or more neoantigens that is predicted to be presented by an HLA molecule encoded by an HLA allele that is not deregulated in the subject.

24. A method of identifying a therapy for a subject that has been diagnosed as having cancer, the method comprising:
(i) identifying one or more neoantigens in the subject to obtain a first neoantigen burden for the subject;
(ii) carrying out the method of any of claims 1 to 22 on one or more tumour samples from the subject to determine whether one or more HLA alleles are deregulated in the subject;
(iii) adjusting the first neoantigen burden for the subject to exclude neoantigens predicted to bind to an HLA allele that has been determined at step (ii) to be deregulated in a tumour from the subject; and
(iv) classifying the subject between a plurality of groups associated with a different responses to CPI therapy based on the adjusted neoantigen burden obtained at step (iii).

25. A system comprising:
a processor; and
a computer readable medium comprising instructions that, when executed by the processor, cause the processor to perform the steps of the method of any of claims 1 to 24.
